(19) United States
(12) Patent Application Publication
(10) Pub. No.: US 2026/0027203 A1
(43) Pub. Date: Jan. 29, 2026

(54) COMPOSITIONS AND METHODS FOR TREATMENT OF CANCER

(71) Applicant: Aleta Biotherapeutics, Inc., Natick, MA (US)

(72) Inventors: Roy Lobb, Chestnut Hill, MA (US); Paul Rennert, Holliston, MA (US); Christine Ambrose, Reading, MA (US)

(21) Appl. No.: 18/854,250

(22) PCT Filed: Apr. 6, 2023
(86) PCT No.: PCT/US2023/017795

§ 371 Date: Oct. 4, 2024

Related U.S. Application Data
(60) Provisional application No. 63/328,485, filed on Apr. 7, 2022.

(51) Int. Cl. A61K 39/395; A61K 38/17; A61K 39/00; A61K 40/11; A61K 40/31; A61K 40/42; A61P 35/00; C07K 14/725; C07K 16/18; C07K 16/28

(52) U.S. Cl. CPC A61K 39/39558; A61K 38/1774; A61K 40/11; A61K 40/31; A61K 40/4211; A61P 35/00; C07K 14/7051; C07K 16/18; C07K 16/2803; C07K 16/2887; A61K 2039/505; C07K 2317/569; C07K 2317/734; C07K 2317/92; C07K 2319/32

(57) ABSTRACT

Fusion proteins and their use in treating subjects having cancer are described.

### SEQUENCE LISTING

The present specification makes reference to a Sequence Listing (submitted electronically as a .xml file named “2012106-0143_SL.xml”). The .xml file was generated on May 19, 2023 and is 105 KB in size. The entire contents of the Sequence Listing are herein incorporated by reference.

### BACKGROUND

The last decade has witnessed a transformation in the treatment of relapsed and refractory B malignancy cancers. The use of CD19-targeting cellular therapeutics can provide high clinical response rates in some of these otherwise difficult-to-treat patient populations. Further, some patients obtain durable clinical remissions that appear curative. These results are tempered, however, by the finding that approximately half of all patients who have an initial response to their cell therapy relapse, and the majority relapse quickly, often within six months. As such, additional therapeutics for treatment of cancers are needed.

### SUMMARY

In some embodiments, the present disclosure provides a fusion protein comprising (i) an antibody, or antigen binding fragment thereof, that binds a tumor antigen; and (ii) a target polypeptide. In one aspect the present disclosure provides a fusion protein comprising (i) an antibody, or antigen binding fragment thereof, that binds a tumor antigen; (ii) a target polypeptide; and (iii) a half-life extension polypeptide. In some embodiments, a fusion protein comprises at least a first linker. In some embodiments, a fusion protein comprises at least a first linker and a second linker.

In some embodiments a half-life extension polypeptide is any one of a hyaluronan binding motif, PAS polypeptide, proline/alanine random coil polypeptide, and albumin protein or fragment (e.g., HSA), polypeptides that bind albumin and an antibody or antigen binding fragment thereof. In some embodiments a half-life extension polypeptide is an anti-albumin antibody or antigen binding fragment thereof.

In some embodiments, antibody or antigen binding fragment thereof is an anti-CD20 antibody or antigen binding fragment thereof. In some embodiments, an anti-CD20 antibody or antigen binding fragment thereof comprises an anti-CD20 VHH. In some embodiments, an anti-CD20 VHH comprises an amino acid sequence of any one of SEQ ID NOs:37, 39, 40, 42-44 and 53.

In some embodiments, a target polypeptide is a B cell antigen. In some embodiments, a target polypeptide is any one of CD19, CD20, CD21, CD22, CD23, CD24, CD40, CD72, CD180, ROR1, BCMA, HLA-DR10, CD1, CD5, CD21, CD25, CD27, CD30, CD38, CD78, CD80, CD86, CD138, CD319, surface Ig, PD-1, PD-L1, PD-L2, TGFbR2, CD79a, and CD79b. In some embodiments, a target polypeptide is CD19 or a fragment or mutant thereof.

In some embodiments, a fusion protein comprises an amino acid sequence having at least about 90%, at least about 95%, or about 100% identity to the amino acid sequence of any one of SEQ ID NOs:2, 4, 6, 8, 14, 16, 18, 20, 26, 28, 30, 32, 34, 36, 50, 52 and 55.

In some embodiments, the present disclosure provides a nucleic acid comprising a nucleotide sequence encoding the amino acid sequence of a fusion protein comprising (i) an antibody, or antigen binding fragment thereof, that binds a tumor antigen; and (ii) a target polypeptide. In one aspect the present disclosure provides a fusion protein comprising (i) an antibody, or antigen binding fragment thereof, that binds a tumor antigen; (ii) a target polypeptide; and (iii) a half-life extension polypeptide. In some embodiments, the present disclosure provides a vector comprising a nucleic acid encoding a fusion protein described herein. In some embodiments, the present disclosure provides a host cell comprising a nucleic acid encoding a fusion protein described herein. In some embodiments, the present disclosure provides a method of producing a fusion protein described herein, the method comprising culturing a host cell comprising a nucleic acid encoding a fusion protein described herein. In some embodiments, the present disclosure provides a method of treating a subject having a tumor, comprising administering to the subject an effective amount of a fusion protein described herein.

In some embodiments, the present disclosure provides an antibody, or antigen-binding fragment thereof, comprising a VHH having the amino acid sequence of any one of SEQ ID NOs: 37, 39, 40, 42-44 and 53, or a fragment thereof. In some embodiments, the present disclosure provides a nucleic acid encoding an antibody, or antigen-binding fragment thereof, comprising a VHH having the amino acid sequence of any one of SEQ ID NOs: 37, 39, 40, 42-44 and 53, or a fragment thereof. In some embodiments, the present disclosure provides a vector comprising a nucleic acid encoding an antibody, or antigen-binding fragment thereof, comprising a VHH having the amino acid sequence of any one of SEQ ID NOs: 37, 39, 40, 42-44 and 53, or a fragment thereof. In some embodiments, the present disclosure provides a host cell comprising a nucleic acid encoding an antibody, or antigen-binding fragment thereof, comprising a VHH having the amino acid sequence of any one of SEQ ID NOs: 37, 39, 40, 42-44 and 53, or a fragment thereof. In some embodiments, the present disclosure provides a method of producing an antibody, or antigen-binding fragment thereof comprising culturing a host cell comprising a nucleic acid encoding an antibody, or antigen-binding fragment thereof, comprising a VHH having the amino acid sequence of any one of SEQ ID NOs: 37, 39, 40, 42-44 and 53, or a fragment thereof.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The figures of the drawing are for illustration purposes only, not for limitation.

FIG. 1 shows the isoelectric point distribution of protein preparations CTE1, CTE2, and CTE3. CTE1 is a mixture of sialylated and non-sialylated protein, CTE2 is predominantly non-sialylated, and CTE3 is predominantly sialylated.

FIG. 2 shows the isoelectric point distribution of protein preparation CTE1 after various treatments to remove sialylation (e.g., sialidase).

FIG. 3 shows the binding affinity curves for protein preparations CTE1 (red circles), CTE2 (green squares), and CTE3 (blue circles) to CD19-negative/CD20-positive JeKo-1 cells (JeKo-19KO), as measured by flow cytometry.

FIG. 4 shows the binding affinity curves for protein preparations CTE1 (green triangles), CTE2 (red circles), and CTE3 (blue squares) to either purified biotinylated human albumin (left panel) or a biotinylated human CD20 membrane preparation (right panel) in vitro, as measured by ELISA.

FIG. 5 shows the kill curve for protein preparations CTE1 (red circles), CTE2 (blue squares), and CTE3 (pink triangles) against CD19-negative/CD20-positive JeKo-1 cells (JeKo-19KO) when administered with CAR-19 T cells.

FIG. 6 shows that wild type JeKo-1 cells are predominantly positive for both CD19 and CD20 expression, as measured by flow cytometry.

FIGS. 7A-7C show the expression levels of CD19 and CD20, as measured by flow cytometry, for JeKo-1 B-cell lymphoma cells treated with CAR-19 T cells over 13 days. Panels 7A, 7B, and 7C correspond to experiments where the ratio of CAR-19 T:JeKo-1 cell ratios of 1:1 (A), 0.3:1 (B), or 0.1:1 (C)

FIG. 8A shows the expression levels of CD19 and CD20, as measured by flow cytometry, for CAR-19 T cells incubated with JeKo-1 cells for 13 days at a ratio of 1:1.

FIG. 8B shows corresponding flow cytometry profiles for subsequent addition of either CAR-19 T cells, fusion protein (middle panel), or both (bottom) to the cells.

FIG. 9 shows a time course of bioluminescent imaging of mice injected with JeKo-19KO lymphoma cells, and treated with either CAR-19 T cells plus CTE1, CAR-19 T cells alone, CAR-20 T cells alone, or left untreated.

FIG. 10 shows a time course of bioluminescent imaging of mice injected with JeKo-19KO lymphoma cells, and treated with either CAR-19 T cells plus CTE1, CAR-19 T cells plus CTE2, CAR-19 T cells alone, or left untreated.

FIG. 11 shows a time course of bioluminescent imaging of mice injected with JeKo-19KO lymphoma cells and treated with varying concentrations of CAR-19 T cells plus CTE2, CAR-19 T cells alone, or left untreated.

FIG. 12 shows mean body weight of mice reported in FIG. 11.

FIG. 13 shows mean luminescence of mice reported in FIG. 11. The graph on the right is an expansion of the plots for 0.016, 0.08, 0.4, and 2 mg/kg fusion protein.

FIG. 14 shows the survival probability for mice reported in FIG. 11.

FIG. 15 shows a protein concentration time course of fusion proteins CTE1, CTE2, and CTE3 in NSG mice serum, as used to estimate protein half-life in circulation in vivo.

FIG. 16 shows the binding affinity curves for various fusion protein constructs to CD20-positive/CD19-negative JeKo-1 cells (JeKo-19KO), as measured by ELISA.

FIG. 17 shows the kill curve for various fusion protein constructs against CD19-negative/CD20-positive JeKo-1 cells (JeKo-19KO) when administered with CAR-19 T cells.

FIG. 18 shows binding and killing curves for additional fusion proteins.

FIG. 19A shows the binding affinity curves for CTE3 fusion protein to CD19-negative/CD20-positive JeKo-1 cells (JeKo-19KO), as measured by flow cytometry.

FIG. 19B shows the binding affinity curves for CTE3 fusion protein to wildtype JeKo-1 cells, as measured by flow cytometry. FIG. 19C shows the binding affinity curves for fusion proteins to wildtype Ramos Hodgkin Lymphoma cells, as measured by flow cytometry.

FIG. 20 shows the kill curve for CTE3 fusion protein preparations against CD19-negative/CD20-positive JeKo-1 cells (JeKo-19KO) when administered with CAR-19 T cells.

FIG. 21A shows CTE3 binding to 293T cells transfected with Cynomolgus Monkey CD20. FIG. 21B shows CTE3 binding to 293T cells transfected with human CD20.

FIG. 22A shows impact of human serum on CAR19 cytotoxicity mediated by CTE3. Increasing levels of CTE3, is shown on the x-axis. HS=human serum. FIG. 22B shows impact of human serum albumin (HSA) on CAR19 cytotoxicity mediated by CTE3. Increasing levels of CTE3, is shown on the x-axis.

FIG. 23A shows both CD79b×CD20 bispecfic CTEs bind biotinylated CD20. FIG. 23B shows CTE #650 binds biotinylated CD79b. FIG. 23C shows binding of mono and bispecific anti-CD20 and anti-CD79b proteins to 293T-CD20 cells. FIG. 23D shows binding of mono and bispecific anti-CD20 and anti-CD79b proteins to 293T-CD79b cells. FIG. 23E shows cytotoxic ability of anti-CD19 CAR T cells plus mono and bispecific anti-CD20 and anti-CD79b proteins on JeKo-1 CD19KO cells.

FIG. 24A shows binding of CTE3 to biotinylated CD20 using SPR. FIG. 24B shows binding of CTE3 to biotinylated CD19 using SPR. FIG. 24C shows binding of CTE3 to biotinylated HSA using SPR.

FIG. 25A shows cytotoxicity curves for JeKo-1 CD19 KO cells after one round of CAR19 stimulation. FIG. 25B shows cytotoxicity curves for JeKo-1 CD19 KO cells after 3 rounds of CAR19 stimulation.

### DEFINITIONS

In order for the present invention to be more readily understood, certain terms are first defined below. Additional definitions for the following terms and other terms are set forth throughout the specification.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time.

Adoptive cell therapy: As used herein, “adoptive cell therapy” or “ACT” involves the transfer of immune cells with anti-tumor activity into cancer patients. In some embodiments, ACT is a treatment approach that involves the use of lymphocytes with anti-tumor activity, the in vitro expansion of these cells to large numbers and their infusion into a cancer-bearing host. In some embodiments, ACT is a treatment approach that involves the use of lymphocytes with anti-tumor activity, their infusion into a cancer-bearing host and in vivo expansion.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amelioration: As used herein, “amelioration” refers to prevention, reduction and/or palliation of a state, or improvement of the state of a subject. Amelioration includes, but does not require, complete recovery or complete prevention of a disease, disorder or condition.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to a polypeptide that includes canonical immunoglobulin sequence elements sufficient to confer specific binding to a particular target antigen. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi-specific antibodies (e.g., Zybodies®, etc), bi- or multi-paratopic antibodies, single chain Fvs, polypeptide-Fc fusions, Fabs, camelid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. As is known in the art, intact IgG antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc, as is known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)). In some embodiments, a protein scaffold generated to bind a target antigen can be used as antigen binding fragment. In some embodiments, a protein binding molecule can be generated in silico based on the amino acid sequence and/or the resolved structure of the protein of interest (e.g., Cao et al. Design of protein binding proteins from target structure alone. Nature, 2022; DOI: 10.1038/s41586-022-04654-9).

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody Fragment: As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi-specific antibodies formed from antibody fragments. For example, antibody fragments include isolated fragments, “Fv” fragments (consisting of the variable regions of the heavy and light chains), recombinant single chain polypeptide molecules in which light and heavy chain variable regions are connected by a peptide linker (“scFv proteins”), recombinant single domain antibodies consisting of a variable region of an antibody heavy chain (e.g., VHH), and minimal recognition units consisting of the amino acid residues that mimic a hypervariable region (e.g., a hypervariable region of a heavy chain variable region (VH), a hypervariable region of a light chain variable region (VL), one or more CDR domains within the VH, and/or one or more CDR domains within the VL). In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)₂ fragment, scFv fragment, Fv fragment, dsFv diabody, dAb fragment, Fd′ fragment, Fd fragment, heavy chain variable region, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Antigen: The term “antigen”, as used herein, refers to an agent that elicits an immune response; and/or an agent that binds to a T cell receptor (e.g., when presented by an MHC molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chimeric antigen receptor (CAR) As used herein “chimeric antigen receptor” or “CAR,” refers to a recombinant cell surface receptor that is engineered to be expressed on an immune effector cell and specifically targets a cell and/or binds an antigen.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fusion protein: As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

Nucleic acid: As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments, a nucleic acid has a nucleotide sequence comprising at least one element that encodes, or is the complement of a sequence that encodes, a polypeptide. In some embodiments, a nucleic acid has enzymatic activity.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. In some embodiments, a polypeptide may be longer than 5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Protein: As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., sialylation, acetylation, amidation, methylation, phosphorylation, etc. In some embodiments, proteins may comprise natural amino acids, non-natural amino acids, synthetic amino acids, and combinations thereof.

Subject: By “subject” is meant a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, or condition (e.g., cancer) has been diagnosed with and/or exhibits one or more symptoms of the disease, disorder, or condition.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, “therapeutically effective amount” refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in one or more pro-angiogenic markers, an increase in anti-angiogenic markers, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vector: As used herein, “vector” refers to a nucleic acid molecule capable of transporting another nucleic acid to which it is associated. In some embodiments, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### Detailed Description of Certain Embodiments

### Overview

The promise of cell therapy for cancer became strikingly clear with the introduction of Chimeric Antigen Receptor (CAR) transduced autologous T cells targeting the B cell malignancy antigen CD19. Anti-CD19 CAR-T cells (CAR-19s) induced high response rates and durable remissions in relapsed and refractory acute lymphocytic leukemia (ALL) and non-Hodgkin lymphoma (NHL) patients (Ruella et al., Curr Hematol Malig Rep. 2016; 11:368-84). Long-term follow-up studies have demonstrated the potential for cures, with some patients approaching 10 years cancer-free (Chong et al., New England Journal of Medicine. Massachusetts Medical Society; 2021; 384:673-4; Neelapu et al., Blood. 2021; 138:93; Cappell et al., J Clin Oncol. 2020; 38:3805-15; Schuster et al., Biology of Blood and Marrow Transplantation. 2019; 25:S20-1; Grupp et al., Biology of Blood and Marrow Transplantation. Elsevier; 2019; 25:S126-7).

However, the cost of CAR-19 therapy remains high, and the requisite infrastructure limits the settings in which CARs can be created and administered (Lin et al., J Clin Oncol. 2019; 37:2105-19). Preparative apheresis, consolidation, lymphodepletion and toxicities can limit the use of CAR-T therapy in frail patients. Side effects from CAR-19 treatment can be pronounced, including grade 3+cytokine release syndrome (CRS) and immune effector cell-associated neurotoxicity syndrome (ICANS) that can lead to prolonged hospitalization and additional cost of care (Brudno et al., Blood Rev. 2019; 34:45-55). More than half of responding patients subsequently relapse from therapy, often within a few months of treatment (Maude et al., New England Journal of Medicine. Massachusetts Medical Society; 2018; 378:439-48; Neelapu et al., New England Journal of Medicine. Massachusetts Medical Society; 2017; 377:2531-44). Many patients relapse due to loss or downregulation of the CD19 protein on their leukemia or lymphoma cells (Shah et al., Nat Rev Clin Oncol. 2019; 16:372-85).

Antigen-loss relapse is a phenomenon driven by natural selection, i.e., the CAR-19 T cells can put intense selective pressure on the population of malignant B cells, driving selection for clones with loss or reduction of the target protein, CD19. Antigen loss can occur via mutational events that prevent expression of the CD19 extracellular domain (ECD) or regions within the ECD, and/or via transcriptional downregulation of expression (Plaks et al., Blood. 2021; 138:1081-5; Majzner et al., Cancer Discov. American Association for Cancer Research; 2018; 8:1219-26). Such escape mechanisms are common across therapeutic modalities in cancer treatment (Donk et al., Blood Cancer Discov. American Association for Cancer Research Journals; 2021; 2:302-18; Ruella et al., Comput Struct Biotechnol J. 2016; 14:357-62). Other mechanisms of escape from CAR-19 therapies have been described and include lineage switch to myeloid cell leukemia and trogocytosis, a process by which CD19 is physically stripped from lymphoma cells by the CAR (Perna, Translational Cancer Research [Internet]. AME Publishing Company; 2016 [cited 2022 Jan. 12]; 5 (available from tcr.amegroups.com/article/view/9016); Miao et al., Frontiers in Immunology. 2021; 12:1862).

Fusion proteins described herein provide a solution to one or more of these issues. In some embodiments, fusion proteins described herein can be expressed by cells transduced with lentiviral, retroviral or other gene therapy vectors, and/or can be expressed by mammalian cell culture in vitro and subsequently purified to create biologics for injection. In some embodiments, fusion proteins of the disclosure include three functional domains: a modified CD19 ECD, an anti-CD20 binding domain, and an anti-albumin binding domain. In some such embodiments, fusion proteins of the disclosure bind to CD20 and display the CD19 ECD. As described herein, such fusion proteins can (i) increase CD19 antigen density on target tumor cells, regardless of their level of CD19 expression, (ii) potently trigger CD19-negative tumor cell death in the presence of CAR-19 T cells in vitro, (iii) prevent antigen-loss relapse escape from CAR-19 therapy, (iv) prevent CD19-negative tumor expansion, e.g., at relatively low doses, and/or (v) have a significant impact on survival. As described herein, fusion proteins of the disclosure can be expressed by transfected mammalian cells, can be efficiently purified and demonstrate favorable biophysical properties indicative that such fusion proteins can be suitable for scaleup and production.

### Fusion Proteins

In some embodiments, the present disclosure provides a fusion protein that comprises or consists of (i) a first antigen binding polypeptide that binds a first tumor antigen and (ii) a target polypeptide. In some embodiments, the present disclosure provides a fusion protein that comprises or consists of (i) a first antigen binding polypeptide that binds a first tumor antigen, (ii) a second antigen binding polypeptide that binds a second tumor antigen, and (iii) a target polypeptide. In some embodiments, a fusion protein described herein comprises or consists of an amino acid sequence that is at least about 85%, 90%, 91%, 92%, 93%, 94%, 95%, 96%, 97%, 98%, 99%, or 100% identical to any one of SEQ ID NOs: 2, 4, 6, 8, 10, 12, 14, 16, 18, 20, 22, 24, 26, 28, 30, 32, 34, 36, 50, and 52, but lacking the disclosed half-life extension polypeptide in each of the sequences. In some embodiments, a fusion protein described herein comprises or consists of an amino acid sequence that is at least about 85%, 90%, 91%, 92%, 93%, 94%, 95%, 96%, 97%, 98%, 99%, or 100% identical to any one of SEQ ID NOs: 2, 6, 10, 14, 18, 22, 26, 30, 34, and 50, but lacking the disclosed half-life extension polypeptide and lacking the disclosed C-terminal hexa-histidine tag (SEQ ID NO: 57) in each of the sequences. In some embodiments, a fusion protein described herein comprises or consists of an amino acid sequence that is at least about 85%, 90%, 91%, 92%, 93%, 94%, 95%, 96%, 97%, 98%, 99%, or 100% identical to SEQ ID NO. 55.

In some embodiments, the present disclosure provides a fusion protein that comprises or consists of (i) an antigen binding polypeptide that binds a tumor antigen, (ii) a target polypeptide, and (iii) a half-life extension polypeptide. In some embodiments, the present disclosure provides a fusion protein that comprises or consists of (i) a first antigen binding polypeptide that binds a first tumor antigen, (ii) a second antigen binding polypeptide that binds a second tumor antigen, (iii) a target polypeptide, and (iv) a half-life extension polypeptide. In some embodiments, a fusion protein described herein comprises or consists of an amino acid sequence that is at least about 85%, 90%, 91%, 92%, 93%, 94%, 95%, 96%, 97%, 98%, 99%, or 100% identical to any one of SEQ ID NOs: 2, 4, 6, 8, 10, 12, 14, 16, 18, 20, 22, 24, 26, 28, 30, 32, 34, 36, 50, and 52. As indicated in the listing of sequences provided herein, each of SEQ ID NOs: 2, 6, 10, 14, 18, 22, 26, 30, 34, and 50 includes (amino to carboxyl terminus): a signal sequence; an anti-CD20 VHH; a first linker (GSGGGGSGGGGS) (SEQ ID NO: 58); a target polypeptide; a second linker (SRGGGGSGGGGSGGGGS) (SEQ ID NO: 59); a half-life extension polypeptide; and a hexa-histidine tag (SEQ ID NO: 57). In some embodiments, a fusion protein described herein comprises or consists of an amino acid sequence that is at least about 85%, 90%, 91%, 92%, 93%, 94%, 95%, 96%, 97%, 98%, 99%, or 100% identical to any one of SEQ ID NOs: 2, 6, 10, 14, 18, 22, 26, 30, 34, and 50, and lacking the C-terminal hexa-histidine tag (SEQ ID NO: 57). As indicated in the listing of sequences provided herein, each of SEQ ID NOs: 4, 8, 12, 16, 20, 24, 28, 32, 36, and 52 includes (amino to carboxyl terminus): an anti-CD20 VHH; a first linker (GSGGGGSGGGGS) (SEQ ID NO: 58); a target polypeptide; a second linker (SRGGGGSGGGGSGGGGS) (SEQ ID NO: 59); and a half-life extension polypeptide.

One of skill in the art will be aware of many amino acid sequences suitable for use as linkers, in addition to those sequences described herein. In some embodiments, the first and/or second linker(s) comprises a series of naturally occurring amino acids. In some embodiments, a linker is derived from a naturally occurring multi-domain protein. In some embodiments a linker is flexible. In some embodiments, a flexible linker is designed using computational tools familiar to those skilled in the art, such as JPred (Dorzdetskiy et al., Nucl Acids Res. 2015; 43: W389-94). In some embodiments a linker adopts a preferred conformation. In some embodiments a linker comprises a GS linker, wherein the linker comprises an amino acid sequences of the formula [GₓS_{y}]_{z}, where x, y, and z, are positive integers. In some embodiments a linker comprises a polyglycine linker, wherein the linker comprises the amino acid sequence Gₓ, where x is a positive integer. In some embodiments a linker comprises a glycine and serine rich linker, wherein glycine and serine collectively comprise 50% or more of the linker amino acid sequence. Many embodiments and applications of specific linker sequences in fusion proteins and methods for linker design have been described in Chen et al., Adv Drug Deliv Rev. 2013; 65: 1357-69; and Liu et al., Bioinformatics. 2015; 31: 3700-2 the contents of each of which are incorporated by reference herein.

### Antigen Binding Polypeptide

In some embodiments, a fusion protein of the disclosure includes an antigen binding polypeptide that binds a tumor antigen, e.g., a tumor antigen described herein. In some embodiments, an antigen binding polypeptide binds CD20. In some embodiments, an antigen binding polypeptide is an anti-CD20 antibody or CD20 binding fragment thereof. In some embodiments, an antigen binding polypeptide is or comprises an anti-CD20 scFv, Fv, or other multi-domain binding fragment. In some embodiments, an antigen binding polypeptide is or comprises an anti-CD20 single domain antibody, e.g., an anti-CD20 single domain antibody described herein.

In some embodiments, a fusion protein of the disclosure includes a first antigen binding polypeptide that binds CD20, and a second antigen binding polypeptide that binds a second tumor antigen described herein (e.g., CD79b, HER-2/neu, c-met, EGFR, Ga733\EpCAM, CD21, ROR1, CLL-1/CLEC12A, HLA-DR10, CD1, CD5, CD21, CD25, CD27, CD30, CD38, CD78, CD80, CD86, CD138, CD319, surface Ig, PD-1, PD-L1, PD-L2, TGFbR2, or BCMA). In some embodiments, the first antigen binding polypeptide is an anti-CD20 antibody or CD20 binding fragment thereof. In some embodiments, the first antigen binding polypeptide is or comprises an anti-CD20 scFv, Fv, or other multi-domain binding fragment. In some embodiments, the first antigen binding polypeptide is or comprises an anti-CD20 single domain antibody, e.g., an anti-CD20 single domain antibody described herein.

### Single Domain Antibodies

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art known, or any future single domain antibodies. Single domain antibodies may be derived from any species including, but not limited to mouse, human, camel, llama, goat, rabbit, bovine. According to one aspect of the disclosure, a single domain antibody used herein is a single domain antibody known as heavy chain antibody devoid of light chains. Such single domain antibodies are disclosed in, e.g., WO 94/04678. Variable domains derived from a heavy chain antibody naturally devoid of light chain (e.g., a single domain antibody) is referred to herein as a “VHH” or “nanobody”. Such a VHH can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the disclosure.

The amino acid residues of VHH domains from Camelids are numbered according to the general numbering for VH domains given by Kabat et al., “Sequence of proteins of immunological interest”, US Public Health Services, NIH (Bethesda, MD), Publication No 91-3242 (1991); see also Riechmann et al., J. Immunol. Methods 231:25-38 (1999). According to this numbering, FR1 comprises the amino acid residues at positions 1-30, CDR1 comprises the amino acid residues at positions 31-35, FR2 comprises the amino acids at positions 36-49, CDR2 comprises the amino acid residues at positions 50-65, FR3 comprises the amino acid residues at positions 66-94, CDR3 comprises the amino acid residues at positions 95-102, and FR4 comprises the amino acid residues at positions 103-113.

It should be noted, however (as is well known in the art for VH domains and for VHH domains), that the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of VH domains, which methods can also be applied in an analogous manner to VHH domains, are known in the art. In the present disclosure, claims and figures, the CDRs are defined according to IMGT numbering (Ehrenmann F., Kaas Q. and Lefranc M.-P. Nucleic Acids Res., 38:D301-D307 (2010) unless indicated otherwise.

### Anti-CD20 VHH

In some embodiments, a fusion protein of the disclosure includes a CD20 binding polypeptide that is or includes an anti-CD20 VHH. In some embodiments, an anti-CD20 VHH comprises or consists of the amino acid sequence of any one of SEQ ID NOs:37-44 and 53, or a CD20-binding fragment thereof.

In some embodiments, an anti-CD20 VHH comprises or consists of an amino acid sequence that is at least 85%, 90%, 91%, 92%, 93%, 94%, 95%, 96%, 97%, 98%, 99%, or 100% identical to the amino acid sequence of any one of SEQ ID NOs: 37-44 and 53, or a CD20-binding portion thereof.

In some embodiments, an anti-CD20 VHH comprises or consists of at least one CDR (e.g., CDR1, CDR2, and/or CDR3) depicted in any one of SEQ ID NOs: 37-44 and 53. In some embodiments, an anti-CD20 VHH comprises CDR1, CDR2, and CDR3 depicted in SEQ ID NO:37. In some embodiments, an anti-CD20 VHH comprises CDR1, CDR2, and CDR3 depicted in SEQ ID NO:38. In some embodiments, an anti-CD20 VHH comprises CDR1, CDR2, and CDR3 depicted in SEQ ID NO:39. In some embodiments, an anti-CD20 VHH comprises CDR1, CDR2, and CDR3 depicted in SEQ ID NO:40. In some embodiments, an anti-CD20 VHH comprises CDR1, CDR2, and CDR3 depicted in SEQ ID NO:41. In some embodiments, an anti-CD20 VHH comprises CDR1, CDR2, and CDR3 depicted in SEQ ID NO:42. In some embodiments, an anti-CD20 VHH comprises CDR1, CDR2, and CDR3 depicted in SEQ ID NO:43. In some embodiments, an anti-CD20 VHH comprises CDR1, CDR2, and CDR3 depicted in SEQ ID NO:44. In some embodiments, an anti-CD20 VHH comprises CDR1, CDR2, and CDR3 depicted in SEQ ID NO:53.

In some embodiments, an anti-CD20 VHH comprises or consists of at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID NOs: 37-44 and 53. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:37; a CDR2 that is at least 85%, 90%, 91%, 92%, 93%, 94%, 95%, 96%, 97%, 98%, 99%, or 100% identical to CDR2 depicted in SEQ ID NO:37; and a CDR3 that is at least 85%, 90%, 91%, 92%, 93%, 94%, 95%, 96%, 97%, 98%, 99%, or 100% identical to CDR3 depicted in SEQ ID NO:37. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:38; a CDR2 that is at least 85%, 90%, 91%, 92%, 93%, 94%, 95%, 96%, 97%, 98%, 99%, or 100% identical to CDR2 depicted in SEQ ID NO:38; and a CDR3 that is at least 85%, 90%, 91%, 92%, 93%, 94%, 95%, 96%, 97%, 98%, 99%, or 100% identical to CDR3 depicted in SEQ ID NO:38. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:39; a CDR2 that is at least 85%, 90%, 91%, 92%, 93%, 94%, 95%, 96%, 97%, 98%, 99%, or 100% identical to CDR2 depicted in SEQ ID NO:39; and a CDR3 that is at least 85%, 90%, 91%, 92%, 93%, 94%, 95%, 96%, 97%, 98%, 99%, or 100% identical to CDR3 depicted in SEQ ID NO:39. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:40; a CDR2 that is at least 85%, 90%, 91%, 92%, 93%, 94%, 95%, 96%, 97%, 98%, 99%, or 100% identical to CDR2 depicted in SEQ ID NO:40; and a CDR3 that is at least 85%, 90%, 91%, 92%, 93%, 94%, 95%, 96%, 97%, 98%, 99%, or 100% identical to CDR3 depicted in SEQ ID NO:40. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:41; a CDR2 that is at least 85%, 90%, 91%, 92%, 93%, 94%, 95%, 96%, 97%, 98%, 99%, or 100% identical to CDR2 depicted in SEQ ID NO:41; and a CDR3 that is at least 85%, 90%, 91%, 92%, 93%, 94%, 95%, 96%, 97%, 98%, 99%, or 100% identical to CDR3 depicted in SEQ ID NO:41. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:42; a CDR2 that is at least 85%, 90%, 91%, 92%, 93%, 94%, 95%, 96%, 97%, 98%, 99%, or 100% identical to CDR2 depicted in SEQ ID NO:42; and a CDR3 that is at least 85%, 90%, 91%, 92%, 93%, 94%, 95%, 96%, 97%, 98%, 99%, or 100% identical to CDR3 depicted in SEQ ID NO:42. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:43; a CDR2 that is at least 85%, 90%, 91%, 92%, 93%, 94%, 95%, 96%, 97%, 98%, 99%, or 100% identical to CDR2 depicted in SEQ ID NO:43; and a CDR3 that is at least 85%, 90%, 91%, 92%, 93%, 94%, 95%, 96%, 97%, 98%, 99%, or 100% identical to CDR3 depicted in SEQ ID NO:43. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:44; a CDR2 that is at least 85%, 90%, 91%, 92%, 93%, 94%, 95%, 96%, 97%, 98%, 99%, or 100% identical to CDR2 depicted in SEQ ID NO:44; and a CDR3 that is at least 85%, 90%, 91%, 92%, 93%, 94%, 95%, 96%, 97%, 98%, 99%, or 100% identical to CDR3 depicted in SEQ ID NO:44. In some embodiments, an anti-CD20 VHH comprises a CDR1 that is at least 85%, 90%, 91%, 92%, 93%, 94%, 95%, 96%, 97%, 98%, 99%, or 100% identical to CDR1 depicted in SEQ ID NO:53; a CDR2 that is at least 85%, 90%, 91%, 92%, 93%, 94%, 95%, 96%, 97%, 98%, 99%, or 100% identical to CDR2 depicted in SEQ ID NO:53; and a CDR3 that is at least 85%, 90%, 91%, 92%, 93%, 94%, 95%, 96%, 97%, 98%, 99%, or 100% identical to CDR3 depicted in SEQ ID NO:53.

Antibodies or fragments can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

### Target Polypeptide

In some embodiments, a fusion protein of the present disclosure includes a target polypeptide. In some embodiments, a target polypeptide is an antigen target for a cellular therapeutic, e.g., a CAR-T cell, an antibody, or an antibody drug conjugate as described in, e.g., WO2017/075537, WO2017/075533, WO2018156802, and WO2018156791.

In some embodiments, a target polypeptide comprises or consists of all or a portion of a tumor associated antigen (TAA) or tumor specific antigen (TSA). Non-limiting examples of TSA or TAA antigens include differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other tumor antigens include TSP-180, MAGE-4, MAGE-5, MAGE-6, RAGE, NY-ESO, erbB, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Rα2, FRα, VEGFR2, Lewis Y, FAP, EphA2, CEACAM5, EGFR, CA6, CA9, GPNMB, EGP1, FOLR1, endothelial receptor, STEAP1, SLC44A4, Nectin-4, AGS-16, guanalyl cyclase C, MUC-1, CFC1B, integrin alpha 3 chain (of a3b1, a laminin receptor chain), and TPS.

In some embodiments, a target polypeptide comprises or consists of all or a portion of a tumor antigen selected from CD19, CD20, CD22, CD30, CD72, CD180, CD171 (L1CAM), CD123, CD133, CD138, CD37, CD70, CD79a, CD79b, CD56, CD74, CD166, CD71, CLL-1/CLEC12A, ROR1, Glypican 3 (GPC3), Mesothelin, CD33/IL3Ra, c-Met, PSCA, PSMA, Glycolipid F77, EGFRvIII, GD-2, MY-ESO-1, and MAGE A3.

In some embodiments, a target polypeptide comprises or consists of all or a portion of a B cell specific marker such as CD19, CD20, CD21, CD22, CD23, CD24, CD40, CD72, CD180, ROR1, BCMA, HLA-DR10, CD1, CD5, CD21, CD25, CD27, CD30, CD38, CD78, CD80, CD86, CD138, CD319, surface Ig, PD-1, PD-L1, PD-L2, TGFbR2, CD79a, and CD79b (see, e.g., LeBien et al., Blood 112:1570-1580 (2008).

CD19 is a 95 kDa type I transmembrane glycoprotein that is used as a biomarker of B cell development (Wang et al., Exp. Hematol. Oncol. 1:36 (2012)). CD19 expression in lymphoma and leukemia has made it an effective therapeutic target, especially for chimeric antigen receptor (CAR) T cell therapy (Maude et al., Blood 125:4017-4024 (2015)). Based on CD19's uniquely efficacious performance in CAR-T cell therapy, therapeutic approaches have been described that involve “converting” CD19− tumors into CD19+ tumors using antibody-CD19 fusions or CD19 variants engineered to bind directly to tumor biomarkers (see, e.g., WO2017/075537 and WO2017/075533).

The extracellular region of CD19 was hypothesized to contain two C2-like immunoglobulin domains (see, e.g., Wang et al., Exp. Hematol. Oncol. 1:36 (2012); Tedder et al., Nat. Rev. Rheumatol. 5:572-577 (2009)). This is supported by homology modeling (Söding et al., Nucleic Acids Res. 33:244-248 (2005)). However, a later published structure demonstrated that CD19 does not include C2-like immunoglobulin domains (Teplyakov et al., Proteins 86:495-500 (2018)). The amino acid sequence of wild type human CD19 is provided herein as SEQ ID NO:47.

In some embodiments, a target polypeptide comprises or consists of all or a portion of the amino acid sequence of SEQ ID NO:47. In some embodiments, a target polypeptide comprises or consists of an amino acid sequence having at least about 95%, 96%, 97%, 98%, 99%, or about 100% identity to the amino acid sequence of SEQ ID NO:47. In some embodiments, a target polypeptide comprises or consists of about 200, 210, 220, 230, 240, 250, 260, 270, or 280 contiguous amino acids of SEQ ID NO:47. In some embodiments, a target polypeptide comprises or consists of all or a portion of a CD19 extracellular domain (ECD). For example, in some embodiments, a target polypeptide comprises or consists of a fragment of SEQ ID NO:47, e.g., a fragment that includes about amino acid 20 to about amino acid 278 of SEQ ID NO:47. In some embodiments, a target polypeptide comprises or consists of all or a portion of the amino acid of SEQ ID NO:48. In some embodiments, a target polypeptide comprises or consists of an amino acid sequence having at least about 95%, 96%, 97%, 98%, 99%, or about 100% identity to the amino acid sequence of SEQ ID NO:48. In some embodiments, a target polypeptide comprises or consists of all or a portion of a CD19 variant or fragment thereof. In some embodiments, a target polypeptide comprises or consists of all or a portion of the amino acid of SEQ ID NO:56. In some embodiments, a target polypeptide comprises or consists of an amino acid sequence having at least about 95%, 96%, 97%, 98%, 99%, or about 100% identity to the amino acid sequence of SEQ ID NO:48. In some embodiments, a target polypeptide comprises or consists of all or a portion of a CD19 variant or fragment thereof. In some embodiments, a CD19 variant is or includes a full length CD19 polypeptide (e.g., SEQ ID NO:47), or a portion thereof, that includes one or more amino acid substitutions described herein. In some embodiments, a CD19 variant is or includes a CD19 ECD (e.g., SEQ ID NO:48 or SEQ ID NO:56), or a portion thereof, that includes one or more amino acid substitutions. In some embodiments, a CD19 variant is or includes a CD19 ECD, or a portion thereof, described in WO2019/118918 the entire contents of which is incorporated herein by reference. In some embodiments, a CD19 variant comprises or consists of the amino acid sequence of SEQ ID NO:46. In some embodiments, a CD19 variant comprises or consists of an amino acid sequence having at least about 95%, 96%, 97%, 98%, 99%, or about 100% identity to the amino acid sequence of SEQ ID NO:46.

### Half-Life Extension

In some embodiments, a fusion protein of the disclosure includes an agent that increases the half-life of the fusion protein, e.g., relative to the fusion protein without the agent. In some embodiments, such an agent is a polypeptide, referred to herein as a “half-life extension polypeptide”.

In some embodiments, the half-life extension polypeptide is a transferrin polypeptide or a portion thereof. Transferrin is recycled by binding to a transferrin receptor (see, e.g., Widera et al., Adv. Drug Deliv. Rev. 55:1439-66 (2003)). In some embodiments, the half-life extension polypeptide is albumin (e.g., bovine serum albumin (BSA), human serum albumin (HSA), or mouse serum albumin (MSA)) or a fragment thereof. In some embodiments, the half-life extension polypeptide is a polypeptide that binds a serum protein. In some embodiments, the half-life extension polypeptide is a serum albumin binder (e.g., a BSA, HSA, or MSA binder).

In some embodiments the serum albumin binder is an albumin binding peptide. Peptides that bind to albumin are described in WO200145746, WO2002076489, WO2008068280, WO2009127691, WO2011095545, and US Pat. Pub. Nos. 20040001827, 20080187517, and 20130316952. One of skill in the art is familiar with methods to link proteins and antibodies directly to albumin or domains of albumin as described, for example in Patterson et al., Bioconjugate Chem. 2016, 27, 10, 2271-2275; Bern et al., Sci. Trans. Med., 14 Oct. 2020•Vol 12, Issue 565.

In some embodiments, the half-life extension polypeptide is or comprises a hyaluronan binding domain. Hyaluronan (HA), also known as hyaluronic acid, is a glycosaminoglycan that is found in connective and other tissues and is abundant in synovial fluid, skin, and the vitreous body. HA binds to a large number naturally occurring hyaluronan-binding proteins (HABPs). Some HABPs contain an HA binding domain referred to as a link module through which they bind to HA (Kohda, C. J et al., Cell 86 (1996) 767-775.). Some HAPBs contain a linear 9-11 residue HA-binding motif containing multiple basic amino acids termed a B-X7-B motif (Yang B., et al. Identification of a common hyaluronan binding motif in the hyaluronan binding proteins RHAMM, CD44 and link protein. EMBO J., 13: 286-296, 1994).

In some embodiments, the half-life extension polypeptide is a PAS polypeptide. As used herein, a “PAS polypeptide” is a polypeptide characterized in that the sum of proline, alanine, and serine residues constitutes more than about 80%, or about 85%, or about 90%, or about 95%, or about 96%, or about 97%, or about 98%, or about 99%, or 100% of the total amino acid sequence of the half-life extension polypeptide. In general, a PAS polypeptide is characterized in that it adopts a random coil conformation under physiological conditions as described in US Pat. Pub. No. 20100292130. PAS polypeptides that may be used as half-life extension polypeptides in the fusion proteins of the present disclosure are further described in WO 2008/155134, US Pat. Pub. No. 20100292130, U.S. Pat. No. 8,563,521 and/or U.S. Pat. No. 9,260,494.

In some embodiments a half-life extension polypeptide consists solely of proline and alanine or consists predominantly of proline and alanine but can have up to 1%, 2%, 3%, 5%, or 10% other amino acid residues. Where other amino acids are present, they may all be the same, or multiple different amino acids may be present. Examples of polypeptides that are composed predominantly or entirely of proline and alanine and adopt a random coil conformation under physiological conditions, referred to as proline/alanine random coil polypeptides, are described in US Pat. Pub. No. 20130072420, U.S. Pat. No. 9,221,882, and/or U.S. Pat. No. 10,081,657.

In some embodiments a half-life extension polypeptide is characterized in that the sum of glycine, alanine, serine, threonine, glutamate, and proline residues constitutes more than about 80%, or about 85%, or about 90%, or about 95%, or about 96%, or about 97%, or about 98%, or about 99%, or 100% of the total amino acid sequence of the half-life extension polypeptide, and the half-life extension polypeptide comprises at least 4 of these 6 different amino acids. Such a polypeptide may be referred to as being composed predominantly of amino acids selected from G, A, S, T, E, and P (see, e.g., US Pat. Pub. No. 20030228309; 9,926,351; 9,976,166; and 10,961,287).

In some embodiments, a half-life extension polypeptide is an antibody or fragment thereof. In some embodiments, a half-life extension polypeptide is an anti-albumin antibody or albumin-binding fragment thereof. In some embodiments, a half-life extension polypeptide is or comprises an anti-albumin single domain antibody. In some embodiments, a half-life extension polypeptide is or comprises an anti-albumin VHH. In some embodiments, an anti-albumin VHH comprises or consists of an amino acid sequence disclosed or described in U.S. Publ. No. 20070269422 the entire contents of which are incorporated herein by reference. In some embodiments, an anti-albumin VHH comprises or consists of the amino acid sequence of SEQ ID NO:45, or an albumin-binding portion thereof. In some embodiments, an anti-albumin VHH comprises or consists of an amino acid sequence that is at least 85%, 90%, 91%, 92%, 93%, 94%, 95%, 96%, 97%, 98%, 99%, or 100% identical to SEQ ID NO:45, or an albumin binding portion thereof.

### Tumors

The present disclosure provides technologies useful in the treatment of any tumor. In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is or comprises a solid tumor, including but not limited to breast carcinoma, a squamous cell carcinoma, a colon cancer, a head and neck cancer, ovarian cancer, a lung cancer, mesothelioma, a genitourinary cancer, a rectal cancer, a gastric cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

### Cellular Therapeutics

In some embodiments, a fusion protein described herein can be administered to a subject as a cellular therapeutic. For example, a nucleotide sequence encoding a fusion protein described herein can be introduced into a cell for administration to a subject as a cellular therapeutic. In some embodiments, a cellular therapeutic can be produced from an immune cell, e.g., a cell useful in or capable of use in adoptive cell therapy. In some embodiments, a cellular therapeutic is produced from a cell type selected from a group consisting of TILs, T-cells, CD8+ cells, CD4+ cells, NK-cells, gamma-delta T-cells, regulatory T-cells, iNKT cells, monocytes, macrophages, IPSC-derived cells or peripheral blood mononuclear cells. As used herein “tumor-infiltrating lymphocytes” or TILs refer to white blood cells that have left the bloodstream and migrated into a tumor. Lymphocytes can be divided into three groups including B cells, T cells and natural killer cells. As used herein “T-cells” refers to CD3+ cells, including CD4+ helper cells, CD8+ cytotoxic T-cells, and delta-gamma T cells.

In certain embodiments, a cellular therapeutic is produced by genetically modifying (e.g., transforming) a cell, e.g., an immune cell, with a nucleic acid encoding a fusion protein described herein. In some embodiments, such nucleic acid is included in a recombinant expression vector. The recombinant expression vector can comprise any type of nucleotides, including, but not limited to DNA and RNA, which can be single-stranded or double-stranded, synthesized or obtained in part from natural sources, and which can contain natural, non-natural or altered nucleotides. A recombinant expression vector can comprise naturally-occurring or non-naturally-occurring internucleotide linkages, or both types of linkages.

A recombinant expression vector can be any suitable recombinant expression vector. Suitable vectors include those designed for propagation and expansion or for expression or both, such as plasmids and viruses. For example, a vector can be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors useful in the context of the disclosure include pBI01, pBI101.2, pBI101.3, pBI121 and pBIN19 (Clontech). Examples of animal expression vectors useful in the context of the disclosure include pcDNA, pEUK-Cl, pMAM, and pMAMneo (Clontech).

In some embodiments, a recombinant expression vector is a viral vector. Suitable viral vectors include, without limitation, retroviral vectors, alphaviral, vaccinial, adenoviral, adeno-associated viral, herpes viral, and fowl pox viral vectors, and preferably have a native or engineered capacity to transform an immune cell (e.g., T cell).

In ex-vivo applications such as cell therapy, gamma retroviral vectors, derived from murine leukemia virus (MLV), were developed first and are still used. Lentiviral vectors, based on human immunodeficiency virus (HIV), are widely used. The general strategy in designing lentiviral vectors is based on the deletion and alteration of the native viral sequences to prevent the generation of replication-competent viruses. Thus, the lentivirus components are segregated into three or four different plasmid constructs with the goal of preventing the possibility of complete recombination to a fully replication competent lentivirus (RCL). The viral vector genome contains at a minimum the transgene expression cassette, the long terminal repeats (LTRs), and the packaging signal. In most cases, three additional plasmids provide the factors required for virus production and packaging (e.g., gag, pol, env). The promoter-enhancer region from the 3′ LTR is also deleted, preventing transcription from this region and subsequent viral replication (termed a self-inactivating vector; SIN). The essential steps of ex-vivo cell transformation or transduction involve cell isolation and culture of the desired cell type to allow the selection, expansion, and differentiation either before or after the cell has been transduced with a viral vector. In the case of hematopoietic cells, most of these steps are performed in a closed system using single-use blood collection and processing bags. For CAR T cell therapy, patient blood cells are harvested, and the desired T cell populations are selected and grown to the required levels. They are then transduced with a viral vector carrying the desired gene cassette, followed by CAR T cell expansion to the billion-cell level. Lentiviral vectors have been shown to transduce T cells efficiently and are, therefore, the preferred vector for introducing CAR into patient target cells. Expanded cells are then reintroduced into the patient.

In certain in-vivo applications, nucleic acids encoding fusion proteins described herein or vectors comprising nucleic acids encoding fusion proteins described herein are administered to an individual in need thereof. For example recombinant expression vectors comprising nucleic acids encoding fusion proteins described herein can be provided as described in, for example, Nawaz et al., Blood Cancer Journal volume 11, Article number: 119 (23 Jun. 2021) Carbonaro-Sarracino et al., Molecular Therapy: Methods & Clinical Development Vol. 16 Mar. 2020; Cantore and Naldini Haemophilia, Volume 27, Issue S3 p. 122-125; Gouze-Decaris, et al., Arthritis Res. 2001; 3(Suppl 1): P34; Breuer et al., Scientific Reports volume 10, Article number: 4544 (2020), Naldini et al., SCIENCE•12 Apr. 1996•Vol 272, Issue 5259•pp. 263-267. Viral vector particles can be used to deliver nucleic acids directly in vivo. Examples of such viral vector particles include lentiviral, retroviral, AAV, HVS, vaccinia and many other viral types. Viral vector particles can be modified for optimized delivery, for example, to specific cell types including immune cells. See, for example, Yang et al., PNAS Aug. 1, 2006 103 (31) 11479-11484; Schaffer et al., Annu Rev Biomed Eng. 2008; 10: 169-194; Lee et al., J. of Controlled Release Volume 334, 10 Jun. 2021, Pages 106-113; Jang et al., Molecular Therapy Volume 19, Issue 8, August 2011, Pages 1407-1415.

Recombinant expression vectors can be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

A recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the recombinant expression vectors include, for instance, neomycin/G418 resistance genes, puromycin resistance genes, hygromycin resistance genes, histidinol resistance genes, tetracycline resistance genes, and ampicillin resistance genes.

Vectors useful in the context of the disclosure can be “naked” nucleic acid vectors (i.e., vectors having little or no proteins, sugars, and/or lipids encapsulating them), or vectors complexed with other molecules. Other molecules that can be suitably combined with the vectors include without limitation viral coats, cationic lipids, liposomes, polyamines, gold particles, and targeting moieties such as ligands, receptors, or antibodies that target cellular molecules.

Vector DNA can be introduced into a cell, e.g., an immune cell (e.g., a T cell), via conventional transformation or transfection techniques. As used herein, the terms “transformation” and “transfection” and “transduction” are intended to refer to a variety of art-recognized techniques for introducing foreign nucleic acid (e.g., DNA) into a cell, including calcium phosphate or calcium chloride co-precipitation, DEAE-dextran-mediated transfection, lipofection, gene gun, or electroporation. Cell lines used to produce viral vector particles can themselves be modified to introduce useful characteristics including, without limitation, non-self shielding polypeptides, cell-type trophic polypeptides, anti-immunosuppression polypeptides and half-life extension polypeptides.

### Protein Therapeutics

In some embodiments, fusion proteins described herein can be produced and administered to a subject as protein therapeutics instead of, or in addition to, being produced by a cellular therapeutic described herein. Such polypeptides can be included in a composition, e.g., a pharmaceutical composition, and used as a protein therapeutic. For example, a protein therapeutic that includes a fusion protein described herein can be administered in combination with a cellular therapeutic, e.g., CAR-T cell or ADC, that targets CD19.

A variety of methods of making polypeptides are known in the art and can be used to make a polypeptide to be included in a protein therapeutic. For example, a polypeptide can be recombinantly produced by utilizing a host cell system engineered to express a nucleic acid encoding the polypeptide. Recombinant expression of a gene can include construction of an expression vector containing a polynucleotide that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA, or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, e.g., BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (e.g., HT1080); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a protein described herein has been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a polypeptide can be fused to heterologous polypeptide sequences to facilitate purification. Alternatively or additionally, a polypeptide or fusion protein can be partially or fully prepared by chemical synthesis.

### Viral Delivery

In some embodiments, a nucleic acid encoding a fusion protein described herein can be introduced into a cell and/or administered to a subject as a viral vector. In some embodiments, such a viral vector can be used to introduce a fusion protein into a cancer cell (e.g., a tumor cell). Introduction of such fusion protein can increase susceptibility to a subject's immune system and/or one or more additional therapeutic agents (see, e.g., WO2017/075533).

### Vector Design

A nucleic acid sequence encoding a fusion protein described herein can be introduced into a number of types of vectors. For example, a nucleic acid can be cloned into a plasmid, a phagemid, a phage derivative, an animal virus, and a cosmid. Other vectors can include expression vectors, replication vectors, probe generation vectors, sequencing vectors, and viral vectors. In other examples, the vector can be a foamy viral (FV) vector, a type of retroviral vector made from spumavirus. Viral vector design and technology is well known in the art as described in Sambrook et al., (Molecular Cloning: A Laboratory Manual, 2001), and in other virology and molecular biology manuals.

### Viral Transduction

Viruses are highly efficient at nucleic acid delivery to specific cell types, while often avoiding detection by the infected host immune system. These features make certain viruses attractive candidates as vehicles for introduction of cellular therapy targets into cancer cells, e.g., solid tumor cells. A number of viral based systems have been developed for gene transfer into mammalian cells. Examples of viral vectors include, but are not limited to, retroviruses, adenoviruses, adeno-associated viruses, herpes viruses, lentiviruses, poxviruses, herpes simplex 1 virus, herpes virus, oncoviruses (e.g., murine leukemia viruses), and the like. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Lentiviral and retroviral transduction can be enhanced by the addition of LentiBOOST (Mayflower Bioscience SBPLV10112) or TransDux (System Biosciences LV850 A-1) or polybrene (SantaCruz sc-134220; Millipore TR-1003-G; Sigma 107689), a cationic polymer (also known as hexadimethrine bromide) that is used to increase the efficiency of the lentiviral or retrovirus transduction.

For example, retroviruses provide a platform for gene delivery systems. Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected gene can be inserted into a vector and packaged in retroviral particles using techniques known in the art. The recombinant virus can then be isolated and delivered to cells of the subject in vivo. A number of retroviral systems are known in the art, (see, e.g., U.S. Pat. Nos. 5,994,136, 6,165,782, and 6,428,953).

Retroviruses include the genus of Alpharetrovirus (e.g., avian leukosis virus), the genus of Betaretrovirus; (e.g., mouse mammary tumor virus), the genus of Deltaretrovirus (e.g., bovine leukemia virus and human T-lymphotropic virus), the genus of Epsilonretrovirus (e.g., Walleye dermal sarcoma virus), and the genus of Lentivirus. In some embodiments, a retrovirus is a lentivirus a genus of viruses of the Retroviridae family, e.g., characterized by a long incubation period. Lentiviruses are unique among the retroviruses in being able to infect non-dividing cells; they can deliver a significant amount of genetic information into the DNA of the host cell, so can be used as an efficient gene delivery vector. In some examples, a lentivirus can be, but not limited to, human immunodeficiency viruses (HIV-1 and HIV-2), simian immunodeficiency virus (SIV), feline immunodeficiency virus (FIV), equine infections anemia (EIA), and visna virus. Vectors derived from lentiviruses offer the means to achieve significant levels of gene transfer in vivo.

In some embodiments, a vector is an adenovirus vector. Adenoviruses are a large family of viruses containing double stranded DNA. They replicate the DNA of the host cell, while using a host's cell machinery to synthesize viral RNA DNA and proteins. Adenoviruses are known in the art to affect both replicating and non-replicating cells, to accommodate large transgenes, and to code for proteins without integrating into the host cell genome.

In some embodiments, an AAVP vector is used. An AAVP vector is a hybrid of prokaryotic-eukaryotic vectors, which are chimeras of genetic cis-elements of recombinant adeno-associated virus (AAV) and phage. An AAVP combines selected elements of both phage and AAV vector systems, providing a vector that is simple to produce in bacteria and can exhibit little or no packaging limit, while allowing infection of mammalian cells combined with integration into the host chromosome. Vectors containing many of the appropriate elements are commercially available, and can be further modified by standard methodologies to include the necessary sequences. Among other things, AAVPs do not require helper viruses or trans-acting factors. In addition, the native tropism of AAV for mammalian cells is eliminated since there is not AAV capsid formation. Other methods and details are in U.S. Pat. No. 8,470,528 and Hajitou A. et al., Cell, 125: 358-398.

In some embodiments, a human papilloma (HPV) pseudovirus is used. DNA plasmids can be packaged into papillomavirus L1 and L2 capsid protein to generate pseudovirion that can efficiently deliver DNA. The encapsulation can protect the DNA from nucleases and provides a targeted delivery with a high level of stability. Many of the safety concerns associated with the use of viral vectors can be mitigated with an HPV pseudovirus. Other methods and examples are in Hung, C., et al., Plos One, 7:7(e40983); 2012, U.S. Pat. No. 8,394,411, and Kines, R., et al Int J of Cancer, 2015.

In some embodiments, an oncolytic virus is used. Oncolytic virus therapy can selectively replicate the virus in cancer cells, and can subsequently spread within a tumor, e.g., without affecting normal tissue. Alternatively, an oncolytic virus can preferentially infect and kill cells without causing damage to normal tissues. Oncolytic viruses can also effectively induce immune responses to themselves as well as to the infected tumor cell. Typically, oncolytic viruses fall into two classes: (I) viruses that naturally replicate preferentially in cancer cells and are nonpathogenic in humans. Exemplary class (I) oncolytic viruses include autonomous parvoviruses, myxoma virus (poxvirus), Newcastle disease virus (NDV; paramyxovirus), reovirus, and Seneca valley virus (picornavirus). A second class (II) includes viruses that are genetically manipulated for use as vaccine vectors, including measles virus (paramyxovirus), poliovirus (picornavirus), and vaccinia virus (poxvirus). Additionally, oncolytic viruses may include those genetically engineered with mutations/deletions in genes required for replication in normal but not in cancer cells including adenovirus, herpes simplex virus, and vesicular stomatitis virus. Oncolytic viruses can be used as a viral transduction method due to their low probability of genetic resistance because they can target multiple pathways and replicate in a tumor-selective method. The viral dose within a tumor can increase over time due to in situ viral amplification (as compared to small molecule therapies which decrease with time), and safety features can be built in (i.e., drug and immune sensitivity).

### Formulation and Administration

Certain embodiments of the disclosure include methods of administering to a subject a cellular therapeutic described herein (or a population thereof), a protein therapeutic described herein, a composition comprising a cellular therapeutic, and/or a composition comprising a protein therapeutic, e.g., in an amount effective to treat a subject. In some embodiments, the method effectively treats cancer in the subject.

In some embodiments a cellular therapeutic comprises an autologous cell that is administered into the same subject from which an immune cell was obtained. Alternatively, an immune cell is obtained from a subject and is transformed, e.g., transduced, with an expression construct described herein, to obtain a cellular therapeutic that is allogenically transferred into another subject.

In some embodiments, a cellular therapeutic is autologous to a subject, and the subject can be immunologically naive, immunized, diseased, or in another condition prior to isolation of an immune cell from the subject.

In some embodiments, additional steps can be performed prior to administration to a subject. For instance, a cellular therapeutic can be expanded in vitro after contacting (e.g., transducing or transfecting) an immune cell with an expression construct described herein, but prior to the administration to a subject. In vitro expansion can proceed for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can proceed for 1-7 days, 2-10 days, 3-5 days, or 8-14 days prior to the administration to a subject.

In some embodiments, during in vitro expansion, a cellular therapeutic can be stimulated with an antigen (e.g., a TCR antigen). Antigen specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded cellular therapeutic can be directly administered into a subject or can be frozen for future use, i.e., for subsequent administrations to a subject.

In some embodiments, a cellular therapeutic is treated ex vivo with interleukin-2 (IL-2) prior to infusion into a cancer patient, and the cancer patient is treated with IL-2 after infusion. Furthermore, in some embodiments, a cancer patient can undergo preparative lymphodepletion—the temporary ablation of the immune system—prior to administration of a cellular therapeutic. A combination of IL-2 treatment and preparative lymphodepletion can enhance persistence of a cellular therapeutic.

In some embodiments, a cellular therapeutic is transduced or transfected with a nucleic acid encoding a cytokine, which nucleic acid can be engineered to provide for constitutive, regulatable, or temporally-controlled expression of the cytokine. Suitable cytokines include, for example, cytokines which act to enhance the survival of T lymphocytes during the contraction phase, which can facilitate the formation and survival of memory T lymphocytes.

In certain embodiments, a cellular therapeutic is administered prior to, substantially simultaneously with, or after the administration of another therapeutic agent, such as a cancer therapeutic agent. The cancer therapeutic agent can be, e.g., a chemotherapeutic agent, a biological agent, or radiation treatment. In some embodiments, a subject receiving a cellular therapeutic is not administered a treatment which is sufficient to cause a depletion of immune cells, such as lymphodepleting chemotherapy or radiation therapy.

A cellular therapeutic described herein can be formed as a composition, e.g., a cellular therapeutic and a pharmaceutically acceptable carrier. In certain embodiments, a composition is a pharmaceutical composition comprising at least one cellular therapeutic described herein and a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

A composition can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular embodiment, the composition employed, the method of administration, and the particular site and subject being treated. However, a dose should be sufficient to provide a therapeutic response. A clinician skilled in the art can determine the therapeutically effective amount of a composition to be administered to a human or other subject in order to treat or prevent a particular medical condition. The precise amount of the composition required to be therapeutically effective will depend upon numerous factors, e.g., such as the specific activity of the cellular therapeutic, and the route of administration, the amount of available antigen or antigens on tumor cells (e.g., as a consequence of tumor bulk or extent of tumor burden) and/or on normal cells, in addition to many subject-specific considerations, which are within those of skill in the art. In some embodiments, the appropriate dose for a cellular therapeutic for a particular cancer indication or indications can be defined in a dose-escalation clinical trial.

Any suitable number of cellular therapeutic cells can be administered to a subject. While a single cellular therapeutic cell described herein is capable of expanding and providing a therapeutic benefit, in some embodiments, 10² or more, e.g., 10³ or more, 10⁴ or more, 10⁵ or more, or 10⁸ or more, cellular therapeutic cells are administered. Alternatively, or additionally 10¹² or less, e.g., 10¹¹ or less, 10⁹ or less, 10⁷ or less, or 10⁵ or less, cellular therapeutic cells described herein are administered to a subject. In some embodiments, 10²-10⁵, 10⁴-10⁷, 10³-10⁹, or 10⁵-10¹⁰ cellular therapeutic cells described herein are administered.

A dose of a cellular therapeutic described herein can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed.

A polypeptide described herein can be incorporated into a pharmaceutical composition (e.g., for use as a protein therapeutic). Pharmaceutical compositions comprising a polypeptide can be formulated by methods known to those skilled in the art (see, e.g., Remington's Pharmaceutical Sciences pp. 1447-1676 (Alfonso R. Gennaro, ed., 19th ed. 1995)). A pharmaceutical composition can be administered parenterally in the form of an injectable formulation comprising a sterile solution or suspension in water or another pharmaceutically acceptable liquid. For example, a pharmaceutical composition can be formulated by suitably combining a polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50, and the like.

Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

Protein half-life can be impacted by the degree of sialylation, which is the post-translational, covalent, addition of terminal sialic acid to glycosylated proteins (Hossler et al., Glycobiology. 2009; 19:936-49). Proteins with more sialyation may have longer half-lives in vivo (Flintegaard et al., Endocrinology. 2010; 151:5326-36; Bork et al., J Pharm Sci. 2009; 98:3499-508). In some embodiments, a fusion protein described herein is formulated as a sialylated protein therapeutic.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the subject. A single dose of a pharmaceutical composition containing a polypeptide can be selected from a range of 0.001 to 1000 mg/kg of body weight. On the other hand, a dose can be selected in the range of 0.001 to 100000 mg/kg of body weight, but the present disclosure is not limited to such ranges. Dose and method of administration can vary depending on the weight, age, condition, and the like of the subject, and can be suitably selected as needed by those skilled in the art.

In some embodiments, a pharmaceutical composition containing a fusion protein is administered in combination with a cellular therapeutic, e.g., CAR-T cell or in combination with an ADC, that targets CD19, as described herein. In some embodiments, a pharmaceutical composition comprises a fusion protein described herein and a cellular therapeutic described herein. In some embodiments, a pharmaceutical composition containing a fusion protein is administered simultaneously, concomitantly, or sequentially with a cellular therapeutic described herein. In some embodiments, a pharmaceutical composition containing a fusion protein is administered about 1 day, 2 days, 3 days, 4 days, 5 days, 6 days, or 7 days, prior to administration of a cellular therapeutic described herein. In some embodiments, a pharmaceutical composition containing a fusion protein is administered about 1 week, 2 weeks, 3 weeks, or 4 weeks prior to administration of a cellular therapeutic described herein. In some embodiments, a pharmaceutical composition containing a fusion protein is administered about 1 month, 2 months, 3 months, 4 months, or 5 months prior to administration of a cellular therapeutic described herein. In some embodiments, a pharmaceutical composition containing a fusion protein is administered about 1 day, 2 days, 3 days, 4 days, 5 days, 6 days, or 7 days, subsequent to administration of a cellular therapeutic described herein. In some embodiments, a pharmaceutical composition containing a fusion protein is administered about 1 week, 2 weeks, 3 weeks, or 4 weeks subsequent to administration of a cellular therapeutic described herein. In some embodiments, a pharmaceutical composition containing a fusion protein is administered about 1 month, 2 months, 3 months, 4 months, or 5 months subsequent to administration of a cellular therapeutic described herein.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only and are not to be construed as limiting the scope or content of the disclosure in any way.

### CAR-T Therapy Relapse

Generally, CAR-T therapy comprises administration of a T-cell expressing a chimeric antigen receptor (CAR) that binds a target antigen. In some embodiments, a CAR-T target antigen is a tumor associated antigen (TAA) or tumor specific antigen (TSA) as described herein.

In some embodiments, the present disclosure is based, in part, on the recognition that certain individuals being treated for cancer with CAR-T therapy who relapse (e.g., cease to exhibit one or more beneficial responses to CAR-T therapy, as described herein) can be “rescued” from relapse by administration of a fusion protein described herein. In some embodiments, the present disclosure provides compositions and methods comprising fusion proteins for the treatment of a subject exhibiting cancer relapse during or after CAR-T therapy.

In some embodiments, the present disclosure is based, in part, on the recognition that certain individuals being treated for cancer with CAR-T therapy will have a suboptimal response to therapy, and therefore may relapse, and therefore are treated to prevent relapse. As one non-limiting example, a suboptimal response may be improved by increasing target antigen density on a tumor cell. In some embodiments, increasing target antigen density on a tumor cell may be achieved by binding a fusion protein of the invention to the tumor cell. In some embodiments, the present disclosure provides compositions and methods comprising fusion proteins for the treatment of a subject expected to have, or having, a sub-optimal responses to CAR T cell therapy, for example, patients who have achieved stable disease, partial response, very good partial response or complete response without achieving minimal residual disease-negative status. (see, e.g., www.cibmtr.org/manuals/fim/1/en/topic/multiple-myeloma-response-criteria).

### Subject Identification

In some embodiments, a subject is identified and/or selected for administration of fusion protein as described herein. In some embodiments, a subject can be identified and/or selected for treatment based on diagnosis of refractory or resistant cancer. In some embodiments, a subject can be identified and/or selected for treatment based on the prescription to receive ACT therapy. In some embodiments, a subject can be identified and/or selected for treatment based on evidence of ACT therapy relapse. In some embodiments, a subject can be identified and/or selected for treatment based on one or more measured or observed sign of relapse in cancer (e.g., a non-beneficial response, loss or downregulation of the target antigen of a cell used in ACT or progressive disease). In some embodiments, the fusion protein is administered to the subject. In some embodiments, upon administration of the fusion protein therapy, the subject exhibits a positive clinical response to the ACT therapy, e.g., exhibits an improvement based on one or more clinical and/or objective criteria (e.g., exhibits a decrease in tumor burden, tumor size, and/or tumor stage).

Methods described herein can include preparing and/or providing a report, such as in electronic, web-based, or paper form. The report can include one or more outputs from a method described herein, e.g., tumor burden, tumor size, and/or tumor stage, stability of disease, loss or downregulation of target antigen. In some embodiments, a report is generated, such as in paper or electronic form, which identifies the presence or absence of one or more tumor antigens for a cancer patient, and optionally, a recommended course of cancer therapy. In some embodiments, the report includes an identifier for the cancer patient. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a cancer patient, e.g., identified in the report. For example, the report can include information, or a recommendation, on the administration of a cancer therapy, e.g., the administration of a pre-selected dosage or in a pre-selected treatment regimen, e.g., in combination with one or more alternative cancer therapies, to the patient. The report can be delivered, e.g., to an entity described herein, within 7, 14, 21, 30, or 45 days from performing a method described herein. In some embodiments, the report is a personalized cancer treatment report.

In some embodiments, a report is generated to memorialize each time a cancer subject is tested using a method described herein. The cancer subject can be reevaluated at intervals, such as every month, every two months, every six months or every year, or more or less frequently, to monitor the subject for responsiveness to a cancer therapy and/or for an improvement in one or more cancer symptoms, e.g., described herein. In some embodiments, the report can record at least the treatment history of the cancer subject.

In one embodiment, the method further includes providing a report to another party. The other party can be, for example, the cancer subject, a caregiver, a physician, an oncologist, a hospital, clinic, third-party payor, insurance company or a government office.

### EXEMPLIFICATION

### Example 1. Construction and Expression of CTE1 and CTE2 Fusion Proteins

Genetic fusion protein constructs comprising a secretion signal peptide, an anti-CD20 VHH, a portion of the CD19 ECD, and an anti-albumin llama VHH were cloned into mammalian cell expression vectors and transiently transfected into Expi293 cells or CHO cells. The cell culture was clarified by centrifugation, and fusion proteins were purified from the supernatant. Two fusion proteins were produced. CTE1 (having the amino acid sequence of SEQ ID NO:2) was expressed in Expi293 cells. CTE2 (having the amino acid sequence of SEQ ID NO:2 and lacking the C-terminal hexa-histidine tag (SEQ ID NO: 57) (e.g., SEQ ID NO. 55) was expressed in CHO cells.

CTE1 fusion protein was purified by His-NTA column chromatography and analyzed by reducing and non-reducing PAGE. Superdex 75 purification was used to isolate the main peak and remove any endotoxin, and SEC-HPLC was used to isolate monomeric protein from the minor aggregates present. CTE2 fusion protein was purified by Protein A affinity chromatography, cation-exchange chromatography, and SEC-HPLC purification. The CTE2 fusion protein was purified to homogeneity as determined by SEC-HPLC and PAGE.

### Example 2. Biophysical Characterization of CTE1 and CTE2 Fusion Proteins

Capillary isoelectric focusing (cIEF) analyses were used to evaluate sialylation of the CTE1 and CTE2 fusion proteins. In a cIEF separation, the continuous pH gradient that is formed by applying voltage across a capillary filled with carrier ampholytes separates proteins by their isoelectric point (pI). The pI of the protein is the pH value at which the total charge on the protein is zero. Therefore, cIEF separates protein isoforms relative to their overall charge, which with respect to fusion protein isoforms is in turn impacted by the presence or absence of sialic acid. In brief, proteins can be evaluated as the percent of basic and acidic peaks, where acidic peaks represent sialylated protein.

Three preparations of protein were evaluated for the extent of sialylation: CTE1 and CTE2 as described in Example 1, and CTE3 (SEQ ID NO. 55), a 100% sialylated form purified from a second batch of CTE2 found to have sialylated and unsialylated forms. CTE3 was prepared by separation using an anion exchange column (AEX) and collecting the eluate.

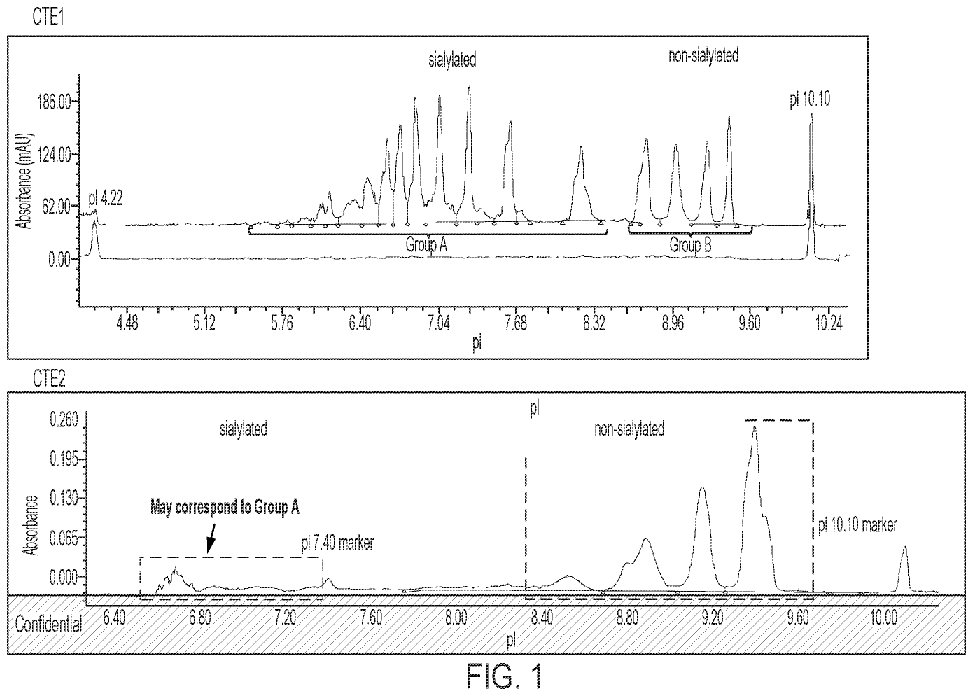

Sialylation was assessed using imaging capillary isoelectric focusing (icIEF), which is a highly resolving technique that separates species primarily on the basis of a molecule's pI intrinsic net charge. icIEF takes into account surface-exposed and internal amino acids, with no loss of resolution due to hydrophobic interactions. icIEF separation is conducted by focusing isoforms in an ampholytic pH gradient through an applied electric field. Following the focusing step is a detection step, which in conventional capillary isoelectric focusing (cIEF) entails chemical or pressure mobilization across a fixed UV detector. icIEF lacks a mobilization step, and detection is achieved through continuously scanning the whole capillary. Because a mobilization step is eliminated, the charge-variant profile can be recorded at its highest possible resolution, within an efficient run time of 15-20 min. The protein preparations CTE1, CTE2 and CTE3 showed distinct icIEF profiles, as shown in FIG. 1. Specifically, CTE1 is a mixture a sialylated and non-sialylated protein, CTE2 is almost wholly non-sialylated protein and CTE3 is almost wholly sialylated protein.

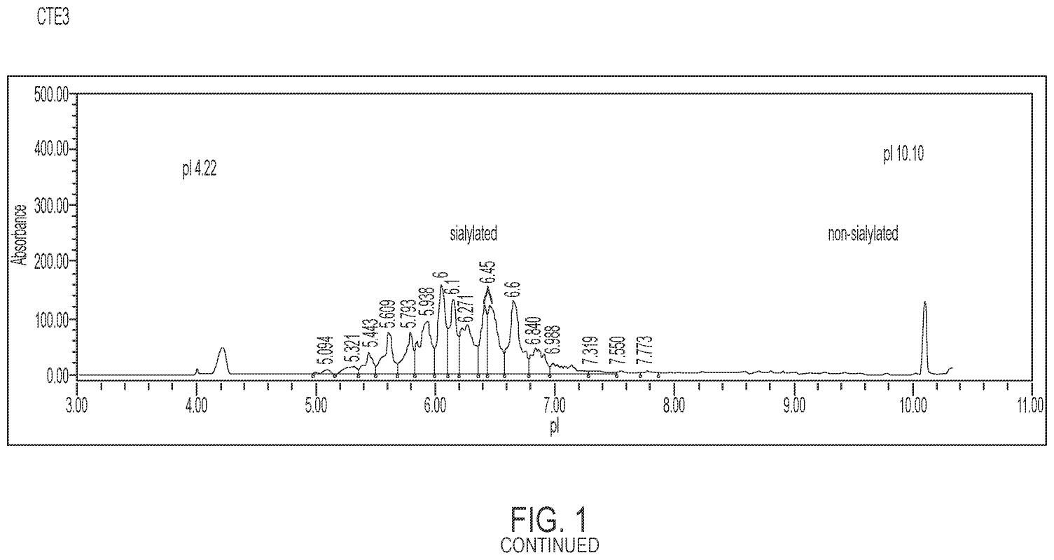

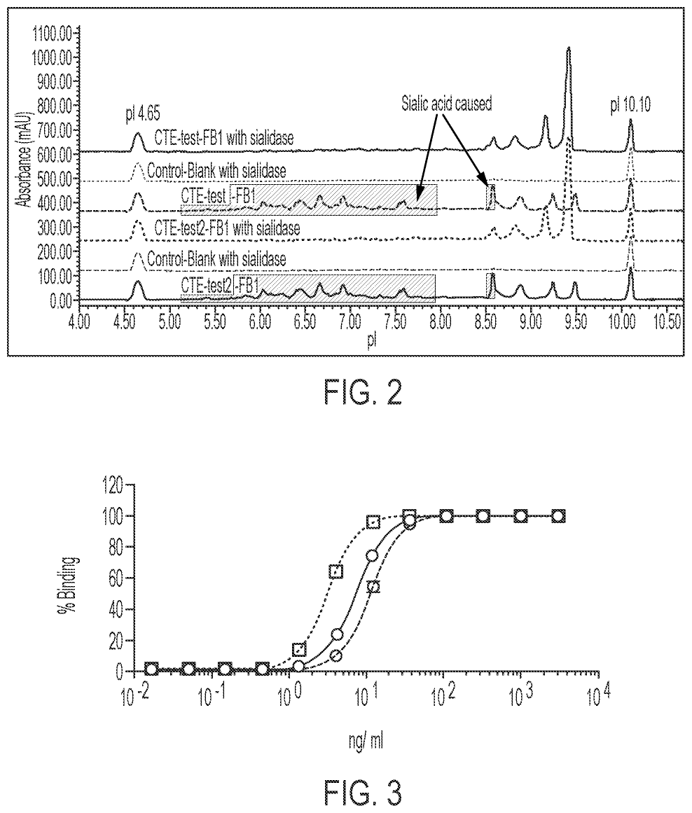

Because the appearance of basic and acidic peaks can be influenced by other features in addition to sialylation, sialidase treatment was performed to show that the presence of acidic peaks was due to the sialic acid modification within the protein preparation. Several CTE1 preparations in different formulation buffers (FB) were evaluated +/−sialidase, these are coded CTE-test and CTE-test2 in FIG. 2.

This experiment shows that sialidase removed all of the more acidic peaks (pH 5.1-pH 7.9) and reduced the size of one basic peak (ph 8.55) slightly.

### Example 3. Fusion Proteins Bind CD20 and Albumin

CTE1 and CTE2 fusion proteins (described in Example 1) and CTE3 (AEX preparation) were assessed for binding to CD19-positive and CD19-negative cells. Briefly, the JeKo-1 mantle cell lymphoma cell line was subjected to CRISPR/Cas9 editing to eliminate CD19 expression, to create the JeKo-19KO cell line. JeKo-19KO cells express CD20 but not CD19. The cells were incubated with the CTE proteins, washed to remove unbound proteins, then incubated with anti-CD19 antibody, FMC63, labeled with phycoerythrin (FMC63-PE). The cells were then washed again and fixated with 2% formalin in PBS for analysis by flow cytometry.

As shown in FIG. 3, CTE1 (red circles), CTE2 (open green squares) and CTE3 (open blue circles) proteins bound equivalently to CD19-negative/CD20-positive JeKo-19KO cell, as detected with anti-CD19-PE antibody (FMC63), that is, without regard to degree of sialylation. The half-maximal effective concentration calculated from these binding data are reported in Table 1.

TABLE 1

| Test molecule | EC₅₀ (ng/ml) | EC₅₀ (nM) |
|---|---|---|
| CTE1 | 7.5 | 0.13 |
| CTE2 | 3.2 | 0.06 |
| CTE3 | 11.5 | 0.2 |

Fusion proteins were assessed for binding to CD20 and albumin. Briefly, two ELISA formats were implemented using the anti-CD19 antibody FMC63 to capture the fusion proteins. ELISA plates (Thermo Fisher) were coated overnight at 4° C. with 1 μg/ml FMC63 and blocked with 200 l/well 0.3% non-fat milk in Tris buffered saline (TBS) for 1 hour at room temperature. The fusion proteins were added in 1% BSA in TBS, in decreasing concentrations, and incubated for 1 hour. The plates were washed 3 times with TBS. Either 0.5 μg/ml biotinylated human albumin (Novus Biologics) or 0.5 μg/ml biotinylated human CD20-“nanodisc” (Acro Biosystems) was then added in 1% BSA in TBS for 1 hour, the plates were washed again, followed by incubation with streptavidin-HRP and TMB peroxidase substrate (Thermo Fisher) to detect bound protein.

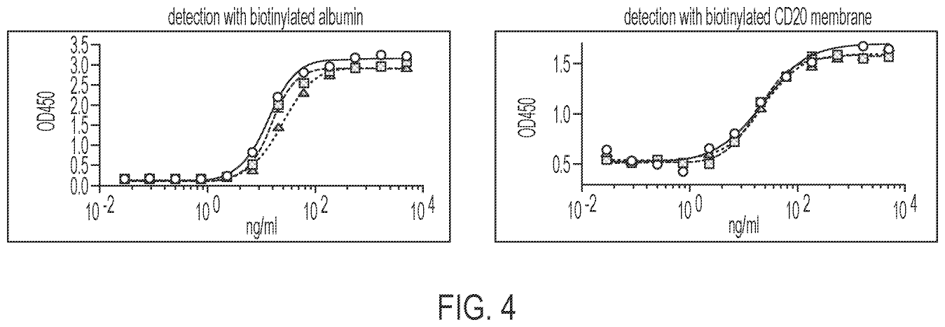

As shown in FIG. 4, CTE1 (green triangles), CTE2 (red circles) and CTE3 (blue squares) proteins bound equivalently to anti-CD19-coated plates, as detected with biotinylated albumin (left) or biotinylated CD20 membrane preparation (right), that is, without regard to degree of sialylation. The half-maximal effective concentration calculated from these binding data are reported in Table 2.

TABLE 2

| | EC₅₀ ng/ml | EC₅₀ ng/ml |
|---|---|---|
| Test molecule | (albumin-bio) | (CD20-bio) |
| CTE2 | 13.5 | 22.3 |
| CTE1 | 24.6 | 22.0 |
| CTE3 | 15.1 | 18.6 |

### Example 4. Fusion Proteins Mediate CAR-19 T Cell Targeting and Cytotoxicity

CTE1 and CTE2 fusion proteins (described in Example 1), were assessed for CAR-19 T cell targeting and cytotoxicity. Briefly, 96-well round bottom plates were seeded with 50 μL of JeKo-1 CD19KO target cells carrying the luciferase gene at 1×10⁴ cells/well in RPMI 1640 medium containing 10% FBS without antibiotics (RPMI/FBS). Dilutions of test proteins were made in 50 μL RPMI/FBS and added to the cells. The CAR-19 T cells were thawed and washed once with RPMI/FBS, collected via centrifugation at 550 RCF for 10 minutes and added to the wells in a volume of 50 μL to give defined CAR-19 T:target (E:T) cell ratios. The plates were incubated at 37° C. for 48 hours. The plates were centrifuged at 550 RCF for 5 minutes, the pellet was rinsed with PBS, and spun again. Then, 20 μL of 1×lysis buffer (Promega) was added to the pellet, and the lysate was transferred into 96 well opaque tissue culture plates (Fisher Scientific). The plates were read in a luminometer with an injector to dispense the substrate (Promega). The percent killing was calculated based upon the average loss of luminescence of the experimental condition vs the control condition of target cell plus CAR-19 T cell only.

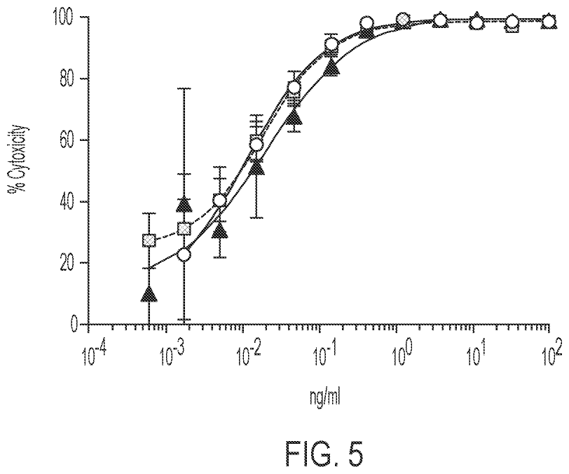

As shown in FIG. 5, CAR-19 T cells had equivalent cytotoxic activity in the presence of target JeKo-19KO cells and CTE1 (red circles), CTE2 (blue squares) and CTE3 (open triangles) proteins, that is, without regard to degree of sialylation. The half-maximal inhibitory concentration calculated from these cytotoxicity data are reported in Table 3.

TABLE 3

| | CTE1 | CTE2 | CTE3 |
|---|---|---|---|
| Cytotoxicity | 0.012 | 0.018 | 0.020 |
| IC50 (ng/ml) | | | |

These results show that CTE fusion proteins can redirect CAR-19 T cells to CD19 that is bound to and displayed on CD20 cell surface protein. Fusion proteins that bind to CD20 and display the extracellular domain of CD19 can increase the apparent density of CD19 on wildtype lymphoma cells that naturally express both antigens. Using the “Bang Beads” methodology (Bangs Laboratories, Fishers, IN USA), the number of apparent CD19 molecules on the surface of a lymphoma cell is shown to increase in a dose-dependent manner when CTE3 is added to either wildtype JeKo-1 Mantle Cell Lymphoma cells or wildtype Ramos Hodgkin Lymphoma cells. The total number measured is found to approximately equal to the number of CD19 receptors plus the number of CD20 receptors as measured by flow cytometry staining. The experiments are performed using the manufacturer's directions.

### Methods

Beads staining (Bangs Lab QSC beads cat #: 815B5ML; anti-mouse IgG, lot 15515, access code: 22020217-1): Manually shake the bottle to ensure uniform suspension. Do not vortex. The blank population: Do not stain. Stain each labeled population (1-4) separately. Add one drop of QSC beads to 50 μL FACS buffer, gently tap to mix. Add 30 μL of FMC63-PE (amount for 3×10⁶ cells) for CD19 standard curve beads, add 60 μL of a-CD20-PE (amount for 3×10⁶ cells), gently tap to mix immediately. Incubate in dark for 30 minutes at 4° C. Add 1 ml of FACS buffer and spin at 2500 g for 5 minutes. Wash 2 times with 1 ml of FACS buffer and spin at 2500 g for 5 minutes; resuspend in 500 μL PBS.

Cell staining: In 96 well FACS plate, incubate 50 μL Fc blocked cells (˜5×10⁵ with 50 μL 3×serial diluted 606 protein (CTE3, SEQ ID NO. 55, Sialylated) for 30 minutes at 4° C. Wash the cells 2 times with 200 μL FACS buffer. Resuspend the cells in 50 μL of FACS buffer, add 30 μL of FMC63-PE and incubate for 30 minutes at 4° C. For controls: a. unstained cells, b. FMC63-PE stained cells (30 μL/test for 3×10⁶ cells), c. anti-CD20-PE stained cells (60 μL of anti-CD20-PE for 3×10⁶ cells), gently tap to mix immediately. Incubate the cells in the dark for 30 minutes at 4° C. Wash the cells 3 times with 200 μL of FACS buffer and spin at 500 g for 2 minutes. Resuspend the cells in 200 μL PBS with 1% paraformaldehyde to fix the cells for 30 minutes. FACS analysis: run the beads and cells on the same day at the same settings.

Analyzing the beads on Accuri C6 plus machine: set the flow rate at 100-200 events/second (Fluidics at medium), collect 1000 events per bead population. The stained beads may be combined and run in a single tube (5×1000 events). Using FSC/SSC dot plot, create FSC-H/FSC-A to get singlet and SSC-A/FL2 (PE channel). Create a Histogram/FL2 (PE channel) and apply a half-height/full width gate to each bead population (M1, M2, M3, M4, M5). Record the Geo Mean or Median of FL2 (PE channel) for each bead population for entry into QuickCal spreadsheet (down load QuickCal v 3.0 using an access code). Enter the Median of FL2 (PE channel) to get the standard curve.

Run the cells on the Accuri C6 plus at the same setting as beads analysis, collect 5000 events/test. Using FSC/SSC dot plot, create FSC-H/FSC-A to get singlet and SSC-A/FL2 (PE channel). Create a histogram/FL2 (PE channel) and apply a half-height/full width gate to the cell population. Record the Geo Mean or Median of FL2 (PE channel) for the cell population for entry into QuickCal spreadsheet. Enter the Median of FL2 (PE channel) and get the ABC (Antibody Binding Capacity) values. Subtract the ABC value of either unstained cell population or isotype ctrl from the cell's ABC value. If monovalent antibody-to-surface receptor binding is presumed, then the ABC values=#surface receptors. The results show that the apparent number of CD19 molecules on the surface of a JeKo-1 cell visibly increases in a dose dependent manner in a range between 10 ng/ml and g/ml CTE3. The results show that the apparent number of CD19 molecules on the surface of a Ramos cell visibly increases in a dose dependent manner in a range between 10 ng/ml and 10 μg/ml CTE3. The results show that the effect of increasing the number of CD19 molecules on a target lymphoma cell enhances CAR-19 mediated toxicity for up to 24 hours, at diverse E:T ratios. The results show enhanced cytotoxicity within a range of E:T ratios that include 0.1:1, 0.3:1, 1:1, 3:1 and 5:1.

The conclusions are that CTE3 increases the apparent CD19 density on lymphoma cells to a degree that is sufficient to increase CAR-19 mediated cytotoxicity.

### Example 5. Treatment of CD19 Antigen Escape with Fusion Proteins

CTE1 fusion protein (described in Example 1) was assessed for the ability to modulate target cell CD19 antigen escape. To generate a CD19 antigen escape model, wild type CD19-positive/CD20-positive JeKo-1 cells carrying the luciferase gene (JeKo-luc) were seeded at 1×10⁴ cells in 100 μl per well of RPMI/FBS in 96 well round bottom plates. CAR-19 T cells in 100 μl RPMI/FBS were added to give JeKo-luc:CAR-19 T cell ratios of 1:1, 0.3:1, or 0.1:1 and the co-culture was incubated for up to 13 days. Samples were evaluated by flow cytometry at days 1, 6, 11 and 13 for the accumulation of a CD19− CD20+ population (FMC63-PE, Millipore; anti-CD20-APC, BD Pharmingen).

Once a CD19-negative population emerged, the JeKo-luc cells were replated at 1×10⁴ cells in 50 μl RPMI/FBS, after counting by flow cytometry using bead counting, following the manufacture's protocol (Bang Laboratories, Inc). Then, 1×10⁵ CAR-19 T cells in 50 μl RPMI/FBS were added to give a JeKo-luc:CAR-19 T ratio of 10:1, with or without fusion protein in 50 μl RPMI/FBS. In a set of control wells the fusion protein was added to the JeKo-luc cells alone, without CAR-19 T cells. In each case, the 150 μl co-culture was incubated for 48 hours. Then, the cells were stained and analyzed by flow cytometry with HIB-CD19-PE (BioLegend) and anti-CD20-APC (BD Pharmingen) for JeKo-1 cells not incubated with protein or with anti-CD20-APC and anti-ROR1-PE (BioLegend) antibodies for samples treated with protein. 7AAD was used to gate out dead cells.

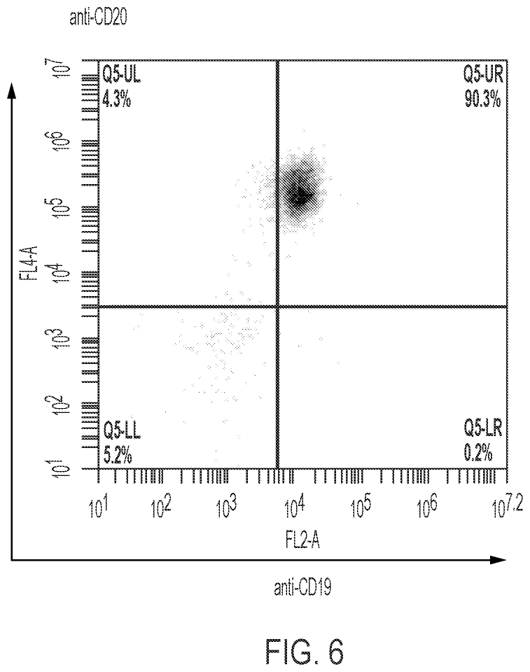

Wild type JeKo-1 cells have a very high proliferative index, and they were used as a model of antigen escape from CAR-T cell therapy. The phenotype of these cells under CAR-T pressure was first characterized. JeKo-1 cells are CD19-positive and CD20-positive and the vast majority are double positive as shown in FIG. 6.

FIG. 6 shows that wild type JeKo-1 cells express CD19 (X-axis) and CD20 (Y-axis), therefore nearly all cells (90%) are in the upper right quadrant, indicating dual positivity.

Using a titration of effector:target ratio (E:T, here referring to CAR-T:JeKo-1) whereby the number of CAR-T cells was varied versus a fixed number of JeKo-1 cells, and further, by varying the length of time in culture, a clear antigen escape model was created.

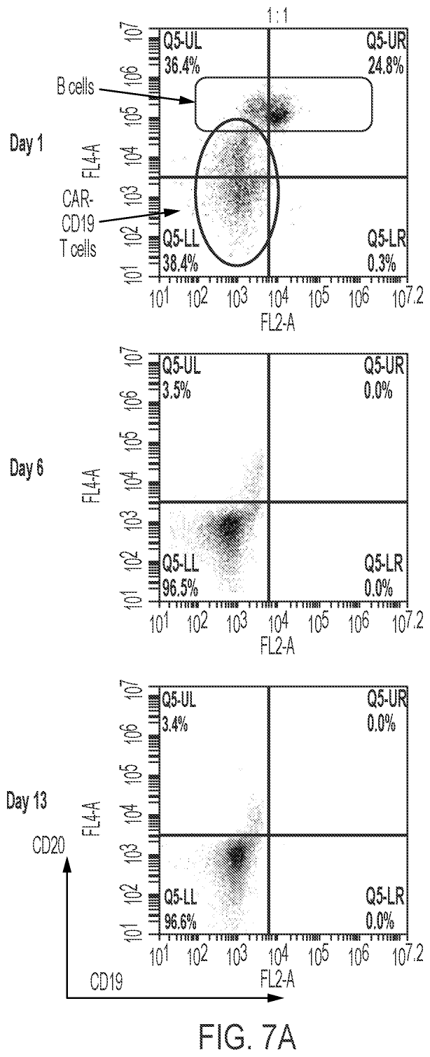

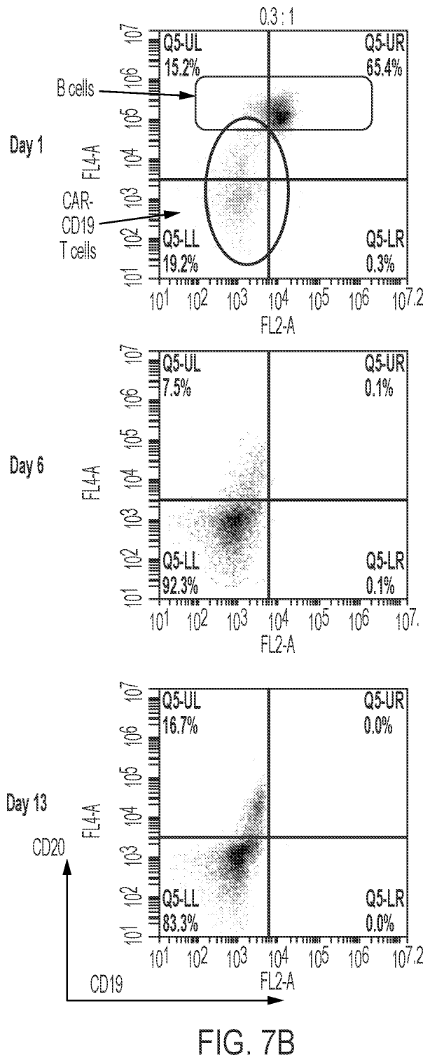

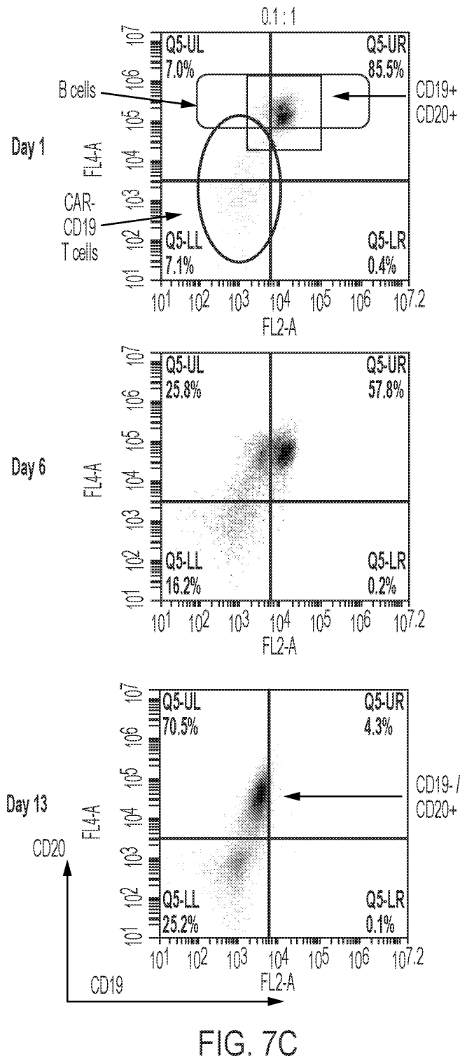

FIG. 7 shows the development of a JeKo-1 antigen escape model from treatment with CAR-19 T cells in vitro. The position of CAR-19 cells and the target JeKo-1 cells is indicated in the flow cytometry profiles is shown to the left of the panels. The designation of CD19-positive and CD20-positive cell populations is shown to the right of the panels. A) E:T ratio 1:1 from day 1-13. B) E:T ratio 0.3:1 from day 1-13. C) E:T ratio 0.1:1 from day 1-13.

As can be seen in FIG. 7, JeKo-1 cells can escape from CAR-19 cytotoxicity via cell population-level loss of CD19 expression. This occurs even at a 1:1 E:T ratio as seen in FIG. 7A, days 6 and 13. The effect is more pronounced at the 0.3:1 and 0.1:1 E:T ratios as seen in FIG. 7B (days 6 and 13) and FIG. 7C, where the effect is especially pronounced at day 13. Notably, the 0.1:1 E:T ratio was ineffective at the level of cytotoxicity readout in the luciferase assay, yet the selective pressure imposed by the CAR induced a shift from a mainly double-positive (CD19 and CD20 positive) population (FIG. 7C, day 1) to a mixed population (FIG. 7C, day 6) to a population dominated by CD19-negative, CD20-positive cells (FIG. 7C, day 13).

Since escapes occurred across this range of E:T ratios and days of incubation, a second study was set up to evaluate the effect of adding the CD19-anti-CD20 fusion proteins.

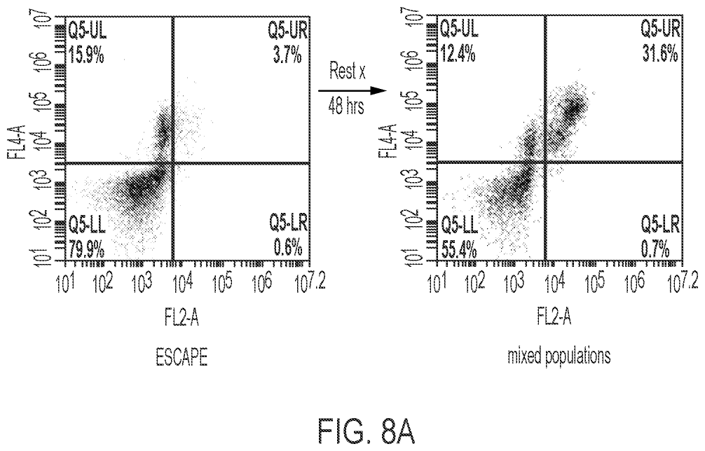

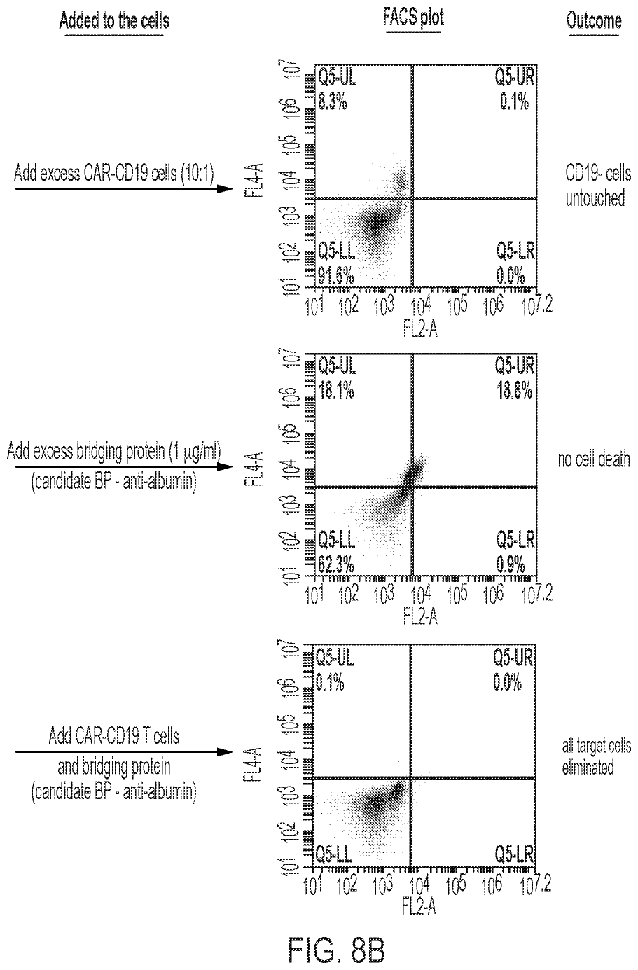

FIG. 8A shows the CD19 (x-axis) and CD20 (y-axis) expression levels of CAR-19 cells incubated with JeKo-1 cells (E:T 1:1) for 13 days, as measured by flow cytometry. Expression of CD19 and CD20 was monitored by FACS. FIG. 8B shows the outcome when only CTE1 protein, only CAR-19 T cells, or both CTE1 and CAR-19 T cells were added to the culture.

In this experiment the addition of CAR-19 T cells and CTE1, but not the CAR-19 T cells alone nor the CTE1 protein alone, was sufficient to kill all target JeKo-1 cells post escape.

These results show that antigen escape, specifically from CD19 expression, can be induced in vitro and reversed using the CD20-targeting CTE1.

### Example 6. In Vivo Treatment of Cancer in Mice with Fusion Proteins

CTE1 and CTE2 fusion proteins (described in Example 1) were assessed for tumor treatment in mouse models. Briefly, female NOD-scid IL2Rgamma^{null} (NSG) mice, 6-10 weeks old were obtained (Jackson Labs) and allowed to acclimate in the vivarium for a minimum of 3 days prior to study initiation. All animals were socially housed in static, sterile bio-contained disposable cages pre-filled with corn cob bedding. Food (LabDiet) and acidified water were provided ad libitum. Mice were injected IV with 0.1 mL/animal of 2.5×10⁶ JeKo-19KO cells on study day 1. On study day 3, all animals were imaged and then randomized to give equivalent average tumor burden in each of 6 different cohorts of n=8 mice/cohort. The following day, study day 4, different cohorts of mice were given doses ranging from 0 to 5 mg/kg of fusion protein, depending on the study, followed by 1×10⁷ CAR-19 T cells 3-4 hours later. Fusion protein was then dosed three times weekly for up to a total of 14 injections. Control cohorts were mice treated with CAR-19 T cells only, with no protein injected, and untreated mice that received no therapeutics.

At least once daily, animals received a cage side health check and clinical observation were performed. Clinical observations were performed more often if abnormal clinical signs were exhibited by animals on study. Body weights were recorded prior to tumor induction, CAR-19 T cell administration, and at three times weekly thereafter unless otherwise was indicated by animal health. The most recently collected body weights were used to calculate protein and D-luciferin doses. Imaging was performed on day 3 in order to randomize animals into cohorts having similar average baseline luminescence. Following infusion of CAR-19 cells and CTE proteins, whole body imaging was performed twice weekly for all animals 10-15 minutes after subcutaneous dosing with 15 mg/mL luciferin in 0.2 mL PBS. Isoflurane was administered during the procedure. Values higher than 1×10¹⁰ total flux, as measured in lumins, were at maximum and were considered cause for humane euthanasia.

Several studies using the JeKo-19KO cell line, CTE proteins and CAR-19 T cells were performed. Animals were injected with JeKo-19KO lymphoma cells which were allowed to implant for 4 days prior to treatment initiation with CAR-19 T cells and CTE1 protein. CAR-19 T cells were injected once, and CTE1 protein was given 3× weekly. Tumor burden was quantified using luciferase-mediated luminescence.

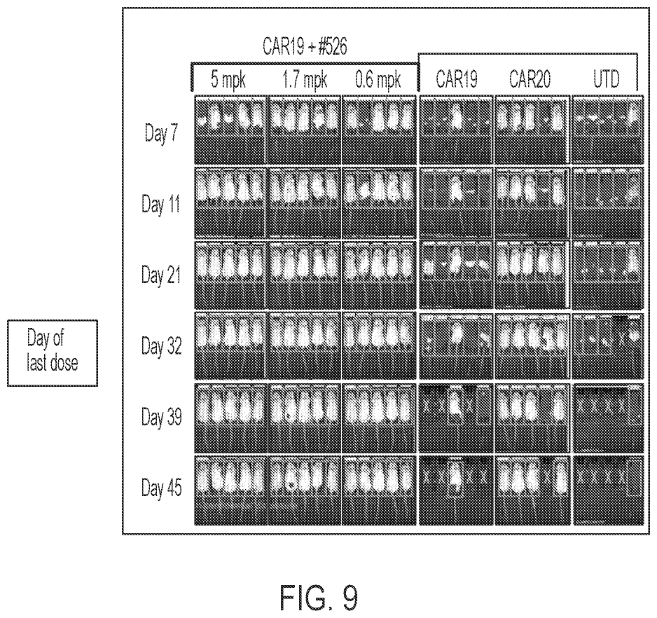

In the first study the CTE1 protein was tested for in vivo efficacy. A dose of 2.5×10⁶ JeKo-CD19-KO cells tumor cell dose was injected intravenously into NSG mice. CAR-19 T cells (1×10⁷) and CTE1 were added on Day 3. CTE1 was then dosed 3×/weekly but was stopped on day 32 to monitor for lymphoma recurrence. As shown in FIG. 9, mice treated with anti-CD19 CAR T cells and the CTE1 were cured, and through day 45 no recurrence was noted. CAR-19 without CTE1 and CAR-CD20 cohorts were included; as expected the CAR-19 treated animals developed lymphoma and 4 of 5 expired by day 45, as was also true of untransduced T cell control cohort (UTD). The CD20 CARs worked well except for one animal that developed lethal systemic lymphoma.

These results demonstrate that mice treated with CAR-19 T cells plus CTE1 protein were protected from the lethal lymphoma. Additionally, durable responses were achieved after dosing cessation, even at the lowest dose used, 0.56 mg/kg.

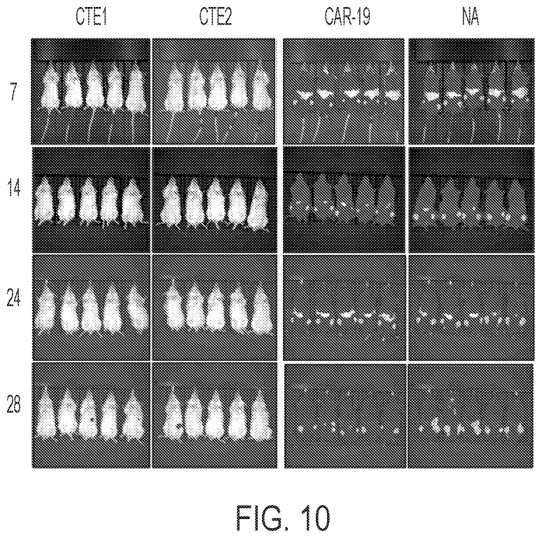

In the second study the CTE1 and CTE2 protein preparations were screened for activity at a dose of 2 mg/kg protein as compared to a CAR-19 T cells only cohort and an untreated cohort. As shown in FIG. 10, thrice weekly dosing with CTE1 and CTE2 proteins restrained lymphoma development through day 31, at which time the untreated (NA) mice and the CAR-19 treated mice began succumbing to disease.

These results demonstrate that at 2 mg/kg dose, the CTE1 and CTE2 proteins had a similar impact on lymphoma growth in vivo.

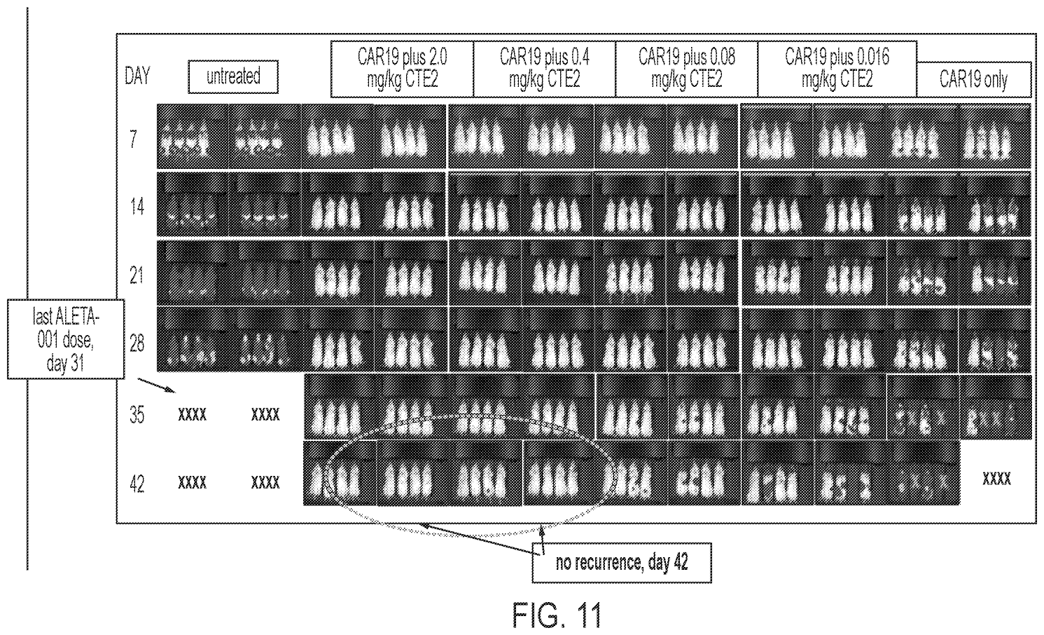

In a third study, two parameters were further investigated. First, a dose titration of CTE2 was performed across 4 cohorts: 2 mg/kg, 0.4 mg/kg, 0.08 mg/kg and 0.016 mg/kg. Second, animals were dosed with protein until day 31 at which time dosing was stopped in order to evaluate animals for signs of lymphoma relapse. Nine days later, on day 42, there was no sign of luminescence in animals treated with CAR-19 T cells plus 2 mg/kg CTE2 and only a minimal signal was seen in 2 of 8 animals treated with CAR-19 T cells plus 0.4 mg/kg CTE2, as shown in FIG. 11. Lower doses of CTE2 were less effective in preventing relapse after dosing cessation.

These results demonstrate that dosing with CAR-19 T cells and CTE2 prevented recurrence of lymphoma in all of the mice in the highest dose cohort of 2 mg/kg and in the majority of mice in the 0.4 and 0.08 mg./kg cohorts.

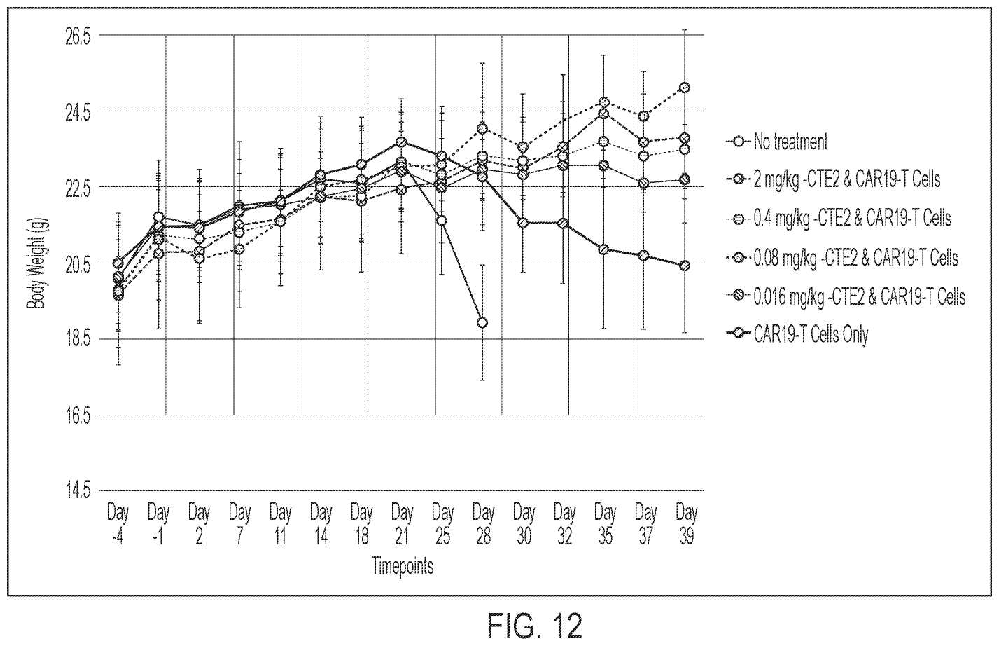

Body weight is a quantitative means of tracking animal health and is typically used in conjunction with clinical examinations to ensure humane euthanasia in cancer models. Mice treated with CAR-19 plus CTE2 protein continued to gain body weight throughout the protein dosing period (until day 31), while the control animals began losing weight at approximately day 25, as shown in FIG. 12. Even after dosing with CTE2 protein stopped, the animals in the treated cohorts maintained body weight through the end of the study (day 42) and the highest dose-treated cohort, that had received 2 mg/kg of CTE2 up until day 31, continued to gain weight with the last recorded weight taken on day 39.

These results show that animals in the three highest dose cohorts were healthy enough to gain weight even after cessation of CTE2 dosing on day 31, whereas the negative control groups (CAR-19 only and ‘no treatment’) steadily lost weight after day 21.

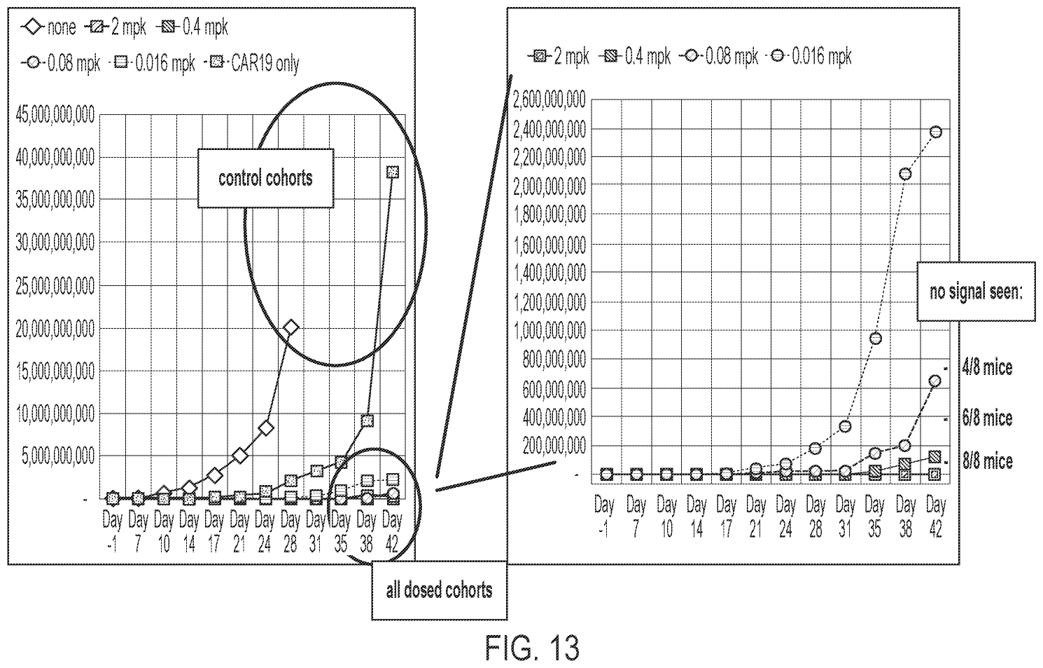

FIG. 13 shows lumen intensity for each cohort, showing a rapid increase in the untreated group and a slightly delayed but still rapid increase in the CAR-19 treated group. The termination of the lines for the untreated and CAR-19-treated groups corresponds to the loss of all animals in those cohorts. Because the signal strength in the control cohorts was so high, it was not possible to discern differences between CTE2 dosed cohorts unless those groups were removed. A clear dose response was evident without those groups, as was a complete lack of luminescent signal in the 2 mg/kg CTE2 cohort through day 42. Since the last protein dose was given on day 31, this suggests that these animals had eliminated the CD19-negative lymphoma.

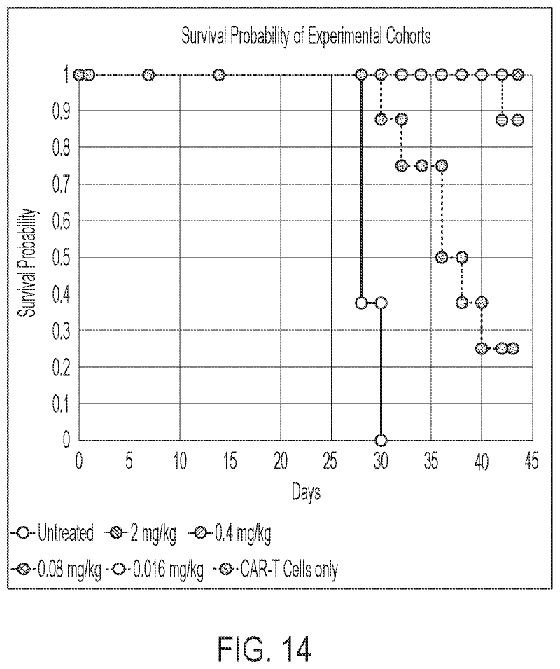

These results show full protection from lymphoma as measured by luminescence in all of the animals treated with 2 mg/kg CTE2 (8/8 animals show no signal), most of the animals treated with 0.4 mg/kg CTE2 ( 6/8 animals show no signal) and half of the animals treated with 0.08 mg/kg CTE2 ( 4/8 animals show no signal). The lack of luminescent signal and the continued weight gain indicated that some mice were free of disease, an indication supported by the Kaplan-Meier survival curves for the different cohorts, as shown in FIG. 14.

The survival data illustrate that only 1 treated animal out of 32 (3%) succumbed to lymphoma during the course of study; that animal was in the lowest dose cohort of 0.016 mg./kg. In contrast, 14/16 (87.5%) of control animals succumbed to lymphoma during the course of study.

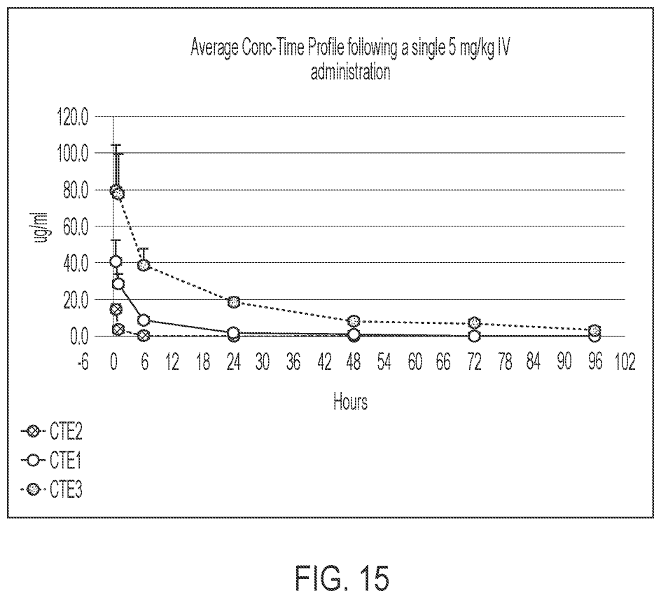

Protein efficacy in vivo is a function of the pharmacokinetic (PK) properties of the injected fusion protein. The PK properties of CTE1, CTE2 and CTE3 were evaluated in mouse serum after a single injection, as shown in FIG. 15. The half-life of the fusion protein in mice depends on albumin-mediated recirculation via FcRN binding since there is no antigen target—the anti-CD20 domain does not bind mouse CD20 and the human CD19 ECD has no measurable binding properties in solution, but the anti-albumin domain can bind to mouse albumin. The CTE1 protein was evaluated in the serum of both Balb/c and NSG mice after a single injection IV and showed a similar half-life of 21 and 28 hours, respectively. The half-life of CTE2 (low sialylation) in Balb/c mice was shorter than expected, at 7.6 hours and CTE3 (high sialylation) had the longest half-life of 36 hours.

Protein half-life can also be impacted by the degree of sialylation, which is the post-translational, covalent, addition of terminal sialic acid to glycosylated proteins. Proteins with more sialyation have longer half-lives in vivo. Table 4 summarizes fusion protein half-life in Balb/c mice, NSG mice, and fusion protein icIEF profiles. The icIEF data showed that while the CTE1 protein contained a mixture of acidic and basic peaks, the CTE2 protein was almost wholly basic, indicating minimal sialylation and CTE3 was almost wholly acid, indicating extensive sialylation. These results explain the differential PK properties of the three protein preparations.

TABLE 4

| | T_{1/2}, | T_{1/2}, | icIEF, % basic | icIEF, % acidic |
|---|---|---|---|---|
| Protein | Balb/c | NSG | pH peaks | pH peaks |
| CTE1 | 20.63 | 27.7 | 26.2 | 73.8 |
| CTE2 | 7.6 | nd | 100 | 0 |
| CTE3 | 36 | nd | 0 | 100 |

### Example 7. Generation and Testing of Additional Fusion Proteins

Additional fusion proteins were assessed for their ability to bind and kill CD19− CD20+ Jeko-1 cells. First, anti-CD20 VHHs (SEQ ID NOs:37-42) that varied in their CDR2 sequences, within SEQ ID 6, were assessed for their ability to bind to CD20 on CD19− CD20+ Jeko-1 cells using FACS. The Table below depicts changes that were made to the CDR2 sequence of SEQ ID NO:37. As shown in the Table 5, the VHHs including the shown CDR2 sequences exhibited various levels of binding, from complete loss of activity to almost no loss of activity, as well as variance in mean fluorescence intensity (MFI).

| SEQ | | |
|---|---|---|
| ID NO: | CDR2 Sequence | Effect on CD20 binding |
| 60 | ITYSGGSP | 100% binding |
| 61 | ISWSGGWI | No binding |
| 62 | ISWSGGSP | Binding, lower MFI |
| 63 | ITYSGGWI | 100% binding |
| 64 | ISWSGGST | Very reduced binding |
| 65 | ITYSGGST | Binding, lower MFI |

Substitution of Thr-Tyr (TY) at the CDR2 N-terminus of SEQ ID NO:37 with Ser-Trp (SW) clearly affected binding. Surprisingly, a highly non-conservative substitution of the Ser-Pro (SP) sequence at the C-terminal end of CDR2 of SEQ ID NO:37 with Trp-Ile (WI) did not affect binding. Additionally, a mutation within CDR3 of the anti-CD20 VHH of SEQ ID NO:37 (AANPTYGSDWNAEN (SEQ ID NO: 66) to AADPTYGSDWNAEN (SEQ ID NO: 67)) had only a modest effect on binding to CD20.

A series of fusion protein constructs was produced, which included a secretion signal peptide, an anti-CD20 VHH, a linker of GGGGSGGGGSGGGGSGGGGS (SEQ ID NO: 68), a portion of a CD19 ECD, a linker of SRGGGGSGGGGSGGGGS (SEQ ID NO: 59), an anti-albumin VHH, and a hexa-histidine tag (SEQ ID NO: 57). The amino acid sequences of the constructs are provided in Table 6:

TABLE 6

| Construct | SEQ |
|---|---|
| # | ID NO: |
| 526 | 6 |
| (CTE1) | |
| 631 | 10 |
| 632 | 14 |
| 633 | 18 |
| 634 | 22 |
| 635 | 26 |
| 636 | 30 |
| 637 | 34 |

The fusion proteins were purified and then assayed for both binding and killing of CD20-positive Jeko-1 CD19KO cells. The binding is summarized in the following Table 7:

TABLE 7

| | 526 | | | | | | | |
|---|---|---|---|---|---|---|---|---|
| | (CTE1) | 631 | 632 | 633 | 634 | 635 | 636 | 637 |
| EC₅₀ | 23.13 | 2263 | 181 | 40.72 | 4401 | 115.9 | 13.94 | 45.05 |
| (ng/ml) | | | | | | | | |
| EC₅₀ | 0.41 | — | 3.21 | 0.72 | — | 2.06 | 0.25 | 0.8 |
| (nM) | | | | | | | | |
| MW | 56.4 | 56.4 | 56.4 | 56.4 | 56.4 | 56.4 | 56.4 | 56.4 |
| (kDa) | | | | | | | | |

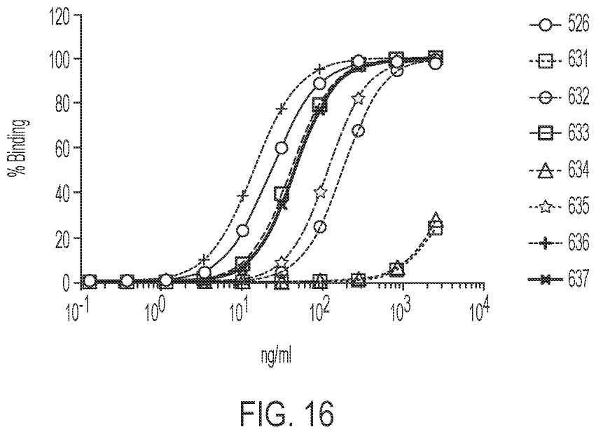

The results of direct cell binding of constructs to CD20 on JeKo-19KO cells, measured by binding of the anti-CD19 mAb FMC63, fully confirmed the preliminary FACS results. Constructs #631 and #634, with four and three AA changes (relative to SEQ ID NO:37), respectively, did not bind the cells at all. Construct #633, with two AA changes relative to SEQ ID NO:37, was almost equipotent with Construct #526 (CTE1). Construct #635 with a conservative change (relative to SEQ ID NO:37) in the C-terminus (Pro to Thr), clearly lost five-fold binding; and Construct #636 (with CDR3 Asn mutated to Asp relative to SEQ ID NO:37), retained full activity. Importantly, combining the mutation in CDR3 with the two AA substitution in Construct #633 resulted in a construct (#637) almost equipotent with Construct #526. The binding curves are illustrated in FIG. 16.

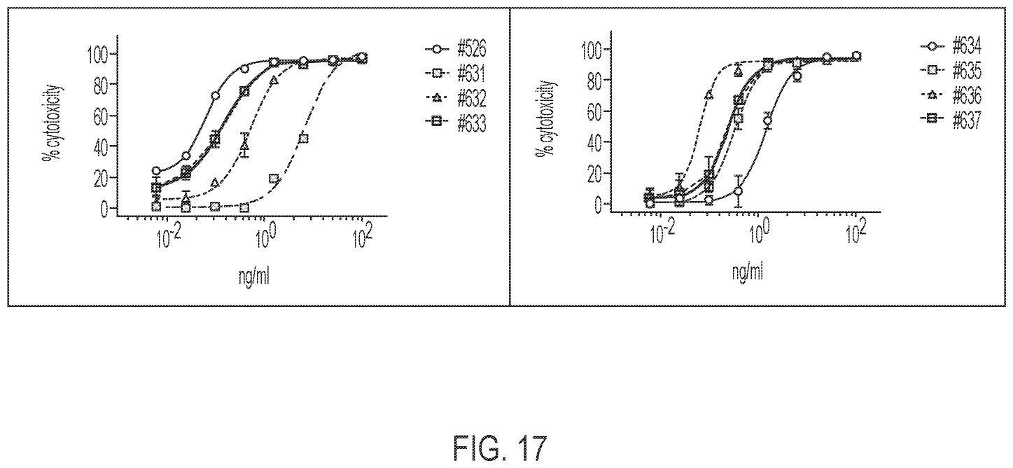

The killing of JeKo-19KO cells by the fusion proteins in the presence of CAR-19s was evaluated. The data are summarized in FIG. 17 and Table 8 below.

TABLE 8

| | EC50 ng/ml | EC50 pM | MW kDa |
|---|---|---|---|
| #526 (CTE1) | 0.063 | 1.13 | 56 |
| #631 | 6.59 | 117.68 | 56 |
| #632 | 0.51 | 9.11 | 56 |
| #633 | 0.14 | 2.5 | 56 |
| #634 | 1.42 | 25.36 | 56 |
| #635 | 0.32 | 5.71 | 56 |
| #636 | 0.063 | 1.13 | 56 |
| #637 | 0.24 | 4.29 | 56 |

FIG. 17 and Table 8 show that the cytotoxicity data are consistent with the binding data.

### Example 8. Assessing Additional Fusion Proteins

An additional fusion protein construct, #607, was produced that contained a different anti-CD20 VHH. The amino acid sequences of the constructs are provided in Table 9:

TABLE 9

| Construct | |
|---|---|
| # | SEQ ID NO: |
| 526 | 6 |
| (CTE1) | |
| 606 | 2, but lacking C-terminal |
| (CTE2) | hexa-histidine tag (SEQ ID NO: |
| | 57) (e.g., SEQ ID NO. 55) |
| 607 | 50 |

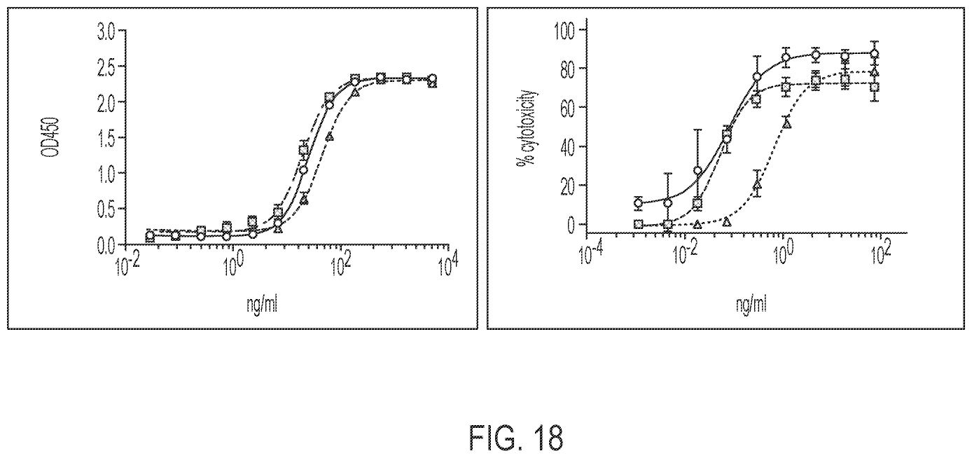

Constructs #526 (CTE1) and #607 bound almost equipotently to cell surface CD20, ˜0.4 nM for Construct #526 (CTE1); ˜0.8 nM for Construct #607 (FIG. 18, left panel). However, Construct #607 was about ten-fold less potent in killing JeKo-19KO cells (FIG. 18, right panel).

TABLE 10:
50
50

| | |
|---|---|
| Binding (EC₅₀) | Cytotoxicity (IC₅₀) |

| construct | MW | ng/ml | nM | ng/ml | pM |
|---|---|---|---|---|---|
| #526 | 56.3 | 25 | 0.44 | 0.078 | 1.39 |
| (CTE1) | | | | | |
| #606 | 56.3 | 19.3 | 0.34 | 0.053 | 0.95 |
| (CTE2) | | | | | |
| #607 | 56.9 | 44.8 | 0.79 | 0.66 | 11.6 |

### Example 9 Biophysical Characterization and Optimization of Biologics

Biologics as described herein are assayed for their biophysical characteristics. A fusion protein as described herein is assessed using standard techniques as known in the art for evaluating characteristics of a biologic for therapy. A fusion protein (e.g., as described in the present Examples) is assessed for stability (e.g., stability in a composition buffer and/or a biological fluid (e.g., blood or saliva).

A fusion protein described in the present Examples is formulated in a preparation that demonstrates optimized melting temperature (Tm), enhanced stability, a reduced level of aggregation and/or degradation, increased level of resistance to proteases, increased level of resistance to oxidation, enhanced biodistribution, and/or improved PK/PD properties in vivo. Such characteristics are assessed using standard methods and assays known in the art, e.g., differential scanning calorimetry (DSC), differential scanning fluorimetry (DSF), circular dichroism (CD), a temperature scanning viscometer, analytical ultracentrifugation (AUC), size exclusion chromatography (SEC, SEC-MALS), dynamic light scattering (DLS), light obscuration, zeta potential, capillary electrophoresis CE (e.g., CZE, MECC, Gel CE, cIEF/iCIEF), gel electrophoresis (e.g., native, SDS-PAGE, IEF), electron microscopy (e.g., TEM, SEM). Formulation parameters that impart these properties include, e.g., pH, osmolality, buffers (e.g., phosphate, acetate, and histidine), tonicity agents/stabilizers (e.g., sugars such as sucrose, trehalose, or mannitol; polyols such as sorbitol), bulking agents (e.g., lyoprotectants such as mannitol), surfactants (e.g., polysorbates), antioxidants (e.g., methionine), metal ions/chelating agents (e.g., ethylenediaminetetraacetic acid, EDTA), and/or preservatives (e.g., benzyl alcohol).

### Example 10. Fusion Proteins Mediation of CAR-19 T Cell Targeting and Cytotoxicity

The present Example further demonstrates that fusion proteins, as described herein, mediate CAR-19 T cell targeting in a concentration-dependent manner. In addition, the present Example further demonstrates that fusion proteins, as described herein, mediate CAR-19 T cell cytotoxicity. These properties, among others, are described in Su et al. Oncoimmunology 2022, Vol. 11, No. 1, e2111904 (13 pages) the entire contents of which are incorporated by reference.

CTE3 fusion protein (described in Example 1), was assessed for CAR-19 T cell targeting and cytotoxicity. Briefly, 96-well round bottom plates were seeded with 50 μL of JeKo-1 CD19KO target cells carrying the luciferase gene at 1×10⁴ cells/well in RPMI 1640 medium containing 10% FBS without antibiotics (RPMI/FBS). Dilutions of test proteins were made in 50 μL RPMI/FBS and added to the cells. The CAR-19 T cells were thawed and washed once with RPMI/FBS, collected via centrifugation at 550 RCF for 10 minutes and added to the wells in a volume of 50 μL to give defined CAR-19 T:target (E:T) cell ratios. The plates were incubated at 37° C. for 48 hours. The plates were centrifuged at 550 RCF for 5 minutes, the pellet was rinsed with PBS, and spun again. Then, 20 μL of 1×lysis buffer (Promega) was added to the pellet, and the lysate was transferred into 96 well opaque tissue culture plates (Fisher Scientific). The plates were read in a luminometer with an injector to dispense the substrate (Promega). The percent killing was calculated based upon the average loss of luminescence of the experimental condition vs the control condition of target cell plus CAR-19 T cell only. In addition, using the “Bang Beads” methodology (Bangs Laboratories, Fishers, IN USA), according to the manufacturer's direction, the number of apparent CD19 molecules on the surface of a JeKo-1 CD19KO target cells, wildtype JeKo-1 Mantle Cell Lymphoma cells or wildtype Ramos Hodgkin Lymphoma cells was determined.

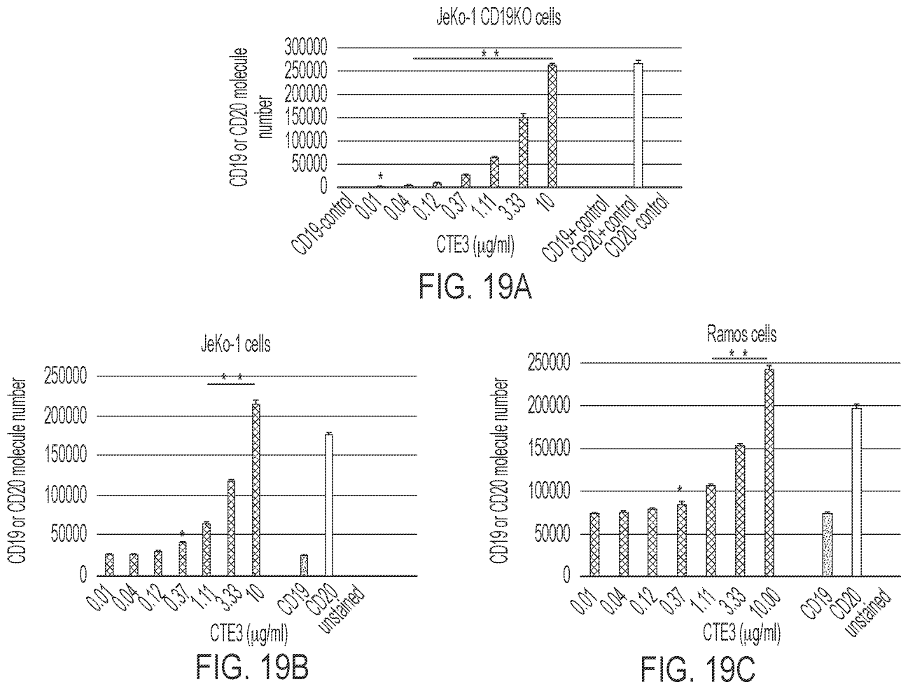

CTE fusion proteins can redirect CAR-19 T cells to CD19 that is bound to and displayed on CD20 cell surface protein. Fusion proteins that bind to CD20 and display the extracellular domain of CD19 can increase the apparent density of CD19 on wildtype lymphoma cells that naturally express both antigens. As shown in FIGS. 19A-19C, the number of apparent CD19 molecules on the surface of a lymphoma cell increased in a concentration-dependent manner when CTE3 fusion protein was added to JeKo-1 CD19KO target cells, wildtype JeKo-1 Mantle Cell Lymphoma cells or wildtype Ramos Hodgkin Lymphoma cells. The total number of CD19 receptors or CD20 receptors on tested cells measured after treatment with 10 μg/mL CTE3 was found to be approximately equal to the number of CD19 receptors plus the number of CD20 receptors.

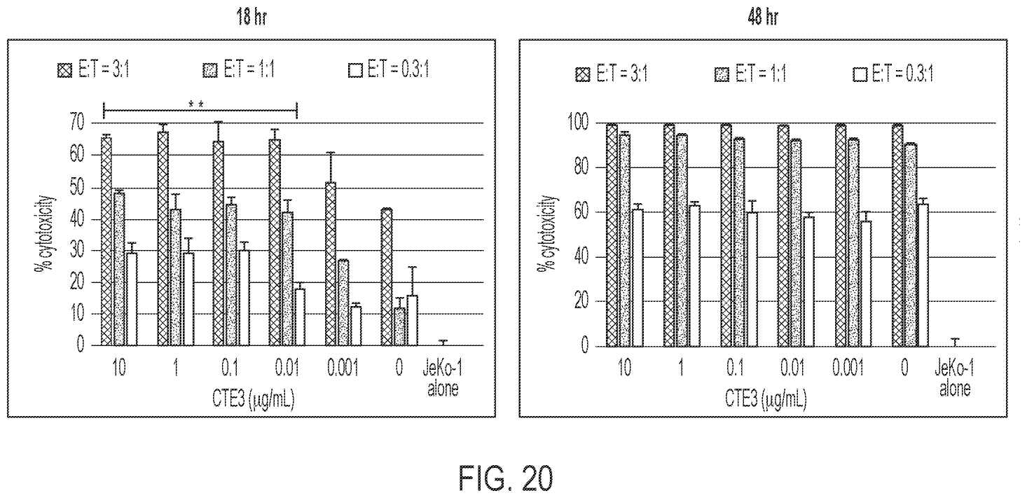

CAR-19 T cells had cytotoxic activity in the presence of target JeKo-19KO cells and CTE proteins. As shown in FIG. 20 (left panel), after 18 hours, CAR-19 T cells had an average of at least 60% cytotoxic activity upon incubation with JeKo-1 cells (E:T 3:1) treated with 0.01, 0.1, 1, or 10 μg/mL CTE proteins. CAR-19 T cells had about an average of at least 50% cytotoxic activity upon incubation with JeKo-1 cells (E:T 3:1) treated with 0.001 μg/mL CTE proteins. After 18 hours, CAR-19 T cells had an average of at least 40% cytotoxic activity upon incubation with JeKo-1 cells (E:T 1:1) treated with 0.01, 0.1, 1, or 10 μg/mL CTE proteins. CAR-19 T cells had about an average of at least 20% cytotoxic activity upon incubation with JeKo-1 cells (E:T 1:1) treated with 0.001 μg/mL CTE proteins. After 18 hours, CAR-19 T cells had an average of about 30% cytotoxic activity upon incubation with JeKo-1 cells (E:T 0.3:1) treated with 0.01, 0.1, 1, or 10 μg/mL CTE proteins. CAR-19 T cells had about an average of at least 10% cytotoxic activity upon incubation with JeKo-1 cells (E:T 0.3:1) treated with 0.001 μg/mL CTE proteins. After 48 hours, CAR-19 T cells exhibited improved cytotoxic activity upon incubation with JeKo-1 cells.

These results further demonstrate that fusion proteins increase the apparent CD19 density on lymphoma cells to a degree that is sufficient to increase CAR-19 mediated cytotoxicity.

### Example 11: CTE3 Binds to Human and Cynomolgus CD20 Expressed on 293T Cells

The present Example further demonstrate that fusion proteins, as described herein, bind to human and cynomolgus CD20.

Human 293T cells were grown to about 80% cell density. The cells were transfected with cDNAs for expression of human CD20 (GenScript OHu00965D) or “cynomolgus” CD20 (an A157V mutation of the human CD20 cDNA) using Lipofectamine 2000 using the manufacturer's instructions (Invitrogen). The A157V mutation is the only amino acid difference from human CD20 in the extracellular domain. The cells were removed by Accutase about 48 hrs post transfection and harvested by centrifugation at 500×g for 2 minutes at 4° C. The cell pellet was resuspended in FACS buffer (PBS with 1% BSA and 0.1% sodium azide). Then, Fc block (BD), at 5 μl per 10⁶ cells, was added to the cell suspension and incubated for 10 minutes at room temperature. Cells were then centrifuged as above and the cells resuspended in FACS buffer and 50 μl (˜5×10⁵ cells) was used per sample. Then, 50 μl of CTE3 fusion protein, starting at 10 μg/ml (final concentration) and then in 3-fold serial dilutions in FACS buffer, was added to the cells. The plate was incubated at 4° C. for 30 minutes, and then centrifuged as above. The cell pellet was washed twice with FACS buffer and then resuspended in 50 μl of diluted FMC63-PE (2.5 μl per 50 μl FACS buffer) and incubated at 4° C. for 30 minutes. Cells were washed twice as above and then fixed with 150 μl of 2% PFA. The samples were analyzed on a BD Accuri C6 flow cytometer using FlowJo software.

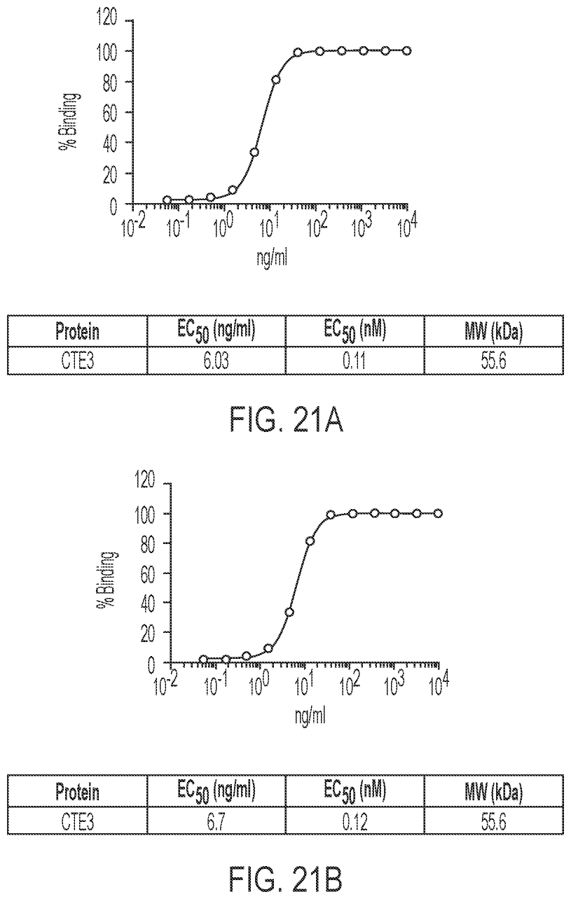

As shown in FIGS. 21A and 21B, the EC50 for CTE3 binding to both human and non-human primate (cynomolgus monkey) CD20 is about 6 ng/ml. These results demonstrate that CTE3 has cross-species specificity between human and non-human primate (cynomolgus monkey).

### Example 12: CTE3 is Active as a CAR T Cell Engager in the Presence of Human Serum or Human Serum Albumin

The present Example further demonstrates that CTE3 remains highly cytotoxic to CD20 expressing cells at pM concentrations.

CTE3 contains an albumin binding domain. An ability to kill CD20 expressing cells in the presence human serum or human serum albumin (HSA) was assessed. The cytotoxicity assay used luciferase-expressing JeKo-1 CD19KO-luc cells to determine the activity of CTE3 in the presence of 50% human serum or HSA at 30 or 60 mg/ml. JeKo-1 CD19KO-luc cells were plated at 1×10⁴ cells in (96 round bottom plate) RPMI medium containing 50% human serum or 10% FBS (control condition) or in 10% FBS RPMI medium containing no, 30 or 60 mg/ml human serum albumin in 50 l/well. CTE3, starting at 100 ng/ml, was titrated with 4-fold serial dilution in the same serum/HSA dilutions as above. The CTE3 dilutions (25 l) were distributed into the corresponding wells. CAR19 T cells at a ratio of 2:1 or 5:1 (CAR T cells to target cells) in RPMI medium containing either 50% human serum or 10% FBS or in 10% FBS RPMI medium containing no, 30 or 60 mg/ml human serum albumin was added in a volume of 25 l/well. The plates were incubated at 37° C. for 48 hours. After washing and pelleting the cells, lysis buffer (Glomax Multi Detection System) was added, the lysates were transferred to a 96 white opaque plate and the luciferase level was measured using a GloMax plate reader (Promega) with an autoinjector. The data was graphed using GraphPad Prism software.

TABLE 11

| CTE3 | IC50 ng/ml | IC50 pM |
|---|---|---|
| In 50% HS | 0.46 | 8.3 |
| In 10% FBS | 0.11 | 2 |

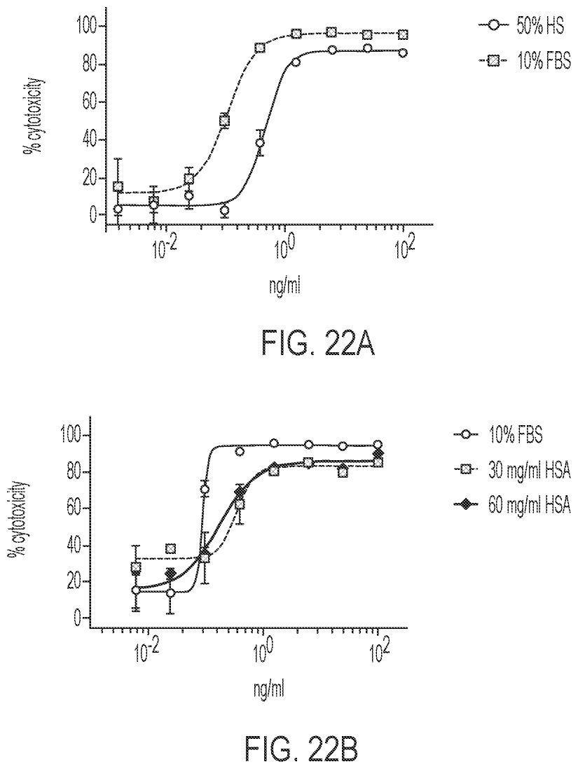

As shown in FIG. 22A, human serum reduces the IC₅₀ of CTE3 about 4-fold as compared to the IC₅₀ for the samples containing 10% FBS. The IC50 values for the cytotoxicity assay were determined and are shown in Table 11. CTE3 is less potent in 50% human serum (IC₅₀ of about 8.3 pM) than in 10% FBS (IC₅₀ of about 2 pM). In the control wells with CAR19 cells, but without CTE3, it was observed that the cell growth in the presence of 50% human serum was reduced as compared to 10% FBS.

TABLE 12

| CTE3 | IC₅₀ (ng/ml) | IC₅₀ pM |
|---|---|---|
| In 10% FBS | 0.09 | 1.62 |
| In 30 mg/ml HSA | 0.35 | 6.3 |
| In 60 mg/ml HSA | 0.18 | 3.2 |

As shown in FIG. 22B, inclusion of HSA, at 30 mg/ml or 60 mg/ml, reduced the ability of CTE3 to induce cytotoxicity of the JeKo-1 CD19 KO-luc cells as compared to the control samples containing 10% FBS. The IC₅₀ values for the cytotoxicity assay were determined and are shown in Table 12. In the presence of HSA, a 2-4 fold decrease in the IC₅₀ was observed and the maximum cytotoxicity level was reduced slightly, compared to the samples assayed in only 10% FBS.

These results demonstrate that although activity is reduced in the presence of 50% human serum or physiological levels of HSA, but CTE3 remains highly cytotoxic to CD20 expressing cells, at pM concentrations.

### Example 13: CD20×CD79b Bispecific Anti-CD19 CAR T Engager Protein Antigen Binding

Bispecific CD79b×CD20 CAR19 engager protein were created by assembling an anti-CD79b scFv with the anti-CD20 VHH and CD19 ECD within CTE3. Constructs were made to place the anti-CD79b scFv at the N-terminus (#650) or the C-terminus (#651) of the the anti-CD20 VHH-CD19 ECD core sequence. The plasmids were transfected into 293T cells using Lipofectamine 2000 and the supernatants harvested and titered. The #650 and #651 proteins were assayed for binding to biotinylated CD20 or CD79b in an ELISA format.

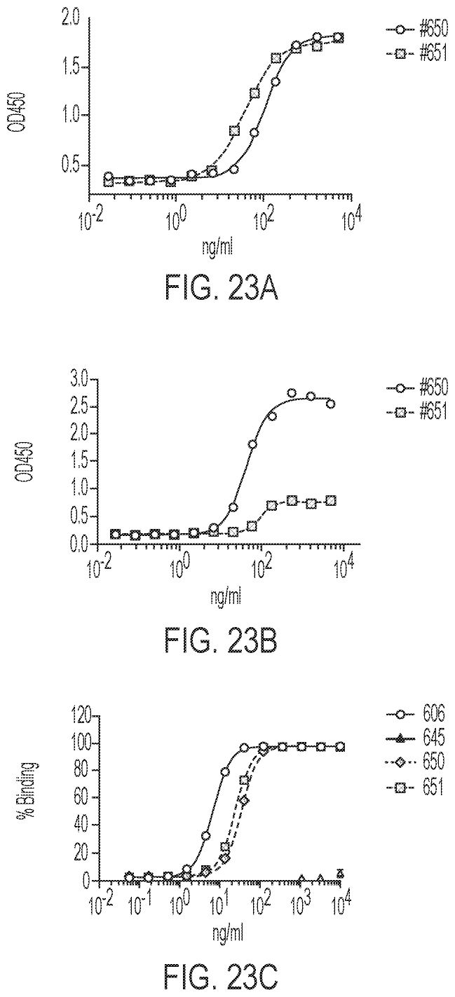

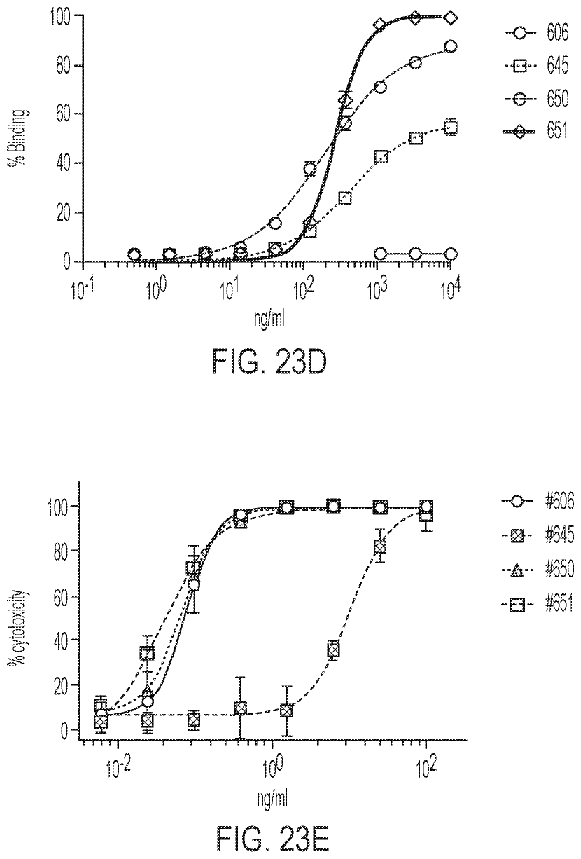

A 96 well plate was coated with 1.0 μg/ml FMC63 in 0.1 M carbonate, pH 9.5 overnight at 4° C. The plate was blocked with 200 μl/well 0.3% non-fat milk in Tris buffered saline (TBS) for 1 hour at room temperature. The plates were then washed three times with wash buffer (1×TBST: 0.1 M Tris, 0.5 M NaCl, 0.05% Tween 20). Next, 100 μl of #650 and #651 supernatant was serially diluted 3-fold, starting at 5 μg/ml, in TBS/1% BSA, pH7.4, (dilution buffer). The samples were incubated for 1 hour at room temperature and then the plates were washed as above. Next, 100 μl of 0.5 μg/ml biotinylated CD20 nanodisc (ACROBiosystems) or biotinylated CD79b (made in house) in dilution buffer was added per well and was incubated for 1 hour at 37° C. After the plates were washed again, 100 μl of a 1:2000 dilution of HRP-Streptavidin (in dilution buffer) was added per well and incubated at 37° C., in the dark for 1 hour. For detection, 100 μl of 1-Step Ultra TMB-ELISA was added per well. When the color developed, 100 μl stop solution was added and the plate was read at 450 nm. Graphs were generated using the Prism software and are shown in FIGS. 23A and 23B.

As shown in FIGS. 23A, placement of CD79b scFv either in the N or C-terminal position to the anti-CD20 VHH does not significantly impact binding to biotinylated CD20. As shown in FIGS. 23B, binding to biotinylated CD79b does appear to be impacted, if the anti-CD79b scFv domain is at the C terminus of the molecule.

The ability to bind cell surface CD20 or CD79b was examined by flow cytometry. The controls for binding to CD20 (#606 anti-CD20 VHH-CD19 ECD-anti-albumin) and to CD79b (#645 anti-CD79b scFv-CD19 ECD) were included in this assay. As shown in FIGS. 23C and 23D, #650 and #651 bind both antigens. Table 13 shows EC₅₀ values for binding to 293T-CD20 cells. Table 14 shows EC₅₀ values for binding to 293T-CD79b cells.

TABLE 13

| | 293T-CD20 binding | | | |
|---|---|---|---|---|
| | 606 | 645 | 650 | 651 |
| EC50 (ng/mL) | 6.70 | — | 34.45 | 24.46 |
| EC50 (nM) | 0.121 | 0.000 | 0.487 | 0.345 |
| MW (Da) | 55560 | 56800 | 70700 | 70800 |

The EC₅₀ for binding CTEs #650 and #651 to cell surface expressed CD20 is similar, but a bit lower than #606.

| | 293T-CD20 binding | | | |
|---|---|---|---|---|
| | 606 | 645 | 650 | 651 |
| EC50 (ng/mL) | — | 425.80 | 200.9 | 269.4 |
| EC50 (nM) | 0.000 | 7.496 | 2.842 | 3.805 |
| MW (Da) | 55560 | 56800 | 70700 | 70800 |

On the CD79b expressing cells, both #650 and #651 bind better than the monospecific anti-CD79b CTE, #645. These results demonstrate that anti-CD20 and anti-CD79b domains can bind their cognate antigen when present on the cell surface.

As shown in FIG. 23E, CD20×CD79b CTEs #650 and #651 were tested for cytotoxic activity against CD20+/CD79b+ JeKo-1 CD19 KO-luc cells. CTE3 was replaced with #606 protein or #645, #650 or #651 supernatants. Table 14 shows IC₅₀ values for cytotoxicity against JeKo-1 CD19 KO cells.

TABLE 14

| | IC₅₀ ng/ml | IC₅₀ pM | MW kDa |
|---|---|---|---|
| #606 | 0.078 | 1.4 | 55.6 |
| #645 | 10.45 | 184.0 | 56.8 |
| #650 | 0.069 | 0.99 | 70.8 |
| #651 | 0.037 | 0.52 | 70.8 |

Monospecific anti-CD20 CTE (#606) and bispecific anti-CD20/anti-CD79b CTEs (#650/#651) can kill JeKo-1 CD19KO cells at IC₅₀ values of ˜1 pM. Anti-CD79b mono specific CTE is less potent.

### Example 14: Surface Plasmon Resonance (SPR) Results for CTE3 Binding to Anti-CD19 FMC63 and HSA

SPR measurements were performed on a Biacore 8K (Cytiva) at 25° C., with 1×HBS-EP+ running buffer. In separate experiments for each ligand, biotinylated anti-CD19 and biotinylated HSA were immobilized onto Streptavidin coated CM5 Biacore Sensor Chips. The biotin/streptavidin interaction is a high affinity, non-covalent interaction, where biotinylated substrates are almost irreversibly bound to the sensor chip surface. This coupling allows all immobilized molecules to be in the same orientation and a high substrate density can be obtained.

The CM5 chip surface was activated by incubation with 1-ethyl-3-(3-dimethylaminopropyl) carbodiimide (EDC)/N-hydroxysuccinimide (NHS). Onto flow cell (FC) 1, free biotin (0.2 pg/ml) was captured: contact time 100 secs at a flow rate of 10 μL/min. Anti-CD19 and HSA were separately diluted into running buffer (1.5 pg/mL and 4.0 pg/mL respectively) and injected over FC2 in separate experiments, until the appropriate level of immobilization was achieved (i.e. 1000-1500 response units (RUs). The remaining streptavidin was deactivated by injection of biotin over all FCs (0.2 pg/ml, contact time 100 secs, at a flow rate of 10 μL/min).

For the binding studies, serial dilutions of CTE3 (3.125, 6.25, 12.5, 25, 50, 100 and 200 nM) were prepared in running buffer and injected over the immobilized target proteins: association time 180 secs, dissociation time 900 secs, flow rate of 30 L/min. Subsequent chip regeneration was performed: regeneration buffer 10 mM Glycine-HCl pH 1.5 for HSA binding, 10 mM Glycine-HCl pH 2.0 for anti-CD19 binding, regeneration time 30 secs twice for HSA binding, 30 secs once for anti-CD19 binding and a flow rate of 10 μL/min. In all experiments, the free biotin immobilized on FC1 served as the reference for blank subtraction.

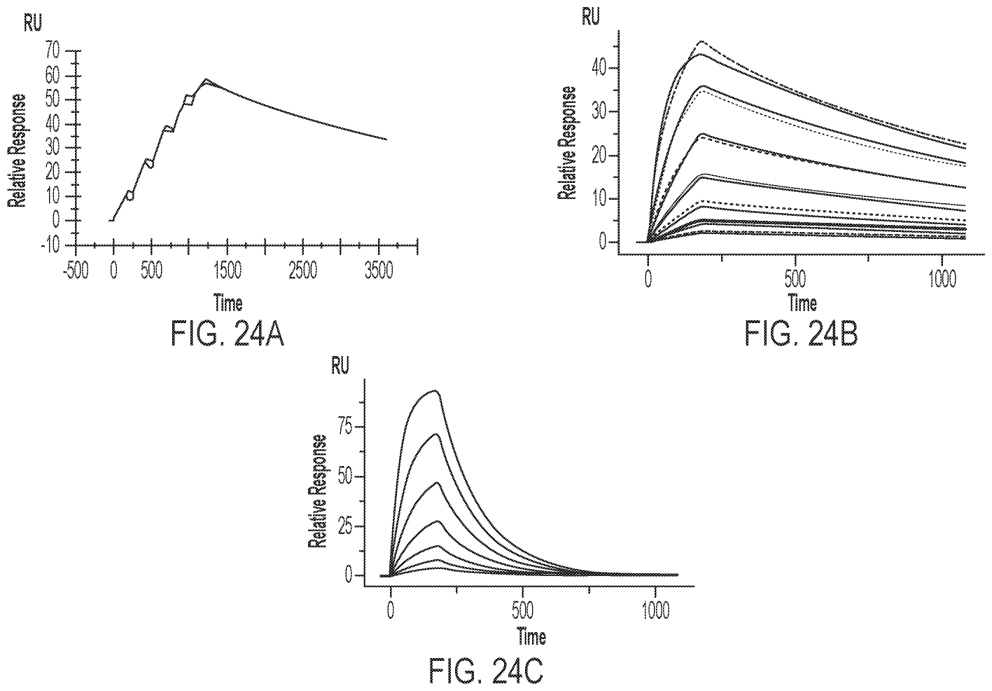

As shown in FIGS. 24B and 24C, association and dissociation curves demonstrated that CTE3 had good affinity for anti-CD19 and HSA, respectively. For anti-CD19, the equilibrium dissociation constant (K_{D}) was 8.13 nM, half life (t½) 15.03 minutes (Table 15). In a similar manner the K_{D} for HSA was determined to be 65.1 nM with a t½ of 1.78 minutes (Table 15).

TABLE 15

| Ligand | Capture Level of Ligand (RU) | kₐ (1/Ms) | k_{d} (1/s) | K_{D} (nM) | t_{1/2} (mins) |
|---|---|---|---|---|---|
| Biotinylated | 156 | 9.92E+04 | 6.46E−03 | 65.1 | 1.78 |
| HSA | | | | | |
| Biotinylated | 472 | 9.45E+04 | 7.68E−04 | 8.13 | 15.03 |
| anti-CD19 | | | | | |

### Example 15: Surface Plasmon Resonance (SPR) Results for CTE3 Binding to CD20

SPR measurements were performed on a Biacore T200 (Cytiva) at 25° C., with running buffer (10 mM HEPES, 150 mM NaCl, 3 mM EDTA, 0.005% P20, 0.05% DDM, 0.01% CHS, pH7.4). Biotinylated CD20 was immobilized onto a Streptavidin coated CM5 Biacore Sensor Chip. The biotin/streptavidin interaction is a high affinity, non-covalent interaction, where biotinylated substrates are almost irreversibly bound to the sensor chip surface. This coupling allows all immobilized molecules to be in the same orientation and a high substrate density can be obtained.

The CM5 chip surface was activated by incubation with 1-ethyl-3-(3-dimethylaminopropyl) carbodiimide (EDC)/N-hydroxysuccinimide (NHS). Onto flow cell (FC) 1, free biotin (0.2 pg/ml) was captured: contact time 100 seconds at a flow rate of 10 μL/min. Biotinylated CD20 was diluted into running buffer (0.2 pg/mL) and injected over FC2, until the appropriate level of immobilization was achieved (i.e. 1000-1500 response units (RUs). The remaining streptavidin was deactivated by injection of biotin over all FCs (0.2 pg/ml, contact time 100 secs at a flow rate of 10 μL/min).

For the binding studies, CTE3 was exchanged into running buffer using a desalting column. Serial dilutions of CTE3 (12.5, 25, 50, 100 and 200 nM) were prepared and injected over the immobilized target proteins: association 180 secs, dissociation 2400 secs, flow rate of 30 μL/min. No regeneration step was performed. In all experiments, the free biotin immobilized on FC1 served as the reference for blank subtraction. All data analysis was performed using the Biacore evaluation software (Cytiva). The resultant association and dissociation curves were fitted with 1:1 binding model fit. Table 16 shows measured kinetic parameters.

As shown in FIG. 24A, association and dissociation curves demonstrated that CTE3 had good affinity for CD20 (K_{D} was 2.93 nM and t_{1/2} 52.23 minutes). Measured kinetic parameters for anti-CD20 are detailed in Table 16.

TABLE 16

| Ligand | Capture Level of Ligand (RU) | kₐ (1/Ms) | k_{d} (1/s) | K_{D} (nM) | t_{1/2} (mins) |
|---|---|---|---|---|---|
| Biotinylated | 200 | 7.56E+04 | 2.21E−04 | 2.93 | 52.23 |
| CD20 | | | | | |

### Example 16: CTE3 Cytotoxicity on CD20 Expressing Cells is Greater than a CD20×CD3 BiTE

The present Example further demonstrates that CTE3 mediate improved CAR-19 T cell cytotoxicity. Specifically, the present example demonstrates that CTE3 is more cytotoxic to CD20 expressing cells than a CD20×CD3 BiTE after repeat stimulation of anti-CD19 CAR T cells

Raji cells (˜10×10⁶ in 1 ml) were treated with Mytomycin C at 0.5 pg/ml at 37° C. for 1 hr to generate B cells for stimulation. The cells were washed twice with RPMI+10% FBS (RIO) and then resuspended at 1×10⁶ cells/ml. The CAR19 cells were stimulated and restimulated every four days with MMC treated Raji by coculturing them at a ratio of 2 CAR19 cells: 1 Raji cell. The cell density was kept at ˜0.5×10⁶ cells/ml. Every four days, right before the restimulation, cells were sampled, and stained with anti-CD3-FITC and anti-Flag-APC to determine the CAR19 expansion; cell number were determined using flow cytometry with Bang Beads. After CAR19 cell concentration was determined, an CTE3 or bispecific anti-CD20×anti-CD3 (BPS Biosciences, cat #: 100836-2) bridged cytotoxicity assay on Jeko-1 CD19 KO cells was set up using the restimulated CAR19 cells. Results were read 48 hrs after assay.

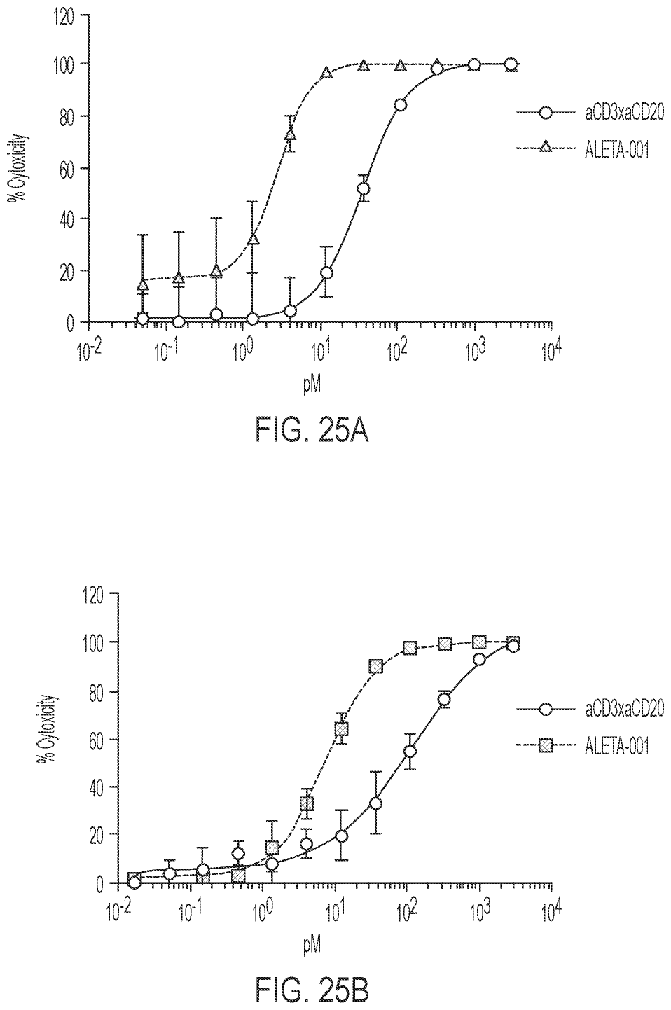

As shown in FIG. 25A, the ability of CTE3 or the anti-CD20×anti-CD3 bispecific antibody to kill Jeko-1 CD19 KO cells with CAR19 cells that have undergone one round of stimulation with Raji cells were assessed. The IC₅₀ for CTE3 is 2.8 pM and the IC₅₀ for the bispecific antibody is 35.7 pM. As shown in FIG. 25B, the potency drops for CAR19 cells having undergone 3 rounds of stimulation. The IC₅₀ for CTE3 remains lower (7.7 pM) than the IC₅₀ for the bispecific (124.1 pM).

These results demonstrate that CTE3 is more cytotoxic than a commercial anti-CD20×anti-CD3 bispecific antibody when CAR19 cells are used in the assay that have been repeated stimulated with B cells. Therefore, CTE3 is likely to be more potent in preventing relapse in CAR19 treated patients.

### Example 17: Batch Analysis for CTE3 Drug Product

The present Example further demonstrates that a composition comprising fusion proteins as described herein may be formulated in a preparation that demonstrates enhanced stability, a reduced level of aggregation and/or degradation, increased level of resistance to proteases, increased level of resistance to oxidation, enhanced biodistribution, and/or improved PK/PD properties in vivo.

Batches of CTE3 were produced and analyzed for various drug product properties. Table 17 shows batch details and release testing for batches of CTE3.

TABLE 17

| | | | Batch Number | |
|---|---|---|---|---|
| | | | 1A | 1B |
| | | | Batch Size/Scale(vials) | |
| | | | 1000 | 3500 |
| | | | Date of Manufacture | |
| | | | 24 Jun. 2022 | 9 Aug. 2022 |
| | | | Use of Batch | |
| | | | Nonclinical, stability | Clinical, stability |
| | | | studies | studies |
| Test | Method | Specification | Result | Result |
| Colour | Ph. Eur. | Not more coloured than | >Y7, <Y6 | >UPW*, <Y7 |
| | <2.2.2> | No. 3 Standard Solution | (Complies) | (Complies) |
| Clarity | Ph. Eur. | ≤18.0 NTU | 3.0 NTU | 2.0 NTU |
| | <2.2.1> | | | |
| pH | Ph. Eur. | 5.5-6.5 | 6.0 | 5.8 |
| | <2.2.3> | | | |
| | USP<791> | | | |
| Osmolality | Ph. Eur. | 250 mOsmol/kg- | 293 mOsmol/kg | 287 mOsmol/kg |
| | <2.2.35> | 350 mOsmol/kg | | |
| | USP<785> | | | |
| Visible | Ph. Eur. | Liquid, essentially free of | Liquid, no visible | Liquid, essentially free of |
| Particles | <2.9.20> | visible particles | particles present | visible particles |
| | USP<790> | | | |
| Subvisible | Ph. Eur. | ≥10 μm: ≤6000 | ≥2 μm: | ≥2 μm: |
| Particulate | <2.9.19> | particles/container | 869 particles/container; | 2198 particles/container; |
| Matter | USP<787> | ≥25 μm: ≤600 | ≥5 μm: | ≥5 μm: |
| | | particles/container | 222 particles/container; | 449 particles/container; |
| | | ≥5 μm: Report result; | ≥10 μm: | ≥10 μm: |
| | | ≥2 μm: Report result. | 26 particles/container; | 62 particles/container; |
| | | | ≥25 μm: | ≥25 μm: |
| | | | 0 particles/container | 2 particles/container |
| Extractable | Ph. Eur. | ≥15.0 mL | >15.0 mL | >15.0 mL |
| Volume | <2.9.17> | | | |
| | USP<1> | | | |
| Protein | UV | 9.0 mg/mL-11.0 mg/mL | 9.9 mg/mL | 10.1 mg/mL |
| Concentration | | | | |
| De-sialylated | De-sialylated | Conforms to Reference | Conforms to Reference | Conforms to Reference |
| iCIEF | iCIEF | Standard | Standard | Standard |
| De-sialylated | De-sialylated | Main Peak %: ≥35.0% | Main Peak %: 55.5%; | Main Peak %:51.5%; |
| iCIEF | iCIEF | Acidic Peaks %: ≤65.0% | Acidic Peaks %: 44.1%; | Acidic Peaks %: 47.8%; |
| | | Basic Peaks %: ≤10.0% | Basic Peaks %: 0.4% | Basic Peaks %: 0.8% |
| iCIEF | iCIEF | Group A: ≥90.0% | Group A: 100.0% | Group A: 100.0% |
| UPLC-SEC | UPLC-SEC | Monomer % (Main | Monomer % (Main | Monomer % (Main |
| | | Peak %): ≥95.0%; | Peak %): 99.3%; | Peak %): 99.3%; |
| | | HMWS %: ≤5.0% | HMWS %: 0.7% | HMWS %: 0.7% |
| De- | De- | Purity %: ≥90.0% | Purity %: 96.7%; | Purity %: 95.7%; |
| glycosylated | glycosylated | LMWS %: ≤10.0% | LMWS %: 3.3% | LMWS %: 4.3% |
| and Reduced- | and | | | |
| CE-SDS | Reduced- | | | |
| | CE-SDS | | | |
| ELISA Binding | ELISA | 50%-150% relative | 104% relative potency | 100% |
| (Anti-CD19 | | potency | | |
| and CD20) | | | | |
| ELISA Binding | ELISA | 50%-150% relative | 116% relative potency | 97% |
| (Anti-CD19 | | potency | | |
| and HSA) | | | | |
| Sterility | Ph. Eur. | No Growth | No Growth | No Growth |
| | <2.6.1> | | | |
| | USP<71> | | | |
| Bacterial | Ph. Eur. | ≤12.50 EU/mL | 2.00 EU/mL | <0.20 EU/mL |
| | <2.6.14> | | | |
| Endotoxins | USP<85> | | | |
| Polysorbate | Polysorbate | 0.010%-0.030% | 0.021% | 0.019% |
| 80 Content | 80 | | | |
| CCIT | USP<1207> | Not Applicable | NA | NA |

Test batch 1B was assessed for stability after 1 month and 3 months. Table 18 shows real-time stability data for test batch 1B.

TABLE 18

| | | T = 1 | T = 3 |
|---|---|---|---|
| Acceptance Criteria | T = 0 | Month | Month |
| Not more colored than | >UPW, <Y7 | >UPW, <Y7 | >UPW, <Y7 |
| No. 3 Standard | (Complies) | (Complies) | (Complies) |
| Solution | | | |
| ≤18.0 NTU | 2.0 NTU | 1.8 NTU | 1.9 NTU |
| 5.5-6.5 | 5.8 | 6.0 | 6.0 |
| Liquid, essentially free | Liquid, essentially | Liquid, essentially | Liquid, essentially |
| of visible particles | free of visible | free of visible | free of visible |
| | particles | particles | particles |
| ≥10 μm: ≤6000 | ≥2 μm: 2198 | ≥2 μm: 1887 | ≥2 μm: 2331 |
| particles/container; | particles/container; | particles/container; | particles/container; |
| ≥25 μm: ≤600 | ≥5 μm: 449 | ≥5 μm: 300 | ≥5 μm: 497 |
| particles/container; | particles/container; | particles/container; | particles/container; |
| ≥5 μm: Report result; | ≥10 μm: 62 | ≥10 μm: 29 | ≥10 μm: 68 |
| ≥2 μm: Report result. | particles/container; | particles/container; | particles/container; |
| | ≥25 μm: 2 | ≥25 μm: 2 | ≥25 μm: 0 |
| | particles/container | particles/container | particles/container |
| 9.0 mg/mL- | 10.1 mg/mL | 9.9 mg/mL | 10.1 mg/mL |
| 11.0 mg/mL | | | |
| Main Peak %: ≥35.0%; | Main Peak %:51.5%; | Main Peak %:53.7%; | Main Peak %:53.8%; |
| Acidic Peaks %: ≤65.0%; | Acidic Peaks %: | Acidic Peaks %: 45.9%; | Acidic Peaks %: 45.7%; |
| Basic Peaks %: ≤10.0% | 47.8%; | Basic Peaks %: 0.4% | Basic Peaks %: 0.4% |
| | Basic Peaks %: | | |
| | 0.8% | | |
| Group A %: ≥90.0% | Group A: 100.0% | NT | NT |
| Monomer % (Main | Monomer % (Main | Monomer % (Main | Monomer % (Main |
| Peak %): ≥95.0%; | Peak %): 99.3%; | Peak %): 99.2%; | Peak %): 99.3%; |
| HMWS %: ≤5.0% | HMWS %: 0.7% | HMWS %: 0.8% | HMWS %: 0.7% |
| Purity %: ≥90.0%; | Purity %: 95.7%; | Purity %: 96.8%; | Purity %: 96.4%; |
| LMWS %: ≤10.0% | LMWS %: 4.3% | LMWS %: 3.2% | LMWS %: 3.6% |
| 0.010%-0.030% | 0.019% | NT | NT |
| 50%-150% relative | 100% | 101% | 99% |
| potency | | | |
| 50%-150% relative | 97% | 100% | 102% |
| potency | | | |
| Pass | NT | NT | NT |

As shown in Table 18, after 1 and 3 months compositions comprising CTE3 demonstrated similar properties as when initially tested.

In a further example, compositions comprising CTE3 are assessed for stability at time-points beyond 3 months. Table 19 describes stability storage conditions and testing time points. Composition comprising CTE3 are stable for at least 3 months.

TABLE 19

| Batch No. | Stability Type | Storage Condition | | Time Points |
|---|---|---|---|---|
| 20220601 | Real-time | −20 ± 5° | C. | T0, 1 M, 3 M, 6 M, 9 M, 12 M, 18 M, 24 M, |
| | | | | 30 M, 36 M, 42 M, 48 M, 60 M |
| | Accelerated | 5 ± 3° | C. | T0, 1 M, 3 M, 6 M |
| | Stress | 25 ± 2° C./ | | T0, 1 M, 2 M, 3 M |
| | | 60 ± 5% RH | | |
| 20220802 | Real-time | −20 ± 5° | C. | T0, 1 M, 3 M, 6 M, 9 M, 12 M, 18 M, 24 M, |
| | | | | 36 M, 48 M, 60 M |
| | Accelerated | 5 ± 3° | C. | T0, 1 M, 3 M, 6 M |

| | | |
|---|---|---|
| Stress | 25 ± 2° C./ | T0, 1 M, 2 M, 3 M |
| | 60 ± 5% RH | |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

LISTING OF SEQUENCES
SEQ ID NO: 1
ATGGGATGGTCATGTATCATCCTTTTTCTGGTAGCAACTGCAACTGGAGTACATAG
CCAGGTGCAATTACAAGAGTCTGGTGGAGGCCTAGCACAAGCGGGCGGTTCCCT
GAGACTCAGCTGCGCTGCTAGCGGACGTACATTTTCCATGGGCTGGTTTCGTCAA
GCTCCTGGCAAGGAACGCGAGTTTGTGGCTGCTATTACCTACTCTGGAGGCTCCC
CTTACTACGCGAGTTCTGTTCGCGGCCGGTTTACAATCTCCCGGGATAATGCTAAG
AATACAGTTTATCTACAAATGAACTCCCTGAAACCCGAGGATACCGCTGTTTATTA
CTGTGCCGCTAACCCAACCTACGGCTCTGATTGGAACGCTGAGAACTGGGGCCA
GGGCACACAGGTGACTGTTAGTTCCGGTGGCGGTGGCAGCGGTGGCGGAGGATC
TGGAGGCGGTGGAAGTGGAGGTGGAGGTTCTCCCGAGGAGCCCCTGGTTGTGA
AAGTCGAGGAAGGCGACACCGCCGCCCTGTGGTGCCTCAAGGGTACCTCTGATG
GACCCACGCAACAATTGACATGGTCTCGTGAATCCCCTCTGAAGCCTTTCCTCAA
GTACAGCCTGGGAGTCCCCGGCCTTGGAGTACATGTCAGGCCAGACGCGATTAG
TGTTGTCATCCGCAATGTTAGTCAGCAGATGGGCGGCTTTTACTTGTGTCAACCC
GGCCCTCCTTCCGAGAAGGCATGGCAGCCTGGCTGGACCGTGAATGTTGAAGGC
AGCGGCGAGCTGTTCCGATGGAACGTCAGTGACCTTGGCGGCCTGGGCTGCGGA
CTGAAGAACAGATCCAGCGAAGGACCAAGTAGCCCTAGCGGCAAGTTGATGTCT
CCAAAATTATATGTTTGGGCAAAGGATAGGCCCGAGATTTGGGAGGGGGAACCCC
CATGCCTGCCTCCACGAGACAGCCTCAATCAAAGCCTGAGTCGAGATCTGACCG
TTGCTCCTGGTTCAACACTGTGGCTGTCATGCGGCGTACCACCGGATTCTGTTTCT
CGCGGGCCCCTGTCTTGGACCCACGTCCATCCTAAGGGTCCCAAGTCCCTCCTGA
GCCTGGAGCTCAAGGACGACCGGCCAGCACGCGATATGTGGGTTATGGGCACAA
GCCTCATGCTGCCCCGAGCAACCGCGCAAGACGCCGGCAAATGGTACTGCCATA
GGGGTAACCTTACCATGAGTTTTCACTTGGAGATCACAGCTAGGCCTTCTCGAGG
TGGCGGTGGCTCCGGAGGCGGCGGATCAGGAGGTGGCGGCAGTGAAGTCCAAC
TTGTGGAGTCTGGCGGAGGCTTGGTCCAACCTGGCAATTCTCTGCGCTTGTCCTG
CGCTGCTAGCGGGTTCACTTTTTCATCTTTCGGAATGTCTTGGGTTAGACAGGCTC
CAGGCAAGGGACTGGAATGGGTTTCGTCCATTAGTGGCAGCGGGTCTGACACTC
TTTACGCGGACAGTGTCAAGGGCAGGTTCACGATATCAAGAGACAACGCAAAAA
CCACACTCTACCTCCAAATGAACTCTTTGAGACCTGAAGATACAGCAGTGTATTAT
TGCACAATTGGTGGCTCTCTGTCCCGCTCGTCCCAAGGGACACTCGTAACGGTCA
GCTCACATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 2
[MGWSCIILFLVATATGVHS]-
[QVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 3
CAGGTGCAATTACAAGAGTCTGGTGGAGGCCTAGCACAAGCGGGCGGTTCCCTG
AGACTCAGCTGCGCTGCTAGCGGACGTACATTTTCCATGGGCTGGTTTCGTCAAG
CTCCTGGCAAGGAACGCGAGTTTGTGGCTGCTATTACCTACTCTGGAGGCTCCCC
TTACTACGCGAGTTCTGTTCGCGGCCGGTTTACAATCTCCCGGGATAATGCTAAGA
ATACAGTTTATCTACAAATGAACTCCCTGAAACCCGAGGATACCGCTGTTTATTAC
TGTGCCGCTAACCCAACCTACGGCTCTGATTGGAACGCTGAGAACTGGGGCCAG
GGCACACAGGTGACTGTTAGTTCCGGTGGCGGTGGCAGCGGTGGCGGAGGATCT
GGAGGCGGTGGAAGTGGAGGTGGAGGTTCTCCCGAGGAGCCCCTGGTTGTGAA
AGTCGAGGAAGGCGACACCGCCGCCCTGTGGTGCCTCAAGGGTACCTCTGATGG
ACCCACGCAACAATTGACATGGTCTCGTGAATCCCCTCTGAAGCCTTTCCTCAAG
TACAGCCTGGGAGTCCCCGGCCTTGGAGTACATGTCAGGCCAGACGCGATTAGT
GTTGTCATCCGCAATGTTAGTCAGCAGATGGGCGGCTTTTACTTGTGTCAACCCG
GCCCTCCTTCCGAGAAGGCATGGCAGCCTGGCTGGACCGTGAATGTTGAAGGCA
GCGGCGAGCTGTTCCGATGGAACGTCAGTGACCTTGGCGGCCTGGGCTGCGGAC
TGAAGAACAGATCCAGCGAAGGACCAAGTAGCCCTAGCGGCAAGTTGATGTCTC
CAAAATTATATGTTTGGGCAAAGGATAGGCCCGAGATTTGGGAGGGGGAACCCCC
ATGCCTGCCTCCACGAGACAGCCTCAATCAAAGCCTGAGTCGAGATCTGACCGTT
GCTCCTGGTTCAACACTGTGGCTGTCATGCGGCGTACCACCGGATTCTGTTTCTC
GCGGGCCCCTGTCTTGGACCCACGTCCATCCTAAGGGTCCCAAGTCCCTCCTGAG
CCTGGAGCTCAAGGACGACCGGCCAGCACGCGATATGTGGGTTATGGGCACAAG
CCTCATGCTGCCCCGAGCAACCGCGCAAGACGCCGGCAAATGGTACTGCCATAG
GGGTAACCTTACCATGAGTTTTCACTTGGAGATCACAGCTAGGCCTTCTCGAGGT
GGCGGTGGCTCCGGAGGCGGCGGATCAGGAGGTGGCGGCAGTGAAGTCCAACT
TGTGGAGTCTGGCGGAGGCTTGGTCCAACCTGGCAATTCTCTGCGCTTGTCCTGC
GCTGCTAGCGGGTTCACTTTTTCATCTTTCGGAATGTCTTGGGTTAGACAGGCTCC
AGGCAAGGGACTGGAATGGGTTTCGTCCATTAGTGGCAGCGGGTCTGACACTCT
TTACGCGGACAGTGTCAAGGGCAGGTTCACGATATCAAGAGACAACGCAAAAAC
CACACTCTACCTCCAAATGAACTCTTTGAGACCTGAAGATACAGCAGTGTATTATT
GCACAATTGGTGGCTCTCTGTCCCGCTCGTCCCAAGGGACACTCGTAACGGTCAG
CTCA
(In order: [VHH (bold denotes CDR1, CDR2, CDR3)]-[first
linker]-[target polypeptide]-[second linker]-[half-life
extension polypeptide])
SEQ ID NO: 4
IQVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 5
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCACATACTCCGGCGGCT
CTCCCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 6
[MEFGLSWVFLVALFRGVQC]-
[QVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 7
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCACATACTCCGGCGGCTC
TCCCTACTATGCCAGCTCCGTGCGGGGCCGGTTCACCATCAGCCGGGACAACGCC
AAGAATACAGTGTACCTGCAGATGAACTCCCTGAAGCCCGAGGACACCGCCGTG
TACTATTGTGCCGCCAATCCTACATATGGCTCCGATTGGAACGCCGAGAATTGGGG
CCAGGGCACCCAGGTGACAGTGTCTAGCGGAGGAGGTGGGTCTGGAGGTGGAG
GATCTGGTGGAGGTGGGTCTGGAGGAGGTGGATCCCCCGAGGAACCTCTAGTGG
TGAAGGTGGAAGAGGGAGATACCGCTGCCCTGTGGTGCCTCAAGGGGACCTCAG
ATGGCCCCACTCAGCAGCTGACCTGGTCTCGGGAGTCCCCGCTTAAACCCTTCTT
AAAATACAGCCTGGGGGTGCCAGGCCTGGGAGTGCACGTGAGGCCCGACGCCAT
CAGCGTGGTTATCCGGAACGTCTCTCAACAGATGGGGGGCTTCTACCTGTGCCAG
CCGGGGCCCCCCTCTGAGAAGGCCTGGCAGCCTGGCTGGACAGTCAATGTGGAG
GGCAGCGGGGAGCTGTTCCGGTGGAATGTTTCGGACCTAGGTGGCCTGGGCTGT
GGCCTGAAGAACAGGTCCTCAGAGGGCCCCAGCTCCCCTTCCGGGAAGCTCATG
AGCCCCAAGCTGTATGTGTGGGCCAAAGACCGCCCTGAGATCTGGGAGGGAGAG
CCTCCGTGTCTCCCACCGAGGGACAGCCTGAACCAGAGCCTCAGCCGGGACCTC
ACCGTTGCCCCTGGCTCCACACTCTGGCTGTCCTGTGGGGTACCCCCTGACTCTG
TGTCCAGGGGCCCCCTCTCCTGGACCCATGTGCACCCCAAGGGGCCTAAGTCATT
GCTGAGCCTAGAGCTGAAGGACGATCGCCCGGCCAGAGATATGTGGGTAATGGG
CACGAGCCTGATGTTGCCCCGGGCCACAGCTCAAGACGCTGGAAAGTGGTATTG
TCACCGTGGCAACCTGACCATGTCATTCCACCTGGAGATCACTGCTCGGCCATCT
AGAGGAGGCGGAGGAAGTGGAGGAGGAGGGAGCGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 8
IQVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 9
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCAGCTGGTCCGGCGGC
TGGATCTACTATGCCAGCTCCGTGCGGGGCCGGTTCACCATCAGCCGGGACAACG
CCAAGAATACAGTGTACCTGCAGATGAACTCCCTGAAGCCCGAGGACACCGCCG
TGTACTATTGTGCCGCCAATCCTACATATGGCTCCGATTGGAACGCCGAGAATTGG
GGCCAGGGCACCCAGGTGACAGTGTCTAGCGGAGGAGGTGGGTCTGGAGGTGG
AGGATCTGGTGGAGGTGGGTCTGGAGGAGGTGGATCCCCCGAGGAACCTCTAGT
GGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTC
TTAAAATACAGCCTGGGGGTGCCAGGCCTGGGAGTGCACGTGAGGCCCGACGCC
ATCAGCGTGGTTATCCGGAACGTCTCTCAACAGATGGGGGGCTTCTACCTGTGCC
AGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCT
GTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCGGGACC
TCACCGTTGCCCCTGGCTCCACACTCTGGCTGTCCTGTGGGGTACCCCCTGACTC
TGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATG
GGCACGAGCCTGATGTTGCCCCGGGCCACAGCTCAAGACGCTGGAAAGTGGTAT
TGTCACCGTGGCAACCTGACCATGTCATTCCACCTGGAGATCACTGCTCGGCCAT
CTAGAGGAGGCGGAGGAAGTGGAGGAGGAGGGAGCGGGGGGGGGGGAAGTGA
AGTTCAGTTGGTCGAATCTGGAGGGGGACTGGTACAGCCTGGCAACTCTCTGCG
ATTGTCTTGTGCTGCTAGTGGTTTTACATTTTCTTCATTTGGCATGAGTTGGGTCCG
ACAAGCCCCAGGAAAAGGGTTGGAATGGGTGAGCAGCATTTCTGGAAGCGGTTC
TGATACACTCTACGCCGACAGCGTAAAAGGCCGGTTCACTATATCTCGCGATAATG
CAAAAACAACACTTTATCTGCAAATGAATAGTCTGCGACCTGAAGATACAGCAGT
ATATTATTGTACAATAGGAGGTAGTCTCAGTAGGTCATCACAAGGAACACTGGTA
ACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 10
[MEFGLSWVFLVALFRGVQC]-
[QVQLQESGGGLAQAGGSLRLSCAASGRTFSMGWFRQAPGKEREFVAAISWSGGW
IYYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 11
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCAGCTGGTCCGGCGGCT
GGATCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGCGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 12
[QVQLQESGGGLAQAGGSLRLSCAASGRTFSMGWFRQAPGKEREFVAAISWSGGW
IYYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 13
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCAGCTGGTCCGGCGGC
TCTCCCTACTATGCCAGCTCCGTGCGGGGCCGGTTCACCATCAGCCGGGACAACG
CCAAGAATACAGTGTACCTGCAGATGAACTCCCTGAAGCCCGAGGACACCGCCG
TGTACTATTGTGCCGCCAATCCTACATATGGCTCCGATTGGAACGCCGAGAATTGG
GGCCAGGGCACCCAGGTGACAGTGTCTAGCGGAGGAGGTGGGTCTGGAGGTGG
AGGATCTGGTGGAGGTGGGTCTGGAGGAGGTGGATCCCCCGAGGAACCTCTAGT
GGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTC
TTAAAATACAGCCTGGGGGTGCCAGGCCTGGGAGTGCACGTGAGGCCCGACGCC
ATCAGCGTGGTTATCCGGAACGTCTCTCAACAGATGGGGGGCTTCTACCTGTGCC
AGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCT
GTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCGGGACC
TCACCGTTGCCCCTGGCTCCACACTCTGGCTGTCCTGTGGGGTACCCCCTGACTC
TGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATG
GGCACGAGCCTGATGTTGCCCCGGGCCACAGCTCAAGACGCTGGAAAGTGGTAT
TGTCACCGTGGCAACCTGACCATGTCATTCCACCTGGAGATCACTGCTCGGCCAT
CTAGAGGAGGCGGAGGAAGTGGAGGAGGAGGGAGCGGGGGGGGGGGAAGTGA
AGTTCAGTTGGTCGAATCTGGAGGGGGACTGGTACAGCCTGGCAACTCTCTGCG
ATTGTCTTGTGCTGCTAGTGGTTTTACATTTTCTTCATTTGGCATGAGTTGGGTCCG
ACAAGCCCCAGGAAAAGGGTTGGAATGGGTGAGCAGCATTTCTGGAAGCGGTTC
TGATACACTCTACGCCGACAGCGTAAAAGGCCGGTTCACTATATCTCGCGATAATG
CAAAAACAACACTTTATCTGCAAATGAATAGTCTGCGACCTGAAGATACAGCAGT
ATATTATTGTACAATAGGAGGTAGTCTCAGTAGGTCATCACAAGGAACACTGGTA
ACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 14
[MEFGLSWVFLVALFRGVQC]-
[QCQVQLQESGGGLAQAGGSLRLSCAASGRTFSMGWFRQAPGKEREFVAAISWSG
GSPYYASSVRGRFTISRDNAKNTVYLQMNSLKPEDTAVYYCAANPTYGSDWNAEN
WGQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 15
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCAGCTGGTCCGGCGGCT
CTCCCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGCGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 16
IQCQVQLQESGGGLAQAGGSLRLSCAASGRTFSMGWFRQAPGKEREFVAAISWSG
GSPYYASSVRGRFTISRDNAKNTVYLQMNSLKPEDTAVYYCAANPTYGSDWNAEN
WGQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP][SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 17
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCACATACTCCGGCGGCT
GGATCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 18
[MEFGLSWVFLVALFRGVQC]-
[QVQLQESGGGLAQAGGSLRLSCAASGRTFSMGWFRQAPGKEREFVAAITYSGGW
IYYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 19
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCACATACTCCGGCGGCTG
GATCTACTATGCCAGCTCCGTGCGGGGCCGGTTCACCATCAGCCGGGACAACGCC
AAGAATACAGTGTACCTGCAGATGAACTCCCTGAAGCCCGAGGACACCGCCGTG
TACTATTGTGCCGCCAATCCTACATATGGCTCCGATTGGAACGCCGAGAATTGGGG
CCAGGGCACCCAGGTGACAGTGTCTAGCGGAGGAGGTGGGTCTGGAGGTGGAG
GATCTGGTGGAGGTGGGTCTGGAGGAGGTGGATCCCCCGAGGAACCTCTAGTGG
TGAAGGTGGAAGAGGGAGATACCGCTGCCCTGTGGTGCCTCAAGGGGACCTCAG
ATGGCCCCACTCAGCAGCTGACCTGGTCTCGGGAGTCCCCGCTTAAACCCTTCTT
AAAATACAGCCTGGGGGTGCCAGGCCTGGGAGTGCACGTGAGGCCCGACGCCAT
CAGCGTGGTTATCCGGAACGTCTCTCAACAGATGGGGGGCTTCTACCTGTGCCAG
CCGGGGCCCCCCTCTGAGAAGGCCTGGCAGCCTGGCTGGACAGTCAATGTGGAG
GGCAGCGGGGAGCTGTTCCGGTGGAATGTTTCGGACCTAGGTGGCCTGGGCTGT
GGCCTGAAGAACAGGTCCTCAGAGGGCCCCAGCTCCCCTTCCGGGAAGCTCATG
AGCCCCAAGCTGTATGTGTGGGCCAAAGACCGCCCTGAGATCTGGGAGGGAGAG
CCTCCGTGTCTCCCACCGAGGGACAGCCTGAACCAGAGCCTCAGCCGGGACCTC
ACCGTTGCCCCTGGCTCCACACTCTGGCTGTCCTGTGGGGTACCCCCTGACTCTG
TGTCCAGGGGCCCCCTCTCCTGGACCCATGTGCACCCCAAGGGGCCTAAGTCATT
GCTGAGCCTAGAGCTGAAGGACGATCGCCCGGCCAGAGATATGTGGGTAATGGG
CACGAGCCTGATGTTGCCCCGGGCCACAGCTCAAGACGCTGGAAAGTGGTATTG
TCACCGTGGCAACCTGACCATGTCATTCCACCTGGAGATCACTGCTCGGCCATCT
AGAGGAGGCGGAGGAAGTGGAGGAGGAGGGAGCGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 20
IQVQLQESGGGLAQAGGSLRLSCAASGRTFSMGWFRQAPGKEREFVAAITYSGGW
IYYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 21
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCAGCTGGTCCGGCGGC
TCTACCTACTATGCCAGCTCCGTGCGGGGCCGGTTCACCATCAGCCGGGACAACG
CCAAGAATACAGTGTACCTGCAGATGAACTCCCTGAAGCCCGAGGACACCGCCG
TGTACTATTGTGCCGCCAATCCTACATATGGCTCCGATTGGAACGCCGAGAATTGG
GGCCAGGGCACCCAGGTGACAGTGTCTAGCGGAGGAGGTGGGTCTGGAGGTGG
AGGATCTGGTGGAGGTGGGTCTGGAGGAGGTGGATCCCCCGAGGAACCTCTAGT
GGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTC
TTAAAATACAGCCTGGGGGTGCCAGGCCTGGGAGTGCACGTGAGGCCCGACGCC
ATCAGCGTGGTTATCCGGAACGTCTCTCAACAGATGGGGGGCTTCTACCTGTGCC
AGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCT
GTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCGGGACC
TCACCGTTGCCCCTGGCTCCACACTCTGGCTGTCCTGTGGGGTACCCCCTGACTC
TGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATG
GGCACGAGCCTGATGTTGCCCCGGGCCACAGCTCAAGACGCTGGAAAGTGGTAT
TGTCACCGTGGCAACCTGACCATGTCATTCCACCTGGAGATCACTGCTCGGCCAT
CTAGAGGAGGCGGAGGAAGTGGAGGAGGAGGGAGCGGGGGGGGGGGAAGTGA
AGTTCAGTTGGTCGAATCTGGAGGGGGACTGGTACAGCCTGGCAACTCTCTGCG
ATTGTCTTGTGCTGCTAGTGGTTTTACATTTTCTTCATTTGGCATGAGTTGGGTCCG
ACAAGCCCCAGGAAAAGGGTTGGAATGGGTGAGCAGCATTTCTGGAAGCGGTTC
TGATACACTCTACGCCGACAGCGTAAAAGGCCGGTTCACTATATCTCGCGATAATG
CAAAAACAACACTTTATCTGCAAATGAATAGTCTGCGACCTGAAGATACAGCAGT
ATATTATTGTACAATAGGAGGTAGTCTCAGTAGGTCATCACAAGGAACACTGGTA
ACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 22
[MEFGLSWVFLVALFRGVQC]-
[QVQLQESGGGLAQAGGSLRLSCAASGRTFSMGWFRQAPGKEREFVAAISWSGGS
TYYASSVRGRFTISRDNAKNTVYLQMNSLKPEDTAVYYCAANPTYGSDWNAENW
GQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 23
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCAGCTGGTCCGGCGGCT
CTACCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 24
[QVQLQESGGGLAQAGGSLRLSCAASGRTFSMGWFRQAPGKEREFVAAISWSGGS
TYYASSVRGRFTISRDNAKNTVYLQMNSLKPEDTAVYYCAANPTYGSDWNAENW
GQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 25
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCACATACTCCGGCGGCT
CTACCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCAATCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 26
[MEFGLSWVFLVALFRGVQC]-
[QCQVQLQESGGGLAQAGGSLRLSCAASGRTFSMGWFRQAPGKEREFVAAITYSG
GSTYYASSVRGRFTISRDNAKNTVYLQMNSLKPEDTAVYYCAANPTYGSDWNAEN
WGQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 27
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCACATACTCCGGCGGCTC
TACCTACTATGCCAGCTCCGTGCGGGGCCGGTTCACCATCAGCCGGGACAACGCC
AAGAATACAGTGTACCTGCAGATGAACTCCCTGAAGCCCGAGGACACCGCCGTG
TACTATTGTGCCGCCAATCCTACATATGGCTCCGATTGGAACGCCGAGAATTGGGG
CCAGGGCACCCAGGTGACAGTGTCTAGCGGAGGAGGTGGGTCTGGAGGTGGAG
GATCTGGTGGAGGTGGGTCTGGAGGAGGTGGATCCCCCGAGGAACCTCTAGTGG
TGAAGGTGGAAGAGGGAGATACCGCTGCCCTGTGGTGCCTCAAGGGGACCTCAG
ATGGCCCCACTCAGCAGCTGACCTGGTCTCGGGAGTCCCCGCTTAAACCCTTCTT
AAAATACAGCCTGGGGGTGCCAGGCCTGGGAGTGCACGTGAGGCCCGACGCCAT
CAGCGTGGTTATCCGGAACGTCTCTCAACAGATGGGGGGCTTCTACCTGTGCCAG
CCGGGGCCCCCCTCTGAGAAGGCCTGGCAGCCTGGCTGGACAGTCAATGTGGAG
GGCAGCGGGGAGCTGTTCCGGTGGAATGTTTCGGACCTAGGTGGCCTGGGCTGT
GGCCTGAAGAACAGGTCCTCAGAGGGCCCCAGCTCCCCTTCCGGGAAGCTCATG
AGCCCCAAGCTGTATGTGTGGGCCAAAGACCGCCCTGAGATCTGGGAGGGAGAG
CCTCCGTGTCTCCCACCGAGGGACAGCCTGAACCAGAGCCTCAGCCGGGACCTC
ACCGTTGCCCCTGGCTCCACACTCTGGCTGTCCTGTGGGGTACCCCCTGACTCTG
TGTCCAGGGGCCCCCTCTCCTGGACCCATGTGCACCCCAAGGGGCCTAAGTCATT
GCTGAGCCTAGAGCTGAAGGACGATCGCCCGGCCAGAGATATGTGGGTAATGGG
CACGAGCCTGATGTTGCCCCGGGCCACAGCTCAAGACGCTGGAAAGTGGTATTG
TCACCGTGGCAACCTGACCATGTCATTCCACCTGGAGATCACTGCTCGGCCATCT
AGAGGAGGCGGAGGAAGTGGAGGAGGAGGGAGCGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 28
[QCQVQLQESGGGLAQAGGSLRLSCAASGRTFSMGWFRQAPGKEREFVAAITYSG
GSTYYASSVRGRFTISRDNAKNTVYLQMNSLKPEDTAVYYCAANPTYGSDWNAEN
WGQGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 29
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCACATACTCCGGCGGCT
CTCCCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCGACCCTACATATGGCTCCGATTGGAACGCCGAGAATTGG
GGCCAGGGCACCCAGGTGACAGTGTCTAGCGGAGGAGGTGGGTCTGGAGGTGG
AGGATCTGGTGGAGGTGGGTCTGGAGGAGGTGGATCCCCCGAGGAACCTCTAGT
GGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTC
TTAAAATACAGCCTGGGGGTGCCAGGCCTGGGAGTGCACGTGAGGCCCGACGCC
ATCAGCGTGGTTATCCGGAACGTCTCTCAACAGATGGGGGGCTTCTACCTGTGCC
AGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCT
GTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCGGGACC
TCACCGTTGCCCCTGGCTCCACACTCTGGCTGTCCTGTGGGGTACCCCCTGACTC
TGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATG
GGCACGAGCCTGATGTTGCCCCGGGCCACAGCTCAAGACGCTGGAAAGTGGTAT
TGTCACCGTGGCAACCTGACCATGTCATTCCACCTGGAGATCACTGCTCGGCCAT
CTAGAGGAGGCGGAGGAAGTGGAGGAGGAGGGAGCGGGGGGGGGGGAAGTGA
AGTTCAGTTGGTCGAATCTGGAGGGGGACTGGTACAGCCTGGCAACTCTCTGCG
ATTGTCTTGTGCTGCTAGTGGTTTTACATTTTCTTCATTTGGCATGAGTTGGGTCCG
ACAAGCCCCAGGAAAAGGGTTGGAATGGGTGAGCAGCATTTCTGGAAGCGGTTC
TGATACACTCTACGCCGACAGCGTAAAAGGCCGGTTCACTATATCTCGCGATAATG
CAAAAACAACACTTTATCTGCAAATGAATAGTCTGCGACCTGAAGATACAGCAGT
ATATTATTGTACAATAGGAGGTAGTCTCAGTAGGTCATCACAAGGAACACTGGTA
ACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 30
[MEFGLSWVFLVALFRGVQC]-
[QVQLQESGGGLAQAGGSLRLSCAASGRTFSMGWFRQAPGKEREFVAAITYSGGSP
YYASSVRGRFTISRDNAKNTVYLQMNSLKPEDTAVYYCAADPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 31
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCACATACTCCGGCGGCTC
TCCCTACTATGCCAGCTCCGTGCGGGGCCGGTTCACCATCAGCCGGGACAACGCC
AAGAATACAGTGTACCTGCAGATGAACTCCCTGAAGCCCGAGGACACCGCCGTG
TACTATTGTGCCGCCGACCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-
[second linker]-[half-life extension polypeptide])
SEQ ID NO: 32
[QVQLQESGGGLAQAGGSLRLSCAASGRTFSMGWFRQAPGKEREFVAAITYSGGSP
YYASSVRGRFTISRDNAKNTVYLQMNSLKPEDTAVYYCAADPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 33
ATGGAGTTTGGGCTGAGCTGGGTTTTCCTCGTTGCTCTTTTTAGAGGTGTCCAGT
GTCAGGTGCAGCTGCAGGAGTCTGGAGGAGGCCTGGCACAGGCAGGAGGCAGC
CTGCGGCTGTCCTGCGCCGCCTCTGGCAGGACCTTCAGCATGGGCTGGTTTAGGC
AGGCACCAGGCAAGGAGAGGGAGTTCGTGGCCGCCATCACATACTCCGGCGGCT
GGATCTACTATGCCAGCTCCGTGCGGGGCCGGTTCACCATCAGCCGGGACAACGC
CAAGAATACAGTGTACCTGCAGATGAACTCCCTGAAGCCCGAGGACACCGCCGT
GTACTATTGTGCCGCCGACCCTACATATGGCTCCGATTGGAACGCCGAGAATTGG
GGCCAGGGCACCCAGGTGACAGTGTCTAGCGGAGGAGGTGGGTCTGGAGGTGG
AGGATCTGGTGGAGGTGGGTCTGGAGGAGGTGGATCCCCCGAGGAACCTCTAGT
GGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTC
TTAAAATACAGCCTGGGGGTGCCAGGCCTGGGAGTGCACGTGAGGCCCGACGCC
ATCAGCGTGGTTATCCGGAACGTCTCTCAACAGATGGGGGGCTTCTACCTGTGCC
AGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCT
GTGGCCTGAAGAACAGGTCCTCAGAGGGCCCCAGCTCCCCTTCCGGGAAGCTCA
TGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCGGGACC
TCACCGTTGCCCCTGGCTCCACACTCTGGCTGTCCTGTGGGGTACCCCCTGACTC
TGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATG
GGCACGAGCCTGATGTTGCCCCGGGCCACAGCTCAAGACGCTGGAAAGTGGTAT
TGTCACCGTGGCAACCTGACCATGTCATTCCACCTGGAGATCACTGCTCGGCCAT
CTAGAGGAGGCGGAGGAAGTGGAGGAGGAGGGAGCGGGGGGGGGGGAAGTGA
AGTTCAGTTGGTCGAATCTGGAGGGGGACTGGTACAGCCTGGCAACTCTCTGCG
ATTGTCTTGTGCTGCTAGTGGTTTTACATTTTCTTCATTTGGCATGAGTTGGGTCCG
ACAAGCCCCAGGAAAAGGGTTGGAATGGGTGAGCAGCATTTCTGGAAGCGGTTC
TGATACACTCTACGCCGACAGCGTAAAAGGCCGGTTCACTATATCTCGCGATAATG
CAAAAACAACACTTTATCTGCAAATGAATAGTCTGCGACCTGAAGATACAGCAGT
ATATTATTGTACAATAGGAGGTAGTCTCAGTAGGTCATCACAAGGAACACTGGTA
ACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 34
[MEFGLSWVFLVALFRGVQC]-
[QVQLQESGGGLAQAGGSLRLSCAASGRTFSMGWFRQAPGKEREFVAAITYSGGW
IYYASSVRGRFTISRDNAKNTVYLQMNSLKPEDTAVYYCAADPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 35
CAGGTGCAGCTGCAGGAGTCTGGAGGAGGCCTGGCACAGGCAGGAGGCAGCCT
GCGGCTGTCCTGCGCCGCCTCTGGCAGGACCTTCAGCATGGGCTGGTTTAGGCA
GGCACCAGGCAAGGAGAGGGAGTTCGTGGCCGCCATCACATACTCCGGCGGCTG
GATCTACTATGCCAGCTCCGTGCGGGGCCGGTTCACCATCAGCCGGGACAACGCC
AAGAATACAGTGTACCTGCAGATGAACTCCCTGAAGCCCGAGGACACCGCCGTG
TACTATTGTGCCGCCGACCCTACATATGGCTCCGATTGGAACGCCGAGAATTGGG
GCCAGGGCACCCAGGTGACAGTGTCTAGCGGAGGAGGTGGGTCTGGAGGTGGA
GGATCTGGTGGAGGTGGGTCTGGAGGAGGTGGATCCCCCGAGGAACCTCTAGTG
GTGAAGGTGGAAGAGGGAGATACCGCTGCCCTGTGGTGCCTCAAGGGGACCTCA
GATGGCCCCACTCAGCAGCTGACCTGGTCTCGGGAGTCCCCGCTTAAACCCTTCT
TAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCA
GCCGGGGCCCCCCTCTGAGAAGGCCTGGCAGCCTGGCTGGACAGTCAATGTGGA
GGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCGGGACCT
CACCGTTGCCCCTGGCTCCACACTCTGGCTGTCCTGTGGGGTACCCCCTGACTCT
GTGTCCAGGGGCCCCCTCTCCTGGACCCATGTGCACCCCAAGGGGCCTAAGTCAT
TGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATT
GTCACCGTGGCAACCTGACCATGTCATTCCACCTGGAGATCACTGCTCGGCCATC
TAGAGGAGGCGGAGGAAGTGGAGGAGGAGGGAGGGGGGGGGGGGAAGTGAA
GTTCAGTTGGTCGAATCTGGAGGGGGACTGGTACAGCCTGGCAACTCTCTGCGAT
TGTCTTGTGCTGCTAGTGGTTTTACATTTTCTTCATTTGGCATGAGTTGGGTCCGA
CAAGCCCCAGGAAAAGGGTTGGAATGGGTGAGCAGCATTTCTGGAAGCGGTTCT
GATACACTCTACGCCGACAGCGTAAAAGGCCGGTTCACTATATCTCGCGATAATGC
AAAAACAACACTTTATCTGCAAATGAATAGTCTGCGACCTGAAGATACAGCAGTA
TATTATTGTACAATAGGAGGTAGTCTCAGTAGGTCATCACAAGGAACACTGGTAA
CCGTGAGTTCC
(In order: [VHH (bold denotes CDR1, CDR2, CDR3)]-[first
linker]-[target polypeptide]-[second linker]-[half-life
extension polypeptide])
SEQ ID NO: 36
[QVQLQESGGGLAQAGGSLRLSCAASGRTFSMGWFRQAPGKEREFVAAITYSGGW
IYYASSVRGRFTISRDNAKNTVYLQMNSLKPEDTAVYYCAADPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
(bold denotes CDR1, CDR2, CDR3)
SEQ ID NO: 37
QVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 38
QVQLQESGGGLAQAGGSLRLSCAASGRTFSMGWFRQAPGKEREFVAAISWSGGWI
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 39
QVQLQESGGGLAQAGGSLRLSCAASGRTFSMGWFRQAPGKEREFVAAISWSGGSP
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 40
QVQLQESGGGLAQAGGSLRLSCAASGRTFSMGWFRQAPGKEREFVAAITYSGGWI
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 41
QVQLQESGGGLAQAGGSLRLSCAASGRTFSMGWFRQAPGKEREFVAAISWSGGST
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 42
QVQLQESGGGLAQAGGSLRLSCAASGRTFSMGWFRQAPGKEREFVAAITYSGGST
YYASSVRGRFTISRDNAKNTVYLQMNSLKPEDTAVYYCAANPTYGSDWNAENWG
QGTQVTVSS
(bold denotes CDR1, CDR2, CDR3)
SEQ ID NO: 43
QVQLQESGGGLAQAGGSLRLSCAASGRTFSMGWFRQAPGKEREFVAAITYSGGSP
YYASSVRGRFTISRDNAKNTVYLQMNSLKPEDTAVYYCAADPTYGSDWNAENWG
QGTQVTVSS
(bold denotes CDR1, CDR2, CDR3)
SEQ ID NO: 44
QVQLQESGGGLAQAGGSLRLSCAASGRTFSMGWFRQAPGKEREFVAAITYSGGWI
YYASSVRGRFTISRDNAKNTVYLQMNSLKPEDTAVYYCAADPTYGSDWNAENWG
QGTQVTVSS
anti-albumin VHH
SEQ ID NO: 45
EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS
SEQ ID NO: 46
PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP
SEQ ID NO: 47
MPPPRLLFFLLFLTPMEVRPEEPLVVKVEEGDNAVLQCLKGTSDGPTQQLTWSRESP
LKPFLKLSLGLPGLGIHMRPLAIWLFIFNVSQQMGGFYLCQPGPPSEKAWQPGWTV
NVEGSGELFRWNVSDLGGLGCGLKNRSSEGPSSPSGKLMSPKLYVWAKDRPEIWEG
EPPCLPPRDSLNQSLSQDLTMAPGSTLWLSCGVPPDSVSRGPLSWTHVHPKGPKSLL
SLELKDDRPARDMWVMETGLLLPRATAQDAGKYYCHRGNLTMSFHLEITARPVLW
HWLLRTGGWKVSAVTLAYLIFCLCSLVGILHLQRALVLRRKRKRMTDPTRRFFKVTP
PPGSGPQNQYGNVLSLPTPTSGLGRAQRWAAGLGGTAPSYGNPSSDVQADGALGSR
SPPGVGPEEEEGEGYEEPDSEEDSEFYENDSNLGQDQLSQDGSGYENPEDEPLGPED
EDSFSNAESYENEDEELTQPVARTMDFLSPHGSAWDPSREATSLGSQSYEDMRGILY
AAPQLRSIRGQPGPNHEEDADSYENMDNPDGPDPAWGGGGRMGTWSTR
SEQ ID NO: 48
PEEPLVVKVEEGDNAVLQCLKGTSDGPTQQLTWSRESPLKPFLKLSLGLPGLGIHMR
PLAIWLFIFNVSQQMGGFYLCQPGPPSEKAWQPGWTVNVEGSGELFRWNVSDLGGL
GCGLKNRSSEGPSSPSGKLMSPKLYVWAKDRPEIWEGEPPCLPPRDSLNQSLSQDLT
MAPGSTLWLSCGVPPDSVSRGPLSWTHVHPKGPKSLLSLELKDDRPARDMWVMET
GLLLPRATAQDAGKYYCHRGNLTMSFHLEITARP
SEQ ID NO: 49
ATGGAGTTTGGGCTGAGCTGGGTTTTCCTCGTTGCTCTTTTTAGAGGTGTCCAGT
GTGAGGTGCAGCTGGTGGAGTCCGGAGGAGGCCTGGTGCAGCCAGGAGGCTCT
CTGAGGCTGAGCTGCACCTTCTCCGGCGGCACCTTCAGCAGCTACACAATGGGC
TGGTTCAGGCAGGCACCAGGCAAGGAGAGAGAGTTTGTGGCAGAGGTGAGGTG
GGGAGGAGTGACCACATACTCCAACTCTCTGAAGGACCGCTTCAGCATCTCCGA
GGATTCTGTGAAGAACGCCGTGTATCTGCAGATGAATAGCCTGAAGCCCGAGGA
CACAGCCGTGTACTATTGTGCCGCCGTGCGGCAGATGTACATGACCGTGGTGCCT
GATTATTGGGGCCAGGGCACCCTGGTGACAGTGTCTAGCGGAGGAGGTGGGTCT
GGAGGTGGAGGATCTGGTGGAGGTGGGTCTGGAGGAGGTGGATCCCCCGAGGA
ACCTCTAGTGGTGAAGGTGGAAGAGGGAGATACCGCTGCCCTGTGGTGCCTCAA
GGGGACCTCAGATGGCCCCACTCAGCAGCTGACCTGGTCTCGGGAGTCCCCGCT
TAAACCCTTCTTAAAATACAGCCTGGGGGTGCCAGGCCTGGGAGTGCACGTGAG
GCCCGACGCCATCAGCGTGGTTATCCGGAACGTCTCTCAACAGATGGGGGGCTTC
TACCTGTGCCAGCCGGGGCCCCCCTCTGAGAAGGCCTGGCAGCCTGGCTGGACA
GTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCC
GGGAAGCTCATGAGCCCCAAGCTGTATGTGTGGGCCAAAGACCGCCCTGAGATC
TGGGAGGGAGAGCCTCCGTGTCTCCCACCGAGGGACAGCCTGAACCAGAGCCT
CAGCCGGGACCTCACCGTTGCCCCTGGCTCCACACTCTGGCTGTCCTGTGGGGTA
CCCCCTGACTCTGTGTCCAGGGGCCCCCTCTCCTGGACCCATGTGCACCCCAAGG
GGCCTAAGTCATTGCTGAGCCTAGAGCTGAAGGACGATCGCCCGGCCAGAGATAT
GTGGGTAATGGGCACGAGCCTGATGTTGCCCCGGGCCACAGCTCAAGACGCTGG
AAAGTGGTATTGTCACCGTGGCAACCTGACCATGTCATTCCACCTGGAGATCACT
GCTCGGCCATCTAGAGGAGGCGGAGGAAGTGGAGGAGGAGGGAGGGGGGGG
GGGGAAGTGAAGTTCAGTTGGTCGAATCTGGAGGGGGACTGGTACAGCCTGGCA
ACTCTCTGCGATTGTCTTGTGCTGCTAGTGGTTTTACATTTTCTTCATTTGGCATGA
GTTGGGTCCGACAAGCCCCAGGAAAAGGGTTGGAATGGGTGAGCAGCATTTCTG
GAAGCGGTTCTGATACACTCTACGCCGACAGCGTAAAAGGCCGGTTCACTATATC
TCGCGATAATGCAAAAACAACACTTTATCTGCAAATGAATAGTCTGCGACCTGAA
GATACAGCAGTATATTATTGTACAATAGGAGGTAGTCTCAGTAGGTCATCACAAGG
AACACTGGTAACCGTGAGTTCCCATCATCACCATCACCAT
(In order: [signal sequence]-[VHH (bold denotes CDR1, CDR2,
CDR3)]-[first linker]-[target polypeptide]-[second linker]-
[half-life extension polypeptide]-[hexa-histidine tag])
SEQ ID NO: 50
[MEFGLSWVFLVALFRGVQC]-
[EVQLVESGGGLVQPGGSLRLSCTFSGGTFSSYTMGWFRQAPGKEREFVAEVRWG
GVTTYSNSLKDRFSISEDSVKNAVYLQMNSLKPEDTAVYYCAAVRQMYMTVVPDY
WGQGTLVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVS
S]-[HHHHHH]
SEQ ID NO: 51
GAGGTGCAGCTGGTGGAGTCCGGAGGAGGCCTGGTGCAGCCAGGAGGCTCTCT
GAGGCTGAGCTGCACCTTCTCCGGCGGCACCTTCAGCAGCTACACAATGGGCTG
GTTCAGGCAGGCACCAGGCAAGGAGAGAGAGTTTGTGGCAGAGGTGAGGTGGG
GAGGAGTGACCACATACTCCAACTCTCTGAAGGACCGCTTCAGCATCTCCGAGG
ATTCTGTGAAGAACGCCGTGTATCTGCAGATGAATAGCCTGAAGCCCGAGGACAC
AGCCGTGTACTATTGTGCCGCCGTGCGGCAGATGTACATGACCGTGGTGCCTGAT
TATTGGGGCCAGGGCACCCTGGTGACAGTGTCTAGCGGAGGAGGTGGGTCTGGA
GGTGGAGGATCTGGTGGAGGTGGGTCTGGAGGAGGTGGATCCCCCGAGGAACCT
CTAGTGGTGAAGGTGGAAGAGGGAGATACCGCTGCCCTGTGGTGCCTCAAGGGG
ACCTCAGATGGCCCCACTCAGCAGCTGACCTGGTCTCGGGAGTCCCCGCTTAAA
CCCTTCTTAAAATACAGCCTGGGGGTGCCAGGCCTGGGAGTGCACGTGAGGCCC
GACGCCATCAGCGTGGTTATCCGGAACGTCTCTCAACAGATGGGGGGCTTCTACC
TGTGCCAGCCGGGGCCCCCCTCTGAGAAGGCCTGGCAGCCTGGCTGGACAGTCA
ATGTGGAGGGCAGCGGGGAGCTGTTCCGGTGGAATGTTTCGGACCTAGGTGGCC
TGGGCTGTGGCCTGAAGAACAGGTCCTCAGAGGGCCCCAGCTCCCCTTCCGGGA
AGCTCATGAGCCCCAAGCTGTATGTGTGGGCCAAAGACCGCCCTGAGATCTGGG
AGGGAGAGCCTCCGTGTCTCCCACCGAGGGACAGCCTGAACCAGAGCCTCAGC
CGGGACCTCACCGTTGCCCCTGGCTCCACACTCTGGCTGTCCTGTGGGGTACCCC
CTGACTCTGTGTCCAGGGGCCCCCTCTCCTGGACCCATGTGCACCCCAAGGGGC
CTAAGTCATTGCTGAGCCTAGAGCTGAAGGACGATCGCCCGGCCAGAGATATGTG
GGTAATGGGCACGAGCCTGATGTTGCCCCGGGCCACAGCTCAAGACGCTGGAAA
GTGGTATTGTCACCGTGGCAACCTGACCATGTCATTCCACCTGGAGATCACTGCT
CGGCCATCTAGAGGAGGCGGAGGAAGTGGAGGAGGAGGGAGCGGGGGGGGGG
GAAGTGAAGTTCAGTTGGTCGAATCTGGAGGGGGACTGGTACAGCCTGGCAACT
CTCTGCGATTGTCTTGTGCTGCTAGTGGTTTTACATTTTCTTCATTTGGCATGAGTT
GGGTCCGACAAGCCCCAGGAAAAGGGTTGGAATGGGTGAGCAGCATTTCTGGA
AGCGGTTCTGATACACTCTACGCCGACAGCGTAAAAGGCCGGTTCACTATATCTC
GCGATAATGCAAAAACAACACTTTATCTGCAAATGAATAGTCTGCGACCTGAAGA
TACAGCAGTATATTATTGTACAATAGGAGGTAGTCTCAGTAGGTCATCACAAGGAA
CACTGGTAACCGTGAGTTCC
(In order: [VHH (bold denotes CDR1, CDR2, CDR3)]-[first
linker]-[target polypeptide]-[second linker]-[half-life
extension polypeptide])
SEQ ID NO: 52
[EVQLVESGGGLVQPGGSLRLSCTFSGGTFSSYTMGWFRQAPGKEREFVAEVRWG
GVTTYSNSLKDRFSISEDSVKNAVYLQMNSLKPEDTAVYYCAAVRQMYMTVVPDY
WGQGTLVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
(bold denotes CDR1, CDR2, CDR3)
SEQ ID NO: 53
EVQLVESGGGLVQPGGSLRLSCTFSGGTFSSYTMGWFRQAPGKEREFVAEVRWGG
VTTYSNSLKDRFSISEDSVKNAVYLQMNSLKPEDTAVYYCAAVRQMYMTVVPDY
WGQGTLVTVSS
SEQ ID NO: 54
ATGGGATGGTCATGTATCATCCTTTTTCTGGTAGCAACTGCAACTGGAGTACATAG
CCAGGTGCAATTACAAGAGTCTGGTGGAGGCCTAGCACAAGCGGGCGGTTCCCT
GAGACTCAGCTGCGCTGCTAGCGGACGTACATTTTCCATGGGCTGGTTTCGTCAA
GCTCCTGGCAAGGAACGCGAGTTTGTGGCTGCTATTACCTACTCTGGAGGCTCCC
CTTACTACGCGAGTTCTGTTCGCGGCCGGTTTACAATCTCCCGGGATAATGCTAAG
AATACAGTTTATCTACAAATGAACTCCCTGAAACCCGAGGATACCGCTGTTTATTA
CTGTGCCGCTAACCCAACCTACGGCTCTGATTGGAACGCTGAGAACTGGGGCCA
GGGCACACAGGTGACTGTTAGTTCCGGTGGCGGTGGCAGCGGTGGCGGAGGATC
TGGAGGCGGTGGAAGTGGAGGTGGAGGTTCTCCCGAGGAGCCCCTGGTTGTGA
AAGTCGAGGAAGGCGACACCGCCGCCCTGTGGTGCCTCAAGGGTACCTCTGATG
GACCCACGCAACAATTGACATGGTCTCGTGAATCCCCTCTGAAGCCTTTCCTCAA
GTACAGCCTGGGAGTCCCCGGCCTTGGAGTACATGTCAGGCCAGACGCGATTAG
TGTTGTCATCCGCAATGTTAGTCAGCAGATGGGCGGCTTTTACTTGTGTCAACCC
GGCCCTCCTTCCGAGAAGGCATGGCAGCCTGGCTGGACCGTGAATGTTGAAGGC
AGCGGCGAGCTGTTCCGATGGAACGTCAGTGACCTTGGCGGCCTGGGCTGCGGA
CTGAAGAACAGATCCAGCGAAGGACCAAGTAGCCCTAGCGGCAAGTTGATGTCT
CCAAAATTATATGTTTGGGCAAAGGATAGGCCCGAGATTTGGGAGGGGGAACCCC
CATGCCTGCCTCCACGAGACAGCCTCAATCAAAGCCTGAGTCGAGATCTGACCG
TTGCTCCTGGTTCAACACTGTGGCTGTCATGCGGCGTACCACCGGATTCTGTTTCT
CGCGGGCCCCTGTCTTGGACCCACGTCCATCCTAAGGGTCCCAAGTCCCTCCTGA
GCCTGGAGCTCAAGGACGACCGGCCAGCACGCGATATGTGGGTTATGGGCACAA
GCCTCATGCTGCCCCGAGCAACCGCGCAAGACGCCGGCAAATGGTACTGCCATA
GGGGTAACCTTACCATGAGTTTTCACTTGGAGATCACAGCTAGGCCTTCTCGAGG
TGGCGGTGGCTCCGGAGGCGGCGGATCAGGAGGTGGCGGCAGTGAAGTCCAAC
TTGTGGAGTCTGGCGGAGGCTTGGTCCAACCTGGCAATTCTCTGCGCTTGTCCTG
CGCTGCTAGCGGGTTCACTTTTTCATCTTTCGGAATGTCTTGGGTTAGACAGGCTC
CAGGCAAGGGACTGGAATGGGTTTCGTCCATTAGTGGCAGCGGGTCTGACACTC
TTTACGCGGACAGTGTCAAGGGCAGGTTCACGATATCAAGAGACAACGCAAAAA
CCACACTCTACCTCCAAATGAACTCTTTGAGACCTGAAGATACAGCAGTGTATTAT
TGCACAATTGGTGGCTCTCTGTCCCGCTCGTCCCAAGGGACACTCGTAACGGTCA
GCTCA
(In order: [signal sequence]-[VHH (bold denotes CDR1, CDR2,
CDR3)]-[first linker]-[target polypeptide]-[second linker]-
[half-life extension polypeptide])
SEQ ID NO: 55
[MGWSCIILFLVATATGVHS]-
[QVQLQESGGGLAQAGGSLRLSCAASGRTFSMGWFRQAPGKEREFVAAITYSGGSP
YYASSVRGRFTISRDNAKNTVYLQMNSLKPEDTAVYYCAANPTYGSDWNAENWG
QGTQVTVSS]-[GGGGSGGGGSGGGGSGGGGS]-
[PEEPLVVKVEEGDTAALWCLKGTSDGPTQQLTWSRESPLKPFLKYSLGVPGLGVHV
RPDAISVVIRNVSQQMGGFYLCQPGPPSEKAWQPGWTVNVEGSGELFRWNVSDLG
GLGCGLKNRSSEGPSSPSGKLMSPKLYVWAKDRPEIWEGEPPCLPPRDSLNQSLSRD
LTVAPGSTLWLSCGVPPDSVSRGPLSWTHVHPKGPKSLLSLELKDDRPARDMWVMG
TSLMLPRATAQDAGKWYCHRGNLTMSFHLEITARP]-[SRGGGGSGGGGSGGGGS]-
[EVQLVESGGGLVQPGNSLRLSCAASGFTFSSFGMSWVRQAPGKGLEWVSSISGSGS
DTLYADSVKGRFTISRDNAKTTLYLQMNSLRPEDTAVYYCTIGGSLSRSSQGTLVTVSS]
SEQ ID NO: 56
PEEPLVVKVEEGDNAVLQCLKGTSDGPTQQLTWSRESPLKPFLKLSLGLPGLGIHMR
PLAIWLFIFNVSQQMGGFYLCQPGPPSEKAWQPGWTVNVEGSGELFRWNVSDLGGL
GCGLKNRSSEGPSSPSGKLMSPKLYVWAKDRPEIWEGEPPCLPPRDSLNQSLSQDLT
MAPGSTLWLSCGVPPDSVSRGPLSWTHVHPKGPKSLLSLELKDDRPARDMWVMET
GLLLPRATAQDAGKYYCHRGNLTMSFHLEITARPVLWHWLLRTGGWKSE

1. A fusion protein comprising (i) an antibody, or antigen binding fragment thereof, that binds a tumor antigen; (ii) a target polypeptide; and (iii) a half-life extension polypeptide.

2. The fusion protein of claim 1, wherein the half-life extension polypeptide is any one of a hyaluronan binding motif, PAS polypeptide, proline/alanine random coil polypeptide, and an antibody or antigen binding fragment thereof.

3. (canceled)

4. The fusion protein of claim 2, wherein the half-life extension polypeptide is an anti-albumin antibody or antigen binding fragment thereof.

5. The fusion protein of claim 4, wherein the anti-albumin antibody or antigen binding fragment thereof comprises an anti-albumin VHH.

6. (canceled)

7. The fusion protein of claim 1, wherein the tumor antigen is CD20, and wherein the antibody or antigen binding fragment thereof is an anti-CD20 antibody or antigen binding fragment thereof.

8. The fusion protein of claim 7, wherein the anti-CD20 antibody or antigen binding fragment thereof comprises an anti-CD20 VHH.

9. (canceled)

10. (canceled)

11. The fusion protein of claim 1, wherein the target polypeptide is a tumor antigen.

12. The fusion protein of claim 1, wherein the target polypeptide is a B cell antigen.

13. (canceled)

14. The fusion protein of claim 12, wherein the target polypeptide is CD19 or a fragment or mutant thereof.

15.-20. (canceled)

21. The fusion protein of claim 1, comprising a first linker between the antibody, or antigen binding fragment thereof, and the target polypeptide.

22. The fusion protein of claim 21, comprising a second linker between the target polypeptide and the half-life extension polypeptide.

23.-25. (canceled)

26. The fusion protein of claim 1, wherein the fusion protein comprises an amino acid sequence having at least about 90%, at least about 95%, or about 100% identity to the amino acid sequence of any one of SEQ ID NOs:2, 4, 6, 8, 14, 16, 18, 20, 26, 28, 30, 32, 34, 36, 50, 52 and 55.

27. (canceled)

28. (canceled)

29. The fusion protein of claim 1, wherein the fusion protein comprises an amino acid sequence having at least about 90%, at least about 95%, or about 100% identity to the amino acid sequence of any one of SEQ ID NOs:2, 6, 14, 18, 26, 30, 34, and 50, and lacking the C-terminal hexa-histidine tag (SEQ ID NO: 57).

30.-33. (canceled)

34. A nucleic acid comprising a nucleotide sequence encoding the amino acid sequence of the fusion protein of claim 1.

35. A nucleic acid comprising (i) the nucleotide sequence of any one of SEQ ID NOs:1, 3, 5, 7, 13, 15, 17, 19, 25, 27, 29, 31, 33, 35, 49, 51 and 54, or (ii) the nucleotide sequence of any one of SEQ ID NOs:1, 5, 9, 13, 17, 21, 25, 29, 33, and 49 and lacking the final 18 nucleotides encoding a hexa-histidine tag (SEQ ID NO: 57).

36. A vector comprising the nucleic acid of claim 34.

37. A host cell comprising the nucleic acid of claim 34.

38. A method of producing a fusion protein, comprising culturing the host cell of claim 34 under conditions suitable for expression of the fusion protein.

39. A method of treating a subject having a tumor, comprising administering to the subject an effective amount of the fusion protein of claim 1, thereby treating the subject.

40. The method of claim 39, wherein the tumor expresses the tumor antigen.

41. The method of claim 39, wherein the tumor does not express CD19.

42. The method of claim 39, wherein upon administration, the fusion protein binds to the tumor antigen.

43. The method of claim 39, further comprising administering to the subject an antibody, an antibody drug conjugate, or a cellular therapeutic (e.g., CAR-T cell) that specifically recognizes the target polypeptide.

44. The method of claim 43, wherein upon administration to the subject, the antibody, antibody drug conjugate, or cellular therapeutic (e.g., CAR-T cell) binds to the fusion protein.

45. The method of claim 44, wherein binding of the antibody, the antibody drug conjugate, or the cellular therapeutic (e.g., CAR-T cell) to the fusion protein induces killing of the tumor.

46. An antibody, or antigen-binding fragment thereof, comprising a VHH having the amino acid sequence of any one of SEQ ID NOs: 37, 39, 40, 42-44 and 53, or a fragment thereof.

47.-51. (canceled)


SEQUENCE LISTING (68 sequences)

SEQ ID NO: 1 - DNA, 1662 bp, synthetic construct
  source 1..1662: mol_type = other DNA
atgggatggtcatgtatcatcctttttctggtagcaactgcaactggagtacatagccaggtgcaattacaagagtctggtggaggcctagcacaagcgggcggttccctgagactcagctgcgctgctagcggacgtacattttccatgggctggtttcgtcaagctcctggcaaggaacgcgagtttgtggctgctattacctactctggaggctccccttactacgcgagttctgttcgcggccggtttacaatctcccgggataatgctaagaatacagtttatctacaaatgaactccctgaaacccgaggataccgctgtttattactgtgccgctaacccaacctacggctctgattggaacgctgagaactggggccagggcacacaggtgactgttagttccggtggcggtggcagcggtggcggaggatctggaggcggtggaagtggaggtggaggttctcccgaggagcccctggttgtgaaagtcgaggaaggcgacaccgccgccctgtggtgcctcaagggtacctctgatggacccacgcaacaattgacatggtctcgtgaatcccctctgaagcctttcctcaagtacagcctgggagtccccggccttggagtacatgtcaggccagacgcgattagtgttgtcatccgcaatgttagtcagcagatgggcggcttttacttgtgtcaacccggccctccttccgagaaggcatggcagcctggctggaccgtgaatgttgaaggcagcggcgagctgttccgatggaacgtcagtgaccttggcggcctgggctgcggactgaagaacagatccagcgaaggaccaagtagccctagcggcaagttgatgtctccaaaattatatgtttgggcaaaggataggcccgagatttgggagggggaacccccatgcctgcctccacgagacagcctcaatcaaagcctgagtcgagatctgaccgttgctcctggttcaacactgtggctgtcatgcggcgtaccaccggattctgtttctcgcgggcccctgtcttggacccacgtccatcctaagggtcccaagtccctcctgagcctggagctcaaggacgaccggccagcacgcgatatgtgggttatgggcacaagcctcatgctgccccgagcaaccgcgcaagacgccggcaaatggtactgccataggggtaaccttaccatgagttttcacttggagatcacagctaggccttctcgaggtggcggtggctccggaggcggcggatcaggaggtggcggcagtgaagtccaacttgtggagtctggcggaggcttggtccaacctggcaattctctgcgcttgtcctgcgctgctagcgggttcactttttcatctttcggaatgtcttgggttagacaggctccaggcaagggactggaatgggtttcgtccattagtggcagcgggtctgacactctttacgcggacagtgtcaagggcaggttcacgatatcaagagacaacgcaaaaaccacactctacctccaaatgaactctttgagacctgaagatacagcagtgtattattgcacaattggtggctctctgtcccgctcgtcccaagggacactcgtaacggtcagctcacatcatcaccatcaccat

SEQ ID NO: 2 - protein, 554 aa, synthetic construct
MGWSCIILFLVATATGVHSQVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 3 - DNA, 1587 bp, synthetic construct
  source 1..1587: mol_type = other DNA
caggtgcaattacaagagtctggtggaggcctagcacaagcgggcggttccctgagactcagctgcgctgctagcggacgtacattttccatgggctggtttcgtcaagctcctggcaaggaacgcgagtttgtggctgctattacctactctggaggctccccttactacgcgagttctgttcgcggccggtttacaatctcccgggataatgctaagaatacagtttatctacaaatgaactccctgaaacccgaggataccgctgtttattactgtgccgctaacccaacctacggctctgattggaacgctgagaactggggccagggcacacaggtgactgttagttccggtggcggtggcagcggtggcggaggatctggaggcggtggaagtggaggtggaggttctcccgaggagcccctggttgtgaaagtcgaggaaggcgacaccgccgccctgtggtgcctcaagggtacctctgatggacccacgcaacaattgacatggtctcgtgaatcccctctgaagcctttcctcaagtacagcctgggagtccccggccttggagtacatgtcaggccagacgcgattagtgttgtcatccgcaatgttagtcagcagatgggcggcttttacttgtgtcaacccggccctccttccgagaaggcatggcagcctggctggaccgtgaatgttgaaggcagcggcgagctgttccgatggaacgtcagtgaccttggcggcctgggctgcggactgaagaacagatccagcgaaggaccaagtagccctagcggcaagttgatgtctccaaaattatatgtttgggcaaaggataggcccgagatttgggagggggaacccccatgcctgcctccacgagacagcctcaatcaaagcctgagtcgagatctgaccgttgctcctggttcaacactgtggctgtcatgcggcgtaccaccggattctgtttctcgcgggcccctgtcttggacccacgtccatcctaagggtcccaagtccctcctgagcctggagctcaaggacgaccggccagcacgcgatatgtgggttatgggcacaagcctcatgctgccccgagcaaccgcgcaagacgccggcaaatggtactgccataggggtaaccttaccatgagttttcacttggagatcacagctaggccttctcgaggtggcggtggctccggaggcggcggatcaggaggtggcggcagtgaagtccaacttgtggagtctggcggaggcttggtccaacctggcaattctctgcgcttgtcctgcgctgctagcgggttcactttttcatctttcggaatgtcttgggttagacaggctccaggcaagggactggaatgggtttcgtccattagtggcagcgggtctgacactctttacgcggacagtgtcaagggcaggttcacgatatcaagagacaacgcaaaaaccacactctacctccaaatgaactctttgagacctgaagatacagcagtgtattattgcacaattggtggctctctgtcccgctcgtcccaagggacactcgtaacggtcagctca

SEQ ID NO: 4 - protein, 529 aa, synthetic construct
QVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 5 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctccctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 6 - protein, 554 aa, synthetic construct
MEFGLSWVFLVALFRGVQCQVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 7 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctccctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 8 - protein, 529 aa, synthetic construct; identical to SEQ ID NO: 4

SEQ ID NO: 9 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctggatctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 10 - protein, 554 aa, synthetic construct
MEFGLSWVFLVALFRGVQCQVQLQESGGGLAQAGGSLRLSCAASGRTFSMGWFRQAPGKEREFVAAISWSGGWIYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 11 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctggatctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 12 - protein, 529 aa, synthetic construct
QVQLQESGGGLAQAGGSLRLSCAASGRTFSMGWFRQAPGKEREFVAAISWSGGWIYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 13 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctctccctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 14 - protein, 556 aa, synthetic construct
MEFGLSWVFLVALFRGVQCQCQVQLQESGGGLAQAGGSLRLSCAASGRTFSMGWFRQAPGKEREFVAAISWSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 15 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctctccctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 16 - protein, 531 aa, synthetic construct
QCQVQLQESGGGLAQAGGSLRLSCAASGRTFSMGWFRQAPGKEREFVAAISWSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 17 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctggatctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 18 - protein, 554 aa, synthetic construct
MEFGLSWVFLVALFRGVQCQVQLQESGGGLAQAGGSLRLSCAASGRTFSMGWFRQAPGKEREFVAAITYSGGWIYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 19 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctggatctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 20 - protein, 529 aa, synthetic construct
QVQLQESGGGLAQAGGSLRLSCAASGRTFSMGWFRQAPGKEREFVAAITYSGGWIYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 21 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctctacctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 22 - protein, 554 aa, synthetic construct
MEFGLSWVFLVALFRGVQCQVQLQESGGGLAQAGGSLRLSCAASGRTFSMGWFRQAPGKEREFVAAISWSGGSTYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 23 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcagctggtccggcggctctacctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 24 - protein, 529 aa, synthetic construct
QVQLQESGGGLAQAGGSLRLSCAASGRTFSMGWFRQAPGKEREFVAAISWSGGSTYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 25 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctacctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 26 - protein, 556 aa, synthetic construct
MEFGLSWVFLVALFRGVQCQCQVQLQESGGGLAQAGGSLRLSCAASGRTFSMGWFRQAPGKEREFVAAITYSGGSTYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 27 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctacctactatgccagctccgtgcggggccggttcaccatcagccgggacaacgccaagaatacagtgtacctgcagatgaactccctgaagcccgaggacaccgccgtgtactattgtgccgccaatcctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 28 - protein, 531 aa, synthetic construct
QCQVQLQESGGGLAQAGGSLRLSCAASGRTFSMGWFRQAPGKEREFVAAITYSGGSTYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 29 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctccctactatgccagctccgtgcggggccggttcaccatcagccgggacaacgccaagaatacagtgtacctgcagatgaactccctgaagcccgaggacaccgccgtgtactattgtgccgccgaccctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 30 - protein, 554 aa, synthetic construct
MEFGLSWVFLVALFRGVQCQVQLQESGGGLAQAGGSLRLSCAASGRTFSMGWFRQAPGKEREFVAAITYSGGSPYYASSVRGRFTISRDNAKNTVYLQMNSLKPEDTAVYYCAADPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 31 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctctccctactatgccagctccgtgcggggccggttcaccatcagccgggacaacgccaagaatacagtgtacctgcagatgaactccctgaagcccgaggacaccgccgtgtactattgtgccgccgaccctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 32 - protein, 529 aa, synthetic construct
QVQLQESGGGLAQAGGSLRLSCAASGRTFSMGWFRQAPGKEREFVAAITYSGGSPYYASSVRGRFTISRDNAKNTVYLQMNSLKPEDTAVYYCAADPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 33 - DNA, 1662 bp, synthetic construct
  source 1..1662: mol_type = other DNA
atggagtttgggctgagctgggttttcctcgttgctctttttagaggtgtccagtgtcaggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctggatctactatgccagctccgtgcggggccggttcaccatcagccgggacaacgccaagaatacagtgtacctgcagatgaactccctgaagcccgaggacaccgccgtgtactattgtgccgccgaccctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 34 - protein, 554 aa, synthetic construct
MEFGLSWVFLVALFRGVQCQVQLQESGGGLAQAGGSLRLSCAASGRTFSMGWFRQAPGKEREFVAAITYSGGWIYYASSVRGRFTISRDNAKNTVYLQMNSLKPEDTAVYYCAADPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 35 - DNA, 1587 bp, synthetic construct
  source 1..1587: mol_type = other DNA
caggtgcagctgcaggagtctggaggaggcctggcacaggcaggaggcagcctgcggctgtcctgcgccgcctctggcaggaccttcagcatgggctggtttaggcaggcaccaggcaaggagagggagttcgtggccgccatcacatactccggcggctggatctactatgccagctccgtgcggggccggttcaccatcagccgggacaacgccaagaatacagtgtacctgcagatgaactccctgaagcccgaggacaccgccgtgtactattgtgccgccgaccctacatatggctccgattggaacgccgagaattggggccagggcacccaggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 36 - protein, 529 aa, synthetic construct
QVQLQESGGGLAQAGGSLRLSCAASGRTFSMGWFRQAPGKEREFVAAITYSGGWIYYASSVRGRFTISRDNAKNTVYLQMNSLKPEDTAVYYCAADPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 37 - protein, 118 aa, synthetic construct
QVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSS

SEQ ID NO: 38 - protein, 118 aa, synthetic construct
QVQLQESGGGLAQAGGSLRLSCAASGRTFSMGWFRQAPGKEREFVAAISWSGGWIYYASSVRGRFTISRDNAKNTVYLQMNSLKPEDTAVYYCAANPTYGSDWNAENWGQGTQVTVSS

SEQ ID NO: 39 - protein, 118 aa, synthetic construct
QVQLQESGGGLAQAGGSLRLSCAASGRTFSMGWFRQAPGKEREFVAAISWSGGSPYYASSVRGRFTISRDNAKNTVYLQMNSLKPEDTAVYYCAANPTYGSDWNAENWGQGTQVTVSS

SEQ ID NO: 40 - protein, 118 aa, synthetic construct
QVQLQESGGGLAQAGGSLRLSCAASGRTFSMGWFRQAPGKEREFVAAITYSGGWIYYASSVRGRFTISRDNAKNTVYLQMNSLKPEDTAVYYCAANPTYGSDWNAENWGQGTQVTVSS

SEQ ID NO: 41 - protein, 118 aa, synthetic construct
QVQLQESGGGLAQAGGSLRLSCAASGRTFSMGWFRQAPGKEREFVAAISWSGGSTYYASSVRGRFTISRDNAKNTVYLQMNSLKPEDTAVYYCAANPTYGSDWNAENWGQGTQVTVSS

SEQ ID NO: 42 - protein, 118 aa, synthetic construct
QVQLQESGGGLAQAGGSLRLSCAASGRTFSMGWFRQAPGKEREFVAAITYSGGSTYYASSVRGRFTISRDNAKNTVYLQMNSLKPEDTAVYYCAANPTYGSDWNAENWGQGTQVTVSS

SEQ ID NO: 43 - protein, 118 aa, synthetic construct
QVQLQESGGGLAQAGGSLRLSCAASGRTFSMGWFRQAPGKEREFVAAITYSGGSPYYASSVRGRFTISRDNAKNTVYLQMNSLKPEDTAVYYCAADPTYGSDWNAENWGQGTQVTVSS

SEQ ID NO: 44 - protein, 118 aa, synthetic construct
QVQLQESGGGLAQAGGSLRLSCAASGRTFSMGWFRQAPGKEREFVAAITYSGGWIYYASSVRGRFTISRDNAKNTVYLQMNSLKPEDTAVYYCAADPTYGSDWNAENWGQGTQVTVSS

SEQ ID NO: 45 - protein, 115 aa, synthetic construct [sequence omitted]

SEQ ID NO: 46 - protein, 259 aa, synthetic construct
PEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARP

SEQ ID NO: 47 - protein, 556 aa, synthetic construct [sequence omitted]

SEQ ID NO: 48 - protein, 259 aa, synthetic construct
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARP

SEQ ID NO: 49 - DNA, 1668 bp, synthetic construct
  source 1..1668: mol_type = other DNA
atggagtttgggctgagctgggttttcctcgttgctctttttagaggtgtccagtgtgaggtgcagctggtggagtccggaggaggcctggtgcagccaggaggctctctgaggctgagctgcaccttctccggcggcaccttcagcagctacacaatgggctggttcaggcaggcaccaggcaaggagagagagtttgtggcagaggtgaggtggggaggagtgaccacatactccaactctctgaaggaccgcttcagcatctccgaggattctgtgaagaacgccgtgtatctgcagatgaatagcctgaagcccgaggacacagccgtgtactattgtgccgccgtgcggcagatgtacatgaccgtggtgcctgattattggggccagggcaccctggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcccatcatcaccatcaccat

SEQ ID NO: 50 - protein, 556 aa, synthetic construct
MEFGLSWVFLVALFRGVQCEVQLVESGGGLVQPGGSLRLSCTFSGGTFSSYTMGWFRQAPGKEREFVAEVRWGGVTTYSNSLKDRFSISEDSVKNAVYLQMNSLKPEDTAVYYCAAVRQMYMTVVPDYWGQGTLVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 51 - DNA, 1593 bp, synthetic construct
  source 1..1593: mol_type = other DNA
gaggtgcagctggtggagtccggaggaggcctggtgcagccaggaggctctctgaggctgagctgcaccttctccggcggcaccttcagcagctacacaatgggctggttcaggcaggcaccaggcaaggagagagagtttgtggcagaggtgaggtggggaggagtgaccacatactccaactctctgaaggaccgcttcagcatctccgaggattctgtgaagaacgccgtgtatctgcagatgaatagcctgaagcccgaggacacagccgtgtactattgtgccgccgtgcggcagatgtacatgaccgtggtgcctgattattggggccagggcaccctggtgacagtgtctagcggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagaggaggcggaggaagtggaggaggagggagcgggggggggggaagtgaagttcagttggtcgaatctggagggggactggtacagcctggcaactctctgcgattgtcttgtgctgctagtggttttacattttcttcatttggcatgagttgggtccgacaagccccaggaaaagggttggaatgggtgagcagcatttctggaagcggttctgatacactctacgccgacagcgtaaaaggccggttcactatatctcgcgataatgcaaaaacaacactttatctgcaaatgaatagtctgcgacctgaagatacagcagtatattattgtacaataggaggtagtctcagtaggtcatcacaaggaacactggtaaccgtgagttcc

SEQ ID NO: 52 - protein, 531 aa, synthetic construct
EVQLVESGGGLVQPGGSLRLSCTFSGGTFSSYTMGWFRQAPGKEREFVAEVRWGGVTTYSNSLKDRFSISEDSVKNAVYLQMNSLKPEDTAVYYCAAVRQMYMTVVPDYWGQGTLVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 53 - protein, 120 aa, synthetic construct
EVQLVESGGGLVQPGGSLRLSCTFSGGTFSSYTMGWFRQAPGKEREFVAEVRWGGVTTYSNSLKDRFSISEDSVKNAVYLQMNSLKPEDTAVYYCAAVRQMYMTVVPDYWGQGTLVTVSS

SEQ ID NO: 54 - DNA, 1644 bp, synthetic construct
  source 1..1644: mol_type = other DNA
atgggatggtcatgtatcatcctttttctggtagcaactgcaactggagtacatagccaggtgcaattacaagagtctggtggaggcctagcacaagcgggcggttccctgagactcagctgcgctgctagcggacgtacattttccatgggctggtttcgtcaagctcctggcaaggaacgcgagtttgtggctgctattacctactctggaggctccccttactacgcgagttctgttcgcggccggtttacaatctcccgggataatgctaagaatacagtttatctacaaatgaactccctgaaacccgaggataccgctgtttattactgtgccgctaacccaacctacggctctgattggaacgctgagaactggggccagggcacacaggtgactgttagttccggtggcggtggcagcggtggcggaggatctggaggcggtggaagtggaggtggaggttctcccgaggagcccctggttgtgaaagtcgaggaaggcgacaccgccgccctgtggtgcctcaagggtacctctgatggacccacgcaacaattgacatggtctcgtgaatcccctctgaagcctttcctcaagtacagcctgggagtccccggccttggagtacatgtcaggccagacgcgattagtgttgtcatccgcaatgttagtcagcagatgggcggcttttacttgtgtcaacccggccctccttccgagaaggcatggcagcctggctggaccgtgaatgttgaaggcagcggcgagctgttccgatggaacgtcagtgaccttggcggcctgggctgcggactgaagaacagatccagcgaaggaccaagtagccctagcggcaagttgatgtctccaaaattatatgtttgggcaaaggataggcccgagatttgggagggggaacccccatgcctgcctccacgagacagcctcaatcaaagcctgagtcgagatctgaccgttgctcctggttcaacactgtggctgtcatgcggcgtaccaccggattctgtttctcgcgggcccctgtcttggacccacgtccatcctaagggtcccaagtccctcctgagcctggagctcaaggacgaccggccagcacgcgatatgtgggttatgggcacaagcctcatgctgccccgagcaaccgcgcaagacgccggcaaatggtactgccataggggtaaccttaccatgagttttcacttggagatcacagctaggccttctcgaggtggcggtggctccggaggcggcggatcaggaggtggcggcagtgaagtccaacttgtggagtctggcggaggcttggtccaacctggcaattctctgcgcttgtcctgcgctgctagcgggttcactttttcatctttcggaatgtcttgggttagacaggctccaggcaagggactggaatgggtttcgtccattagtggcagcgggtctgacactctttacgcggacagtgtcaagggcaggttcacgatatcaagagacaacgcaaaaaccacactctacctccaaatgaactctttgagacctgaagatacagcagtgtattattgcacaattggtggctctctgtcccgctcgtcccaagggacactcgtaacggtcagctca

SEQ ID NO: 55 - protein, 548 aa, synthetic construct
MGWSCIILFLVATATGVHSQVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 56 - protein, 274 aa, synthetic construct
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKSE

SEQ ID NO: 57 - protein, 6 aa, synthetic construct
HHHHHH

SEQ ID NO: 58 - protein, 12 aa, synthetic construct
GSGGGGSGGGGS

SEQ ID NO: 59 - protein, 17 aa, synthetic construct
SRGGGGSGGGGSGGGGS

SEQ ID NO: 60 - protein, 8 aa, synthetic construct
ITYSGGSP

SEQ ID NO: 61 - protein, 8 aa, synthetic construct
ISWSGGWI

SEQ ID NO: 62 - protein, 8 aa, synthetic construct
ISWSGGSP

SEQ ID NO: 63 - protein, 8 aa, synthetic construct
ITYSGGWI

SEQ ID NO: 64 - protein, 8 aa, synthetic construct
ISWSGGST

SEQ ID NO: 65 - protein, 8 aa, synthetic construct
ITYSGGST

SEQ ID NO: 66 - protein, 14 aa, synthetic construct
AANPTYGSDWNAEN

SEQ ID NO: 67 - protein, 14 aa, synthetic construct
AADPTYGSDWNAEN

SEQ ID NO: 68 - protein, 20 aa, synthetic construct
GGGGSGGGGSGGGGSGGGGS